SEQUENCE LISTING (309 sequences)

SEQ ID NO: 1 - protein, 5 aa, synthetic construct
FKVMG

SEQ ID NO: 2 - protein, 16 aa, synthetic construct
TITTGGSPDYSDSVKG

SEQ ID NO: 3 - protein, 13 aa, synthetic construct
RLWAHITSSGHDV

SEQ ID NO: 4 - protein, 5 aa, synthetic construct
GSAMG

SEQ ID NO: 5 - protein, 16 aa, synthetic construct
VIIWTGGYTYYADSVK

SEQ ID NO: 6 - protein, 16 aa, synthetic construct
RTPNSSWFPTTLYEYD

SEQ ID NO: 7 - protein, 5 aa, synthetic construct
NYIMG

SEQ ID NO: 8 - protein, 16 aa, synthetic construct
IITANGVATDYRDSVK

SEQ ID NO: 9 - protein, 15 aa, synthetic construct
RTRSGSLFDPGTFDY

SEQ ID NO: 10 - protein, 121 aa, synthetic construct
DVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSS

SEQ ID NO: 11 - protein, 126 aa, synthetic construct
EVQLVESGGGLAQAGGSLRLSCAPSGRAFSGSAMGWFRQVPGKEREFVGVIIWTGGYTYYADSVKGRFTISRDSAKNTVYLQMDSLKPEDTAVYYCAARTPNSSWFPTTLYEYDYWGQGTQVTVSS

SEQ ID NO: 12 - protein, 124 aa, synthetic construct
EVQLVESGGGLVQAGTSLRLSCAASGRTFSNYIMGWFRQAPGKEREFVAIITANGVATDYRDSVKGRFTISRDNARNTGYLEMNSLKPEDTAVYFCALRTRSGSLFDPGTFDYWGQGTQVTVSS

SEQ ID NO: 13 - protein, 121 aa, synthetic construct
EVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSS

SEQ ID NO: 14-100 - skipped (87 entries)

SEQ ID NO: 101 - protein, 5 aa, synthetic construct
SHWMY

SEQ ID NO: 102 - protein, 16 aa, synthetic construct
EINTNGLITHYGDSVK

SEQ ID NO: 103 - protein, 6 aa, synthetic construct
NQHGLN

SEQ ID NO: 104 - protein, 6 aa, synthetic construct
NQKGLN

SEQ ID NO: 105 - protein, 6 aa, synthetic construct
NQMGLN

SEQ ID NO: 106 - protein, 6 aa, synthetic construct
NERGLN

SEQ ID NO: 107 - protein, 5 aa, synthetic construct
VHWMY

SEQ ID NO: 108 - protein, 5 aa, synthetic construct
NHWMC

SEQ ID NO: 109 - protein, 16 aa, synthetic construct
EINTNGLITHYGDSVH

SEQ ID NO: 110 - protein, 16 aa, synthetic construct
EINTNGLITKYGDSVH

SEQ ID NO: 111 - protein, 16 aa, synthetic construct
EINTNGLITSYVDSVK

SEQ ID NO: 112 - protein, 16 aa, synthetic construct
EINTNGLITKYIDSVR

SEQ ID NO: 113 - protein, 16 aa, synthetic construct
EINTNGLITNYVDSVK

SEQ ID NO: 114 - protein, 16 aa, synthetic construct
EINTNGLITKYIDSVG

SEQ ID NO: 115 - protein, 16 aa, synthetic construct
EINTNGLITKYADFVK

SEQ ID NO: 116 - protein, 16 aa, synthetic construct
EINTNGLITKYADSTK

SEQ ID NO: 117 - protein, 16 aa, synthetic construct
EINTNGLITKYGDSVK

SEQ ID NO: 118 - protein, 115 aa, synthetic construct
DVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSS

SEQ ID NO: 119 - protein, 5 aa, synthetic construct
IHWMY

SEQ ID NO: 120 - protein, 16 aa, synthetic construct
EINTNALITKYADSVK

SEQ ID NO: 121 - protein, 6 aa, synthetic construct
TQNGAA

SEQ ID NO: 122 - protein, 6 aa, synthetic construct
SRNGAA

SEQ ID NO: 123 - protein, 6 aa, synthetic construct
ARNGAA

SEQ ID NO: 124 - protein, 6 aa, synthetic construct
TQNGKT

SEQ ID NO: 125 - protein, 115 aa, synthetic construct
QVQLVESGGGLVQPGGSLRLSCTTSGLDFGIHWMYWFRQAPGKELEWVAEINTNALITKYADSVKGRFTISRDNAKNTLFLQMNDLKSEDTAVYYCSNTQNGAAKGQGVQVTVSS

SEQ ID NO: 126-200 - skipped (75 entries)

SEQ ID NO: 201 - protein, 257 aa, synthetic construct
DVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSSGGGGSGGGGSKGGGGSGGGGSEVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSS

SEQ ID NO: 202 - protein, 257 aa, synthetic construct
DVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSSGGGGSGGGGSKGGGGSGGGGSEVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSS

SEQ ID NO: 203 - protein, 126 aa, synthetic construct
DVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSSGGGGSGGGGSK

SEQ ID NO: 204 - protein, 131 aa, synthetic construct
GGGGSGGGGSEVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSS

SEQ ID NO: 205 - protein, 132 aa, synthetic construct
DVQLVESGGGQVQPGGSLSLSCESSVSIWSFKVMGWFRQVPGKQRELVATITTGGSPDYSDSVKGRFTISRDYDKRTLYLQMNSLKPEDTAVYYCAGRLWAHITSSGHDVWGQGTQVTVSSGGGGSGGGGSK

SEQ ID NO: 206 - protein, 125 aa, synthetic construct
GGGGSGGGGSEVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSS

SEQ ID NO: 207-300 - skipped (94 entries)

SEQ ID NO: 301 - protein, 11 aa, synthetic construct
GGGGSKGGGGS

SEQ ID NO: 302 - protein, 21 aa, synthetic construct
GGGGSGGGGSKGGGGSGGGGS

SEQ ID NO: 303 - protein, 5 aa, synthetic construct
GSGGS

SEQ ID NO: 304 - protein, 5 aa, synthetic construct
GGGGS

SEQ ID NO: 305 - protein, 4 aa, synthetic construct
GGGS

SEQ ID NO: 306 - protein, 7 aa, synthetic construct
AAEPKSS

SEQ ID NO: 307 - protein, 17 aa, synthetic construct
AAEPKSSDKTHTCPPCP

SEQ ID NO: 308 - protein, 4 aa, synthetic construct
GGGG

SEQ ID NO: 309 - protein, 14 aa, synthetic construct
GGGGDKTHTCPPCP

(19) United States
(12) Patent Application Publication
(10) Pub. No.: US 2026/0209332 A1
(43) Pub. Date: Jul. 23, 2026

(54) IL-23 AND TNF-ALPHA INDUCTION TREATMENT FOR AUTOIMMUNE AND INFLAMMATORY DISORDERS

(71) Applicant: SORRISO PHARMACEUTICALS, INC., Sandy, UT (US)

(72) Inventors: Brian Talmadge DORSEY, Encinitas, CA (US); Jacqueline Michelle BENSON, Pacifica, CA (US); Carlos A. SATTLER, La Jolla, CA (US)

(21) Appl. No.: 19/509,037

(22) PCT Filed: Jul. 25, 2024
(86) PCT No.: PCT/US2024/039647

§ 371 Date: Feb. 5, 2026

Related U.S. Application Data
(60) Provisional application No. 63/529,083, filed on Jul. 26, 2023.
(60) Provisional application No. 63/638,352, filed on Apr. 24, 2024.

(51) Int. Cl. C07K 16/24; A61K 9/00; A61K 31/135; A61K 31/397; A61K 31/4045; A61K 31/4245; A61K 31/426; A61K 31/4439; A61K 31/519; A61K 31/5377; A61K 38/08; A61K 38/17; A61K 39/00; A61K 45/06; A61P 1/00; A61P 37/04; C07K 16/28

(52) U.S. Cl. CPC C07K 16/241; A61K 9/0019; A61K 9/0053; A61K 31/135; A61K 31/397; A61K 31/4045; A61K 31/4245; A61K 31/426; A61K 31/4439; A61K 31/519; A61K 31/5377; A61K 38/08; A61K 38/1793; A61K 45/06; A61P 1/00; A61P 37/04; C07K 16/244; C07K 16/2875; A61K 2039/505; A61K 2039/542; A61K 2039/544; A61K 2039/545; C07K 2317/31; C07K 2317/565; C07K 2317/569; C07K 2317/76; C07K 2319/50

(57) ABSTRACT

Described herein is a method of treating an autoimmune and/or inflammatory condition in an individual in need thereof, the method comprising (a) systemic administration of a first inhibitor to the individual in need thereof, wherein the first inhibitor comprises an inhibitor of interleukin-23 (IL-23), an inhibitor of tumor necrosis factor alpha (TNF-alpha), or combinations thereof; and (b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (SIP) modulator, an inhibitor of TNF-like ligand 1 A (TL1 A), or combinations thereof, thereby treating the autoimmune and/or inflammatory condition in the individual in need thereof.

### CROSS-REFERENCE TO RELATED APPLICATIONS

This application claims the benefit of U.S. Provisional Application No. 63/529,083 filed Jul. 26, 2023, and U.S. Provisional Application No. 63/638,352 filed Apr. 24, 2024, the contents of which are incorporated by reference in their entirety.

### SEQUENCE LISTING

The instant application contains a Sequence Listing which has been submitted electronically in XML format and is hereby incorporated by reference in its entirety. Said XML copy, created on Jul. 22, 2024, is named 60790-713_601_SL.xml and is 99,562 bytes in size.

### BACKGROUND

Autoimmune and inflammatory diseases afflict at least 1 in 15 in the United States. Autoimmune and inflammatory conditions arise through dysregulation of the immune system leading to sustained activation of pro-inflammatory pathways, such as constitutive expression of cytokines and chemokines by immune cells, that cause tissue damage. Various autoimmune and inflammatory diseases, such as inflammatory bowel disease (IBD), affect the gastrointestinal (GI) tract. Several autoimmune and inflammatory conditions of the GI tract, such as IBD and celiac disease, are presently incurable which leaves afflicted patients with life-long symptoms. Cytokines have been discovered to play a critical role in the pathogenesis of autoimmune diseases and inflammatory conditions of the GI tract. In particular, interleukin (IL)-23 and tumor necrosis factor alpha (TNF-alpha) have been implicated as key drivers of autoimmune and inflammatory pathologies.

### SUMMARY

Clinical research has shown that IL-23 and/or TNF-alpha blockade can be effective in ameliorating autoimmune and inflammatory conditions in the GI tract. Pre-clinical and clinical data also suggest that the treatment regimen of IL-23 and/or TNF-alpha blockade can impact the efficacy and durability of clinical response. Treatment regimen parameters include, but are not limited to, the administration route, the treatment schedule, and the therapeutic dosage. Treatment for autoimmune and inflammatory conditions of the GI tract can comprise at least two treatment regimens—a first induction phase that is followed by a second maintenance phase. The objective of the first induction phase is to induce remission of autoimmune and inflammatory conditions in the GI tract. The objective of the second maintenance phase is to maintain remission of autoimmune and inflammatory conditions through continued administration of the therapeutic. For antibody therapeutics, both the induction phase and maintenance phase are commonly administered systemically (e.g., intravenous or subcutaneous administration). A sequential induction phase followed by a maintenance phase of IL-23 and/or TNF-alpha blockade may potentiate long-term management of autoimmune and inflammatory conditions of the GI tract. Biologics and small molecules targeting the signaling pathways of sphingosine 1-phosphate (S1P), Janus kinases (JAKs) family, TNF-like ligand 1A (TL1A), or combinations thereof, can be also be incorporated into the or the induction phase maintenance phase.

However, inclusion of an induction phase and/or a maintenance phase of systemic IBD treatment may (a) expose patients to prolonged systemic side effects and (b) impose logistical burdens such as continual trips to the clinic (e.g., intravenous administration) or administration-associated discomfort (e.g., subcutaneous self-administration). Thus, an orally delivered IBD treatment modality involving IL-23 and/or TNF-alpha blockade within IBD tissue that (a) reduces excess systemic exposure of the therapeutic to the patient and (b) is easy to administer can provide IBD patients with a safer and practical solution to long-term control of autoimmune and inflammatory conditions.

In one aspect described herein is a method of treating an autoimmune or inflammatory condition in an individual in need thereof, the method comprising: a) systemic administration of a first inhibitor to the individual in need thereof, wherein the first inhibitor comprises an inhibitor of interleukin-23 (IL-23), an inhibitor of tumor necrosis factor alpha (TNF-alpha), or combinations thereof; and b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof; thereby treating the autoimmune or inflammatory condition in the individual in need thereof.

In some embodiments, the first inhibitor or the second inhibitor are substantially resistant to one or more proteases present in the intestinal tract or to one or more yeast proteases. In some embodiments, the systemic administration of the first inhibitor is inhalation, subcutaneous, intravenous, or oral. In some embodiments, the systemic administration of the first inhibitor is inhalation, subcutaneous, or intravenous. In some embodiments, the systemic administration of the first inhibitor is subcutaneous or intravenous. In some embodiments, the systemic administration of the first inhibitor is inhalation or intravenous. In some embodiments, the systemic administration of the first inhibitor is inhalation or subcutaneous. In some embodiments, the systemic administration of the first inhibitor is oral. In some embodiments, the systemic administration of the first inhibitor is by inhalation. In some embodiments, the systemic administration of the first inhibitor is subcutaneous. In some embodiments, the systemic administration of the first inhibitor is intravenous.

In some embodiments, the systemic administration of the first inhibitor occurs prior to the local administration of the second inhibitor. In some embodiments, the systemic administration of the first inhibitor occurs prior to the local administration of the second inhibitor by at least 24 hours. In some embodiments, the systemic administration of the first inhibitor occurs prior to the local administration of the second inhibitor by at least 2 days. In some embodiments, the systemic administration of the first inhibitor occurs prior to the local administration of the second inhibitor by at least 3 days. In some embodiments, the systemic administration of the first inhibitor occurs prior to the local administration of the second inhibitor by at least 7 days. In some embodiments, the systemic administration of the first inhibitor occurs prior to the local administration of the second inhibitor by at least 14 days. In some embodiments, the systemic administration of the first inhibitor occurs prior to the local administration of the second inhibitor by at least one month. In some embodiments, the systemic administration of the first inhibitor occurs prior to the local administration of the second inhibitor by at least two months. In some embodiments, the systemic administration of the first inhibitor occurs prior to the local administration of the second inhibitor by at least three months. In some embodiments, the systemic administration of the first inhibitor occurs prior to the local administration of the second inhibitor by at least four months.

In some embodiments, the local administration of the second inhibitor occurs after a clinical benefit is observed after systemic administration of the first inhibitor. In some embodiments, the clinical benefit comprises: reduction in the severity of diarrhea, reduction in the frequency of diarrhea, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

In some embodiments, the local administration of the second inhibitor is oral. In some embodiments, the first inhibitor comprises an inhibitor of IL-23. In some embodiments, the inhibitor of IL-23 comprises a small molecule inhibitor of IL-23. In some embodiments, the small molecule inhibitor of IL-23 comprises STA-5326 or peptide 2305. In some embodiments, the inhibitor of IL-23 comprises an antibody that binds IL-23 or an IL-23 binding fragment thereof. In some embodiments, the antibody that binds IL-23 or an IL-23 binding fragment thereof comprises risankizumab, guselkumab, tildrakizumab, briakinumab, brazikumab, mirikizumab, ustekinumab, or combinations thereof.

In some embodiments, the first inhibitor comprises an inhibitor of TNF-alpha. In some embodiments, the inhibitor of TNF-alpha comprises a small molecule inhibitor of TNF-alpha. In some embodiments, the small molecule inhibitor of TNF-alpha comprises SAR441566, TIM1, TIM1c, SPD-304, MYMD-1, or combinations thereof. In some embodiments, the inhibitor of TNF-alpha comprises an antibody that binds TNF-alpha or a TNF-alpha binding fragment thereof. In some embodiments, the antibody that binds TNF-alpha or a TNF-alpha binding fragment thereof comprises infliximab, adalimumab, certolizumab pegol, golimumab, or combinations thereof. In some embodiments, the inhibitor of TNF-alpha comprises a soluble TNF-alpha receptor. In some embodiments, the soluble TNF-alpha receptor comprises etanercept.

In some embodiments, the first inhibitor comprises an inhibitor of IL-23 and an inhibitor of TNF-alpha. In some embodiments, the inhibitor of IL-23 and an inhibitor of TNF-alpha are administered separately.

In some embodiments, the second inhibitor comprises an inhibitor of IL-23. In some embodiments, the inhibitor of IL-23 comprises an antibody that binds IL-23 or an IL-23 binding fragment thereof. In some embodiments, the antibody that binds IL-23 or an IL-23 binding fragment thereof comprises a VHH. In some embodiments, the VHH comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9. In some embodiments, the VHH comprises an amino acid sequence at least about 85%, 90%, 95%, 97%, 98%, or 99% identical to that set forth in any one of SEQ ID NOs: 10-12.

In some embodiments, the second inhibitor comprises an inhibitor of TNF-alpha. In some embodiments, the inhibitor of TNF-alpha comprises an antibody that binds TNF-alpha or a TNF-alpha binding fragment thereof. In some embodiments, the antibody that binds TNF-alpha or an TNF-alpha binding fragment thereof comprises a VHH. In some embodiments, the VHH comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the VHH comprises an amino acid sequence at least about 85%, 90%, 95%, 97%, 98%, or 99% identical to that set forth in any one of SEQ ID NOs: 118 or 125.

In some embodiments, the second inhibitor comprises a JAK inhibitor. In some embodiments, the JAK inhibitor comprises a small molecule JAK inhibitor of JAK1, JAK2, JAK3, TYK2, or combinations thereof. In some embodiments, the JAK inhibitor comprises an antibody or antibody binding fragment thereof that binds to JAK1, JAK2, JAK3, TYK2, or combinations thereof. In some embodiments, the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof. In some embodiments, the second inhibitor comprises a S1P modulator. In some embodiments, the S1P modulator is a small molecule S1P modulator In some embodiments, the small molecule S1P modulator can agonize or antagonize S1PR1, S1PR2, S1PR3, S1PR4, S1PR5, or combinations thereof. In some embodiments, the small molecule S1P modulator comprises fingolimod, ozanimod, siponimod, ponesimod, or combinations thereof.

In some embodiments, the second inhibitor comprises an inhibitor of TL1A. In some embodiments, the inhibitor of TL1A comprises a small molecule inhibitor of TL1A. In some embodiments, the inhibitor of TL1A comprises an antibody or antibody fragment thereof that binds to TL1A. In some embodiments, the antibody or antibody binding fragment thereof that binds to TL1A comprises PRA023, PF-06480605, or combinations thereof.

In some embodiments, the first inhibitor and the second inhibitor are administered separately. In some embodiments, the first inhibitor and the second inhibitor are administered simultaneously.

In some embodiments, the first inhibitor and the second inhibitor is an inhibitor of IL-23 and TNF-alpha. In some embodiments, the inhibitor of IL-23 and the inhibitor of TNF-alpha comprises a polypeptide that binds to both IL-23 and TNF-alpha. In some embodiments, the polypeptide that binds to both IL-23 and TNF-alpha comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID 10-12; and wherein the second binding region comprises an amino acid sequence at least about 85%, 90%, 95%, 97%, 98%, or 99% identical to that set forth in any one of SEQ ID NOs: 118 or 125. In some embodiments, the first binding region and the second binding region are coupled by a protease liable linker.

In some embodiments, the method further comprises administration of a third inhibitor wherein the third inhibitor comprises an inhibitor of IL-23, an inhibitor of TNF-alpha, a JAK inhibitor, a modulator of S1P, a TL1A inhibitor, or combinations thereof. In some embodiments, administration of the third inhibitor is local. In some embodiments, administration of the third inhibitor is oral.

In some embodiments, the local administration of the third inhibitor occurs prior to the local administration of the second inhibitor. In some embodiments, the local administration of the third inhibitor occurs prior to the local administration of the second inhibitor by at least 24 hours. In some embodiments, the local administration of the third inhibitor occurs prior to the local administration of the second inhibitor by at least 2 days. In some embodiments, the local administration of the third inhibitor occurs prior to the local administration of the second inhibitor by at least 3 days. In some embodiments, the local administration of the third inhibitor occurs prior to the local administration of the second inhibitor by at least 7 days. In some embodiments, the local administration of the third inhibitor occurs prior to the local administration of the second inhibitor by at least 14 days. In some embodiments, the local administration of the third inhibitor occurs prior to the local administration of the second inhibitor by at least one month. In some embodiments, the local administration of the third inhibitor occurs prior to the local administration of the second inhibitor by at least two months. In some embodiments, the local administration of the third inhibitor occurs prior to the local administration of the second inhibitor by at least three months. In some embodiments, the local administration of the third inhibitor occurs prior to the local administration of the second inhibitor by at least four months.

In some embodiments, the third inhibitor comprises an inhibitor of IL-23. In some embodiments, the inhibitor of IL-23 comprises an antibody that binds IL-23 or an IL-23 binding fragment thereof. In some embodiments, the antibody that binds IL-23 or an IL-23 binding fragment thereof comprises a VHH. In some embodiments, the VHH comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9. In some embodiments, the VHH comprises an amino acid sequence at least about 85%, 90%, 95%, 97%, 98%, or 99% identical to that set forth in any one of SEQ ID NOs: 10-12.

In some embodiments, the third inhibitor comprises an inhibitor of TNF-alpha. In some embodiments, the inhibitor of TNF-alpha comprises an antibody that binds TNF-alpha or a TNF-alpha binding fragment thereof. In some embodiments, the antibody that binds TNF-alpha or an TNF-alpha binding fragment thereof comprises a VHH. In some embodiments, the VHH comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the VHH comprises an amino acid sequence at least about 85%, 90%, 95%, 97%, 98%, or 99% identical to that set forth in any one of SEQ ID NOs: 118 or 125.

In some embodiments, the third inhibitor is an inhibitor of IL-23 and TNF-alpha. In some embodiments, the inhibitor of IL-23 and the inhibitor of TNF-alpha comprises a polypeptide that binds to both IL-23 and TNF-alpha. In some embodiments, the polypeptide that binds to both IL-23 and TNF-alpha comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125. In some embodiments, the first binding region and the second binding region are coupled by a protease liable linker.

In some embodiments, the third inhibitor comprises a JAK inhibitor. In some embodiments, the JAK inhibitor comprises a small molecule JAK inhibitor of JAK1, JAK2, JAK3, TYK2, or combinations thereof. In some embodiments, the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof. In some embodiments, the JAK inhibitor comprises an antibody or antibody fragment that binds to JAK1, JAK2, JAK3, TYK2, or combinations thereof.

In some embodiments, the third inhibitor comprises a S1P modulator. In some embodiments, the S1P modulator is a small molecule S1P modulator In some embodiments, the small molecule S1P modulator can agonize or antagonize S1PR1, S1PR2, S1PR3, S1PR4, S1PR5, or combinations thereof. In some embodiments, the small molecule S1P modulator comprises fingolimod, ozanimod, siponimod, ponesimod, or combinations thereof.

In some embodiments, the third inhibitor comprises an inhibitor of TL1A. In some embodiments, the inhibitor of TL1A comprises a small molecule inhibitor of TL1A. In some embodiments, the inhibitor of TL1A comprises an antibody or antibody fragment thereof that binds to TL1A. In some embodiments, the antibody or antibody binding fragment thereof that binds to TL1A comprises PRA023, PF-06480605, or combinations thereof.

In some embodiments, the autoimmune or inflammatory condition is an autoimmune and/or inflammatory condition of the gastrointestinal tract. In some embodiments, the autoimmune and/or inflammatory condition of the gastrointestinal tract comprise inflammatory bowel disease or Crohn's disease.

In one aspect described herein is a method of treating an autoimmune or inflammatory condition in an individual in need thereof, the method comprising: a) systemic administration of a first inhibitor to the individual in need thereof, wherein the first inhibitor comprises an inhibitor of interleukin-23 (IL-23) and/or an inhibitor of tumor necrosis factor alpha (TNF-alpha); and b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof; thereby treating the autoimmune or inflammatory condition in the individual in need thereof.

In some embodiments, the systemic administration of the first inhibitor is subcutaneous or intravenous. In some embodiments, the systemic administration of the first inhibitor is subcutaneous. In some embodiments, the systemic administration of the first inhibitor is intravenous.

In some embodiments, the systemic administration of the first inhibitor occurs prior to the local administration of the second inhibitor. In some embodiments, the systemic administration of the first inhibitor occurs prior to the local administration of the second inhibitor by at least 24 hours. In some embodiments, the systemic administration of the first inhibitor occurs prior to the local administration of the second inhibitor by at least 2 days. In some embodiments, the systemic administration of the first inhibitor occurs prior to the local administration of the second inhibitor by at least 3 days. In some embodiments, the systemic administration of the first inhibitor occurs prior to the local administration of the second inhibitor by at least 7 days. In some embodiments, the systemic administration of the first inhibitor occurs prior to the local administration of the second inhibitor by at least 14 days. In some embodiments, the systemic administration of the first inhibitor occurs prior to the local administration of the second inhibitor by at least one month. In some embodiments, the systemic administration of the first inhibitor occurs prior to the local administration of the second inhibitor by at least two months. In some embodiments, the systemic administration of the first inhibitor occurs prior to the local administration of the second inhibitor by at least three months. In some embodiments, the systemic administration of the first inhibitor occurs prior to the local administration of the second inhibitor by at least 4 months. In some embodiments, the local administration of the second inhibitor is oral.

In some embodiments, the first inhibitor comprises an inhibitor of IL-23. In some embodiments, the inhibitor of IL-23 comprises a small molecule inhibitor of IL-23. In some embodiments, the small molecule inhibitor of IL-23 comprises STA-5326 or peptide 2305. In some embodiments, the inhibitor of IL-23 comprises an antibody that binds IL-23 or an IL-23 binding fragment thereof. In some embodiments, the antibody that binds IL-23 or an IL-23 binding fragment thereof comprises risankizumab, guselkumab, tildrakizumab, briakinumab, brazikumab, mirikizumab, ustekinumab, or combinations thereof.

In some embodiments, the first inhibitor comprises an inhibitor of TNF-alpha. In some embodiments, the inhibitor of TNF-alpha comprises a small molecule inhibitor of TNF-alpha. In some embodiments, the small molecule inhibitor of TNF-alpha comprises SAR441566, TIM1, TIM1c, SPD-304, MYMD-1, or combinations thereof. In some embodiments, the inhibitor of TNF-alpha comprises an antibody that binds TNF-alpha or a TNF-alpha binding fragment thereof. In some embodiments, the antibody that binds TNF-alpha or a TNF-alpha binding fragment thereof comprises infliximab, adalimumab, certolizumab pegol, golimumab, or combinations thereof. In some embodiments, the inhibitor of TNF-alpha comprises a soluble TNF-alpha receptor. In some embodiments, the soluble TNF-alpha receptor comprises etanercept.

In some embodiments, the inhibitor of IL-23 and the inhibitor of TNF-alpha comprises a dual inhibitor of interleukin-23 (IL-23) and tumor necrosis factor alpha (TNF-alpha) comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125.

In some embodiments, the second inhibitor comprises a JAK inhibitor. In some embodiments, the JAK inhibitor comprises a small molecule JAK inhibitor of JAK1, JAK2, JAK3, TYK2, or combinations thereof. In some embodiments, the JAK inhibitor comprises an antibody or antibody binding fragment thereof that binds to JAK1, JAK2, JAK3, TYK2, or combinations thereof. In some embodiments, the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

In some embodiments, the second inhibitor comprises a S1P modulator. In some embodiments, the SP modulator is a small molecule S1P modulator In some embodiments, the small molecule S1P modulator can agonize or antagonize S1PR1, S1PR2, S1PR3, S1PR4, S1PR5, or combinations thereof. In some embodiments, the small molecule S1P modulator comprises fingolimod, ozanimod, siponimod, ponesimod, or combinations thereof. In some embodiments, the second inhibitor comprises an inhibitor of TL1A. In some embodiments, the inhibitor of TL1A comprises a small molecule inhibitor of TL1A. In some embodiments, the inhibitor of TL1A comprises an antibody or antibody fragment thereof that binds to TL1A. In some embodiments, the antibody or antibody binding fragment thereof that binds to TL1A comprises PRA023, PF-06480605, or combinations thereof.

In some embodiments, systemic administration of the first inhibitor is ended after 1 week, 2 weeks, 3 weeks, 4 weeks, 2 months, 3 months, or 4 months after the start of the systemic administration. In some embodiments, systemic administration of the first inhibitor is ended after a clinical benefit is observed after systemic administration of the first inhibitor. In some embodiments, the clinical benefit comprises: reduction in the severity of diarrhea, reduction in the frequency of diarrhea, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

In one aspect described herein is a method of treating an autoimmune and/or inflammatory condition in an individual in need thereof, the method comprising: a) systemic administration of a first inhibitor to the individual in need thereof, wherein the first inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof; and b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof; thereby treating the autoimmune and/or inflammatory condition in the individual in need thereof.

In some embodiments, the systemic administration of the first inhibitor is subcutaneous or intravenous. In some embodiments, the systemic administration of the first inhibitor is subcutaneous. In some embodiments, the systemic administration of the first inhibitor is intravenous.

In some embodiments, the systemic administration of the first inhibitor occurs prior to the local administration of the second inhibitor. In some embodiments, the systemic administration of the first inhibitor occurs prior to the local administration of the second inhibitor by at least 24 hours. In some embodiments, the systemic administration of the first inhibitor occurs prior to the local administration of the second inhibitor by at least 2 days. In some embodiments, the systemic administration of the first inhibitor occurs prior to the local administration of the second inhibitor by at least 3 days. In some embodiments, the systemic administration of the first inhibitor occurs prior to the local administration of the second inhibitor by at least 7 days. In some embodiments, the systemic administration of the first inhibitor occurs prior to the local administration of the second inhibitor by at least 14 days. In some embodiments, the systemic administration of the first inhibitor occurs prior to the local administration of the second inhibitor by at least one month. In some embodiments, the systemic administration of the first inhibitor occurs prior to the local administration of the second inhibitor by at least two months. In some embodiments, the systemic administration of the first inhibitor occurs prior to the local administration of the second inhibitor by at least three months. In some embodiments, systemic administration of the first inhibitor occurs prior to the local administration of the second inhibitor by at least 4 months. In some embodiments, systemic administration of the first inhibitor occurs prior to the local administration of the second inhibitor by at least 12 months. In some embodiments, the local administration of the second inhibitor is oral.

In some embodiments, the first inhibitor comprises an inhibitor of IL-23. In some embodiments, the inhibitor of IL-23 comprises a small molecule inhibitor of IL-23. In some embodiments, the small molecule inhibitor of IL-23 comprises a peptide In some embodiments, the inhibitor of IL-23 comprises a miniprotein. In some embodiments, the miniprotein comprises an IL-23R minibinder. In some embodiments, the small molecule inhibitor of IL-23 comprises STA-5326, peptide 2305, combinations thereof. In some embodiments, the inhibitor of IL-23 comprises an antibody that binds IL-23 or an IL-23 binding fragment thereof. In some embodiments, the antibody that binds IL-23 or an IL-23 binding fragment thereof comprises risankizumab, guselkumab, tildrakizumab, briakinumab, brazikumab, mirikizumab, ustekinumab, or combinations thereof.

In some embodiments, the first inhibitor comprises an inhibitor of TNF-alpha. In some embodiments, the inhibitor of TNF-alpha comprises a small molecule inhibitor of TNF-alpha. In some embodiments, the small molecule inhibitor of TNF-alpha comprises SAR441566, TIM1, TIM1c, SPD-304, MYMD-1, or combinations thereof. In some embodiments, the inhibitor of TNF-alpha comprises an antibody that binds TNF-alpha or a TNF-alpha binding fragment thereof. In some embodiments, the antibody that binds TNF-alpha or a TNF-alpha binding fragment thereof comprises infliximab, adalimumab, certolizumab pegol, golimumab, or combinations thereof. In some embodiments, the inhibitor of TNF-alpha comprises a soluble TNF-alpha receptor. In some embodiments, the soluble TNF-alpha receptor comprises etanercept.

In some embodiments, the inhibitor of IL-23 and the inhibitor of TNF-alpha comprises a dual inhibitor of interleukin-23 (IL-23) and tumor necrosis factor alpha (TNF-alpha) comprises a first binding region that binds to IL-23 and a second binding region that binds to TNF-alpha, wherein the first binding region that binds to IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds to TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125.

In some embodiments, the second inhibitor comprises a JAK inhibitor. In some embodiments, the JAK inhibitor comprises a small molecule JAK inhibitor of JAK1, JAK2, JAK3, TYK2, or combinations thereof. In some embodiments, the JAK inhibitor comprises an antibody or antibody binding fragment thereof that binds to JAK1, JAK2, JAK3, TYK2, or combinations thereof. In some embodiments, the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

In some embodiments, the second inhibitor comprises a TYK2 inhibitor. In some embodiments, the TYK2 inhibitor comprises TAK-279, VTX958, ESK-001, or combinations thereof.

In some embodiments, the second inhibitor comprises a S1P modulator. In some embodiments, the S1P modulator is a small molecule S1P modulator. In some embodiments, the small molecule S1P modulator can agonize or antagonize S1PR1, S1PR2, S1PR3, S1PR4, S1PR5, or combinations thereof. In some embodiments, the small molecule S1P modulator comprises fingolimod, ozanimod, siponimod, ponesimod, etrasimod, tamuzimod, amiselimod or combinations thereof. In some embodiments, the second inhibitor comprises an inhibitor of TL1A. In some embodiments, the inhibitor of TL1A comprises a small molecule inhibitor of TL1A. In some embodiments, the inhibitor of TL1A comprises an antibody or antibody fragment thereof that binds to TL1A. In some embodiments, the antibody or antibody binding fragment thereof that binds to TL1A comprises PRA023, PF-06480605, TEV-48574, or combinations thereof.

In some embodiments, the local administration of the second inhibitor occurs after a clinical benefit is observed after systemic administration of the first inhibitor. In some embodiments, the clinical benefit comprises: reduction in the severity of diarrhea, reduction in the frequency of diarrhea, reduction in the frequency of urgency, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping reduction in the frequency of abdominal pain, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

In some embodiments, the autoimmune or inflammatory condition is an autoimmune or inflammatory condition of the gastrointestinal tract. In some embodiments, the autoimmune or inflammatory condition of the gastrointestinal tract comprise inflammatory bowel disease, Crohn's disease, or ulcerative colitis.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A illustrates a schematic of a single-domain heavy chain antibody (VHH) comprising four framework regions (FRs) and three complementarity determining regions (CDRs).

FIG. 1B illustrates a schematic of a construct comprising two identical VHHs joined by a linker, with each VHH comprising four framework regions (FRs) and three complementarity determining regions (CDRs).

FIG. 1C illustrates a schematic of a construct comprising two distinct VHHs joined by a linker, with each VHH comprising four framework regions (FRs) and three complementarity determining regions (CDRs).

FIG. 2 illustrates a non-limiting example of a treatment schedule described herein in which the arrows on the figure indicate the start of a treatment regimen.

FIG. 3 illustrates a non-limiting example of a treatment schedule described herein in which the arrows on the figure indicate the start of a treatment regimen.

FIG. 4 illustrates a non-limiting example of a treatment schedule described herein in which the arrows on the figure indicate the start of a treatment regimen.

FIG. 5 demonstrates that a bispecific anti-TNF-alpha/anti-IL-23 domain antibody (“TNF-alpha/IL-23 dual inhibitor”) and its liberated monomer arms can retain full anti-TNF-alpha and anti-IL-23 activity after exposure to trypsin and high levels of anti-TNF-alpha and anti-IL-23 activity after exposure to human fecal supernatant. FIG. 5A shows cleavage of the central lysine in TNF-alpha/IL-23 dual inhibitor. TNF-alpha/IL-23 dual inhibitor was incubated at 37° C. with 1/1,000 diluted faecal supernatant (HFS). Samples were taken for SDS-PAGE analysis at selected time intervals, shown in minutes (horizontal numbers). Equal volumes were loaded per lane. ‘St’ is undigested TNF-alpha/IL-23 dual inhibitor standard. L=protein standard EZ-Run Prestained Ladder. MW=Molecular weight in kDa (vertical numbers). FIG. 5B shows inhibition curves assessed by ELISAs wherein binding molecules competed with biotinylated adalimumab (anti-TNF-alpha IgG1). TNF-alpha/IL-23 dual inhibitor and the trypsin-liberated TNF-alpha monomer arm (“liberated-TNF”) were tested alongside the anti-TNF-alpha single domain antibody (“TNF-alpha inhibitor”) parent in a biotinylated adalimumab competition ELISA, where biotinylated adalimumab alone was used as a control. FIG. 5C shows inhibition curves assessed by ELISAs wherein binding molecules competed interrupted IL-23/IL-23-receptor binding. TNF-alpha/IL-23 dual inhibitor and the trypsin-liberated IL-23 monomer arm (“liberated-IL-23”) were tested alongside the anti-IL-23 single domain antibody (“IL-23 inhibitor”) parent in the IL-23/IL-23R ELISA, where IL-23 alone was used as a control. FIG. 5D shows functional activity retention of TNF-alpha/IL-23 dual inhibitor and its cleavage products after exposure to HFS. TNF-alpha/IL-23 dual inhibitor and the parent monomers, TNF-alpha inhibitor and IL-23 inhibitor, were incubated in pooled human fecal supernatant (HFS) for 4 hours. Time 0 hour and 4 hour samples were compared for anti-TNF-alpha activity in the biotinylated adalimumab assay (TNF-alpha inhibitor and TNF-alpha/IL-23 dual inhibitor) or anti-IL-23 activity in the IL-23/IL-23R ELISA (IL-23 inhibitor and TNF-alpha/IL-23 dual inhibitor). The remaining activity in each sample at 4 hours was calculated as a survival percentage against the 0 hour time point. Error bars+/−SD. N=3. FIG. 5E and FIG. 5F show TNF-alpha inhibitor- and IL-23-inhibitor-mediated inhibition, alone and in combination, of phosphorylation signals in ulcerative colitis (UC) colonic human biopsies. Biopsies were collected from four different UC patients and incubated for 24 hours with single domain antibody treatments (Control (ID2A) 225 nM; TNF-alpha inhibitor 75 nM; IL-23 inhibitor 150 nM; or TNF-alpha inhibitor 75 nM+IL-23 inhibitor 150 nM combined) and analyzed for the extent of phosphorylation of tyrosine kinase receptors and signalling proteins that can be increased in inflamed intestinal tissue. Heatmap shadings were applied relative to the averaged signal of each phosphoprotein in the final array data set. Light shading indicates strong phosphorylation signals; dark shading indicates weak phosphorylation signals.

FIG. 6 shows the pharmacokinetics of TNF-alpha/IL-23 dual inhibitor, the liberated anti-TNF-alpha monomer arm (“liberated-TNF”), and the liberated anti-IL-23 monomer arm (“liberated-IL-23”). 10 non-human primates (NHPs) were dosed orally twice a day for 42 days (135 mg/dose, 270 mg per day total). On Day 42, Fecal samples of cynomolgus monkeys were collected at 6-hour timepoints following the first daily dose of TNF-alpha/IL-23 dual inhibitor for 10 animals during the first 24 hours after dosing and for 4 animals for an additional 24 hours (two 12-hour sample collection time points). 6 animals were given a third oral dose of TNF-alpha/IL-23 dual inhibitor at the 24-hour time point and culled 4 hours after this final dose for intestinal analysis. Intact TNF-alpha/IL-23 dual inhibitor levels were below the assay lower limit of quantitation (245 nM) in all intestinal samples, therefore fecal samples were not analyzed for intact TNF-alpha/IL-23 dual inhibitor. FIG. 6A shows the concentration of liberated-TNF and liberated-IL-23 in fecal samples at various timepoints. FIG. 6B shows the concentration of liberated-TNF and liberated-IL-23 in intestinal content of cynomolgus monkeys that were culled 4 hours after the third dose. Horizontal lines indicate the average lower limit of quantitation (LLOQ) across all assay plates (liberated-TNF=0.012 μM, dotted; liberated-IL-23=0.039 μM, dashed); black lines in each group indicate mean values; numbers located below the x axes indicate the number of samples positive for each analyte vs the total number of samples collected for the indicated fecal time point or intestinal sample type; each animal is represented as a unique symbol; and symbols are consistent across both panels.

FIG. 7 shows the serum concentration of TNF-alpha/IL-23 dual inhibitor, liberated-TNF, and liberated-IL-23 after intravenous administration in NHPs (n=3 per group). The serum concentration of each molecule was measured for the first 36 hours post-administration by ELISA.

### DETAILED DESCRIPTION

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount”, “effective dose”, “effective amount”, or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity.

The term “antibody” herein is used in the broadest sense and includes monoclonal antibodies, bispecific antibodies, and includes intact antibodies and functional (antigen-binding) antibody fragments thereof, including fragment antigen binding (Fab) fragments, F(ab′) 2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody, VHH) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The term “small molecule” typically refer to organic, inorganic or organometallic compounds having a molecular weight of less than about 2000 Daltons.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Antibodies and Antibody-Derived Binding Moieties

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs or single heavy chain variable domains.

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets with high affinity (e.g., with a dissociation constant (K_{D}) less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides, or comprise separate domains of the same polypeptide. For example, two distinct binding moieties or a heavy-chain/light-chain pair. A number of strategies may be used to covalently link molecules together. Linkers described herein may be utilized to join a light chain variable region and a heavy chain variable region in an scFv molecule; or may be used to tether an scFv or other antigen binding fragment on the N- or C-terminus of an antibody heavy chain; or the N- or C-terminus of a light chain to create a bispecific or multispecific binding molecule. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length may be used. Useful linkers include glycine-serine polymers, including for example (GS)n, (GSGGS)n (SEQ ID NO: 303), (GGGGS)n (SEQ ID NO: 304), and (GGGS)n (SEQ ID NO: 305), where n is an integer of at least one, glycine-alanine polymers, alanine-serine polymers, and other flexible linkers. Exemplary, linkers for linking antibody fragments or single chain variable fragments can include AAEPKSS (SEQ ID NO: 306), AAEPKSSDKTHTCPPCP (SEQ ID NO: 307), GGGG (SEQ ID NO: 308), or GGGGDKTHTCPPCP (SEQ ID NO: 309). Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5^{th} Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6^{th} ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

An exception to conventional antibody structure can found in sera of camelids and cartilaginous fish (e.g., sharks). In addition to conventional antibodies, these sera possess special IgG antibodies. These IgG antibodies, known as heavy-chain antibodies (HCAbs), are devoid of the L chain polypeptide and lack the first constant domain (CH1). At its N-terminal region, the H chain of the homodimeric protein contains a dedicated immunoglobulin chain variable domain, referred to as the VHH for camelids and vNAR for sharks, which serves to associate with its cognate antigen (Muyldermans 2013 Annu Rev Biochem 82:775-797, Hamers-Casterman et al 1993 Nature 363(6428):446-448, Muyldermans et al 1994 Protein Eng 7(9):1129-1135, Könning et al 2017 Curr Opin Struct Biol 45:10-16 herein incorporated by reference in their entirety).

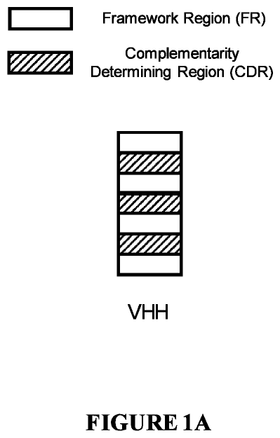

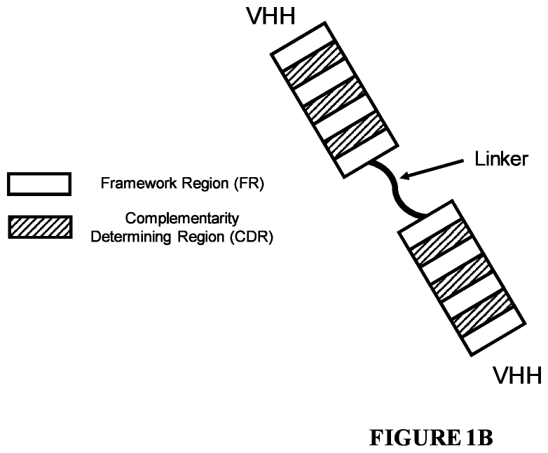

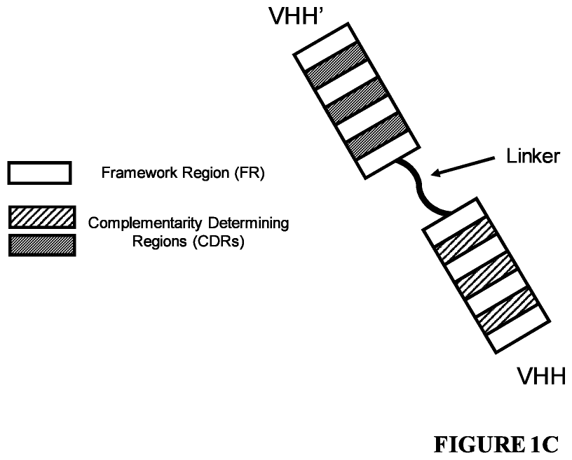

VHHs are single-domain antibodies that comprise a single heavy chain variable domain. Similar to the variable domains from conventional antibodies, VHHs comprise four FRs and three CDRs (FIG. 1A). Unlike conventional antibodies, VHHs do not have nor require a light chain variable domain to pair with its heavy chain variable domain for antigen binding to occur. In some embodiments, the VHHs described herein are stable throughout recombinant production and in vivo delivery. In some embodiments, the VHHs described herein are resistant to cleavage by recombinant host organism (e.g., yeast) proteases and by proteases present in the GI tract.

VHHs can be used to construct multivalent and/or multispecific polypeptides. In some embodiments, a polypeptide described herein can comprise at least two identical VHHs (FIG. 1B). In some embodiments, a polypeptide described herein can comprise at least two distinct VHHs (FIG. 1C). A multivalent polypeptide comprises two or more binding polypeptides (e.g., VHHs) which presents two or more sites at which binding to one or more antigens can occur. A multispecific polypeptide comprises two or more distinct binding polypeptides (e.g., VHHs) which present two or more sites at which either (a) binding to two or more distinct antigens can occur or (b) binding to two or more distinct epitopes on the same antigen can occur.

Multivalent and/or multispecific polypeptides described herein can incorporate linkers to covalently join functional domains (e.g., VHHs) together to act as one molecule throughout the recombinant production process and/or in vivo delivery. In some embodiments, the linkers described herein can be stable and resistant to cleavage by recombinant host organism (e.g., yeast) proteases as well as proteases present in the GI tract. In some embodiments, the linkers described herein can be labile. In some embodiments, the labile peptide linker can be engineered such that it resists cleavage by proteases to a desired extent and/or is only cleaved upon exposure to a specific area of the intestinal tract. This can be achieved according to one embodiment of the invention by incorporating shielding residues into the labile peptide linker flanking the labile site(s). Shielding residues flank the labile site(s) of the labile peptide linker and reduce the lability thereof. Cleavage resistance can also be increased by positioning the labile site(s) closer to or at the periphery of the labile peptide linker. This concept is referred to as a “shielded labile site” and provides controlled lability. In a further embodiment of the invention, the labile peptide linker can be engineered such that it is highly labile to cleavage by intestinal tract proteases, thereby quickly releasing the constituent first and second polypeptides of the construct after oral administration. This is achieved by incorporating one or more labile sites into the labile peptide linker such that the labile site is exposed for proteolysis, for example by positioning the labile site(s) substantially centrally in the labile peptide linker and/or by the labile site not being shielded substantially by flanking residues. This concept is referred to as a “non-shielded labile site”.

In some embodiments, an antibody or antibody-derived binding moiety provided herein has a K_{D} of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 1 μM or less (e.g., 10⁻⁸ M or less, e.g., from 10⁻⁸ M to 10⁻¹² M, e.g., from 10⁻⁹ M to 10⁻¹² M) for the antibody target. In some embodiments, an antibody or antibody-derived binding moiety provided herein has a K_{D} of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 1 μM or greater (e.g., 10⁻¹² M or greater, e.g., from 10⁻⁸ M to 10⁻¹² M, e.g., from 10⁻⁹ M to 10⁻¹² M) for the antibody target. K_{D} can be measured by any suitable assay. In certain embodiments, K_{D} can be measured using surface plasmon resonance assays (e.g., using a BIACORE®-2000, a BIACORE®-3000 or Octet).

The antibody or antibody-derived binding moiety can be an IL-23 or an IL-23 receptor targeting antibody. In some embodiments, the IL-23-specific polypeptide of the invention can bind to IL-23 with a K_{D} of 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. In some embodiments, the polypeptide of the invention can bind to IL-23 with a K_{D} of 10⁻⁶ M or greater, 10⁻⁷ M or greater, 10⁻⁸ M or greater, 10⁻⁹ M or greater, 10⁻10 M or greater, 10⁻¹¹ M or greater, or 10⁻¹²M or greater. In some embodiments, the polypeptide of the invention can bind to IL-23 with a K_{D} of 10⁻⁶ M to 10⁻¹² M, 10⁻⁷ to 10⁻¹² M, 10⁻⁸ to 10⁻¹² M, or 10⁻⁹ to 10⁻¹² M.

The antibody or antibody-derived binding moiety can be a TNF-alpha or a TNF receptor targeting antibody. In some embodiments, the polypeptide of the invention can bind to TNF-alpha with a K_{D} of 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. In some embodiments, the polypeptide of the invention can bind to TNF-alpha with a K_{D} of 10⁻⁶ M or greater, 10⁻⁷ M or greater, 10⁻⁸ M or greater, 10⁻⁹ M or greater, 10⁻¹⁰ M or greater, 10⁻¹¹ M greater less, and 10⁻¹²M or greater. In some embodiments, the polypeptide of the invention can bind to TNF-alpha with a K_{D} of 10⁻⁶ M to 10⁻¹² M, 10⁻⁷ to 10⁻¹²M, 10⁻⁸ to 10⁻12 M, or 10⁻⁹ to 10⁻¹² M.

### Miniproteins and Minibinders

Miniproteins are small proteins that encompass a wide range of protein scaffolds. Miniproteins may be characterized in part by their relatively small size (such as having a molecular weight of less than 10,000 daltons), stability, and their ability to potentiate biological responses. In certain instances, miniproteins may bind to targets at low picomolar affinities. In certain instances, miniproteins may be used in therapeutic contexts. Miniproteins may be computationally designed de novo. In some instances, miniproteins can bind to specific targets of interest. In some instances, miniprotein binding to a specific target of interest can agonize or antagonize biological pathways. For example, a miniprotein may be an IL-23R minibinder or an IL-17 minibinder. In some instances, IL-23R or IL-17 minibinders may be used in the prevention or treatment of IL-23- or IL-17-associated conditions.

Autoimmune and/or Inflammatory Diseases

Autoimmune diseases develop when the immune system responds adversely to normal body tissues. Autoimmune disorders may result in damage to body tissues, abnormal organ growth and/or changes in organ function. The disorder may affect only one organ or tissue type or may affect multiple organs and tissues. Organs and tissues commonly affected by autoimmune disorders include blood components such as red blood cells, blood vessels, connective tissues, endocrine glands such as the thyroid or pancreas, muscles, joints and skin An inflammatory disease is a disease characterized by inflammation. Many inflammatory diseases are autoimmune diseases and vice-versa.

Autoimmune Diseases and/or Inflammatory Diseases of the GI Tract

A non-limiting list of other autoimmune and inflammatory diseases afflicting the GI tract include GI cancers, colitis, microscopic colitis, celiac disease, mucositis, pouchitis, or gastritis. A non-limiting list of GI cancers include anal cancer, bile duct cancer, colon cancer, esophageal cancer, small intestine cancer, and gastric cancer. The chronic inflammatory bowel diseases, Crohn's disease and ulcerative colitis, are examples of autoimmune and inflammatory diseases of the GI tract (Hendrickson et al 2002 Clin Microbiol Rev 15(1):79-94, herein incorporated by reference in its entirety). Ulcerative colitis is a condition where the inflammatory response and morphologic changes remain confined to the colon. The rectum is involved in 95% of patients. Inflammation is largely limited to the mucosa and consists of continuous involvement of variable severity with ulceration, edema, and hemorrhage along the length of the colon (Hendrickson et al 2002 Clin. Microbiol Rev 15(1):79-94, herein incorporated by reference in its entirety). Ulcerative colitis is a presently incurable life-long GI disease, and is usually manifested by the presence of blood and mucus mixed with stool, along with lower abdominal cramping which is most severe during the passage of bowel movements. Clinically, the presence of diarrhea with blood and mucus differentiates ulcerative colitis from irritable bowel syndrome, in which blood is absent.

Crohn's disease, also known as Crohn disease, Crohn syndrome and regional enteritis, is a type of inflammatory bowel disease causing a wide variety of symptoms. It primarily causes abdominal pain, diarrhea, vomiting and/or weight loss but may also cause complications outside GI tract such as anemia, skin rashes, arthritis, inflammation of the eye, tiredness, and lack of concentration (Baumgart et al 2012 The Lancet 380(9853):1590-605, herein incorporated by reference in its entirety). Crohn's disease is a presently incurable life-long GI disease that can affect any portion of the GI tract, and is difficult to control with conventional therapies. Unlike ulcerative colitis, the presentation of Crohn's disease is usually subtle, which leads to a later diagnosis. Factors such as the location, extent, and severity of involvement determine the extent of GI symptoms. Patients who have ileocolonic involvement usually have postprandial abdominal pain, with tenderness in the right lower quadrant and an occasional inflammatory mass. Symptoms associated with gastroduodenal Crohn's disease include early satiety, nausea, emesis, epigastric pain, or dysphagia. Perianal disease is common, along with anal tags, deep anal fissures, and fistulae (Hendrickson et al 2002 Clin Microbiol Rev 15(1):79-94, herein incorporated by reference in its entirety).

TNF-Alpha and IL-23 in Autoimmune Diseases and/or Inflammatory Diseases

Autoimmune and/or inflammatory diseases are characterized by aberrant activation of pro-inflammatory pathways. While the root pathology of autoimmune and/or inflammatory diseases remain to be fully elucidated, therapeutic strategies that aim to dampen pro-inflammatory pathways can provide sustained relief or induce remission to patients suffering from autoimmune and/or inflammatory diseases.

TNF-alpha (UniProt ID: P01375, www.uniprot.org/uniprotkb/P01375/entry) is a pleiotropic cytokine that is involved in a variety of pro-inflammatory cellular processes and is also implicated in the pathogenesis of autoimmune and/or inflammatory diseases. TNF alpha is a homo-trimeric protein that is primarily secreted by immune cell types, including natural killer (NK) cells, T cells, macrophages, and monocytes; and TNF-alpha can also be expressed as a transmembrane protein. Both transmembrane and soluble forms can bind to tumor necrosis factor receptor 1 (TNFR1, also known as p55) and tumor necrosis factor receptor 2 (TNFR2, also known as p75) to induce downstream signaling pathways, including nuclear factor kappa-light-chain-enhancer of activated B cells (NF-κB) and/or mitogen-activated protein kinase (MAPK) signaling.

IL-23 is a heterodimeric pro-inflammatory cytokine, comprising an IL-23A subunit (also known as IL-23p19; UniProt ID: Q9NPF7, www.uniprot.org/uniprotkb/Q9NPF7/entry) that shares an IL-12B subunit (also known as IL-12/23p40, UniProt ID: P29460, www.uniprot.org/uniprotkb/P29460/entry) with IL-12, that is implicated in the pathogenesis of autoimmune and/or inflammatory diseases. IL-23 is primarily secreted by immune cell types, such as activated macrophages, monocytes, or dendritic cells (DCs), can promote recruitment and activation of immune cells, such as macrophages and granulocytes, and is implicated in the maintenance and expansion of a pro-inflammatory T helper 17 (Tₕ17) cells. IL-23 signaling is mediated by binding of the IL-23 A subunit (also known as IL-23p19) to the IL-23 receptor (IL-23R) and the IL-12B subunit to IL-12 receptor beta 1 (IL-12Rβ1), thereby inducing JAK-mediated signal transducer and activator of transcription (STAT) signaling. In particular, IL-23 binding can recruit of JAK2 and TYK2 for the induction of STAT3 and/or STAT4 signaling pathways.

JAKs in Autoimmune Diseases and/or Inflammatory Diseases

JAK is a family of non-receptor tyrosine kinases that participates in signal transduction for biomolecules, such as cytokines and growth factors, through the JAK/STAT pathway. The JAK family comprise four JAK proteins: JAK1, JAK2, JAK3, and TYK2. JAKs, in combination with STAT proteins (STAT1, STAT2, STAT3, STAT4, STAT5A, STAT5B, and STAT6), participate in signal transduction from cell surface receptors to the cell nucleus. The JAK family comprise four JAK proteins: JAK1, JAK2, JAK3, and TYK2. The JAK/STAT pathway plays pleiotropic roles and at least 50 cytokines or growth factors signal via the JAK/STAT pathway (Darnell Jr. 1997 Science 277(5332):1630-5, herein incorporated by reference in its entirety). JAKs are commonly associated with the cytoplasmic domain of cell surface receptors. Canonically, ligand-induced activation of cell surface receptors facilitate receptor dimerization, thereby bringing JAKs on different receptor chains within close proximity. This enables JAK transphosphorylation which in turn activates the JAKs. Activated JAKs can phosphorylate the cytoplasmic domain of the cell surface receptor, which in turn facilitates recruitment of STAT proteins to the cytoplasmic domain of the cell surface receptor by providing a docking site for STAT proteins. Activated JAKs can subsequently phosphorylate docked STAT proteins. Phosphorylated STAT proteins can dissociate from the cell surface receptor and form homodimers or heterodimers. Phosphorylated STAT dimers can then translocate into the nucleus to regulate gene expression. While the root pathology of autoimmune and/or inflammatory diseases remain to be fully elucidated, dysregulated JAK/STAT signaling has been implicated in the pathogenesis of autoimmune and/or inflammatory conditions.

S1P in Autoimmune Diseases and/or Inflammatory Diseases

S1P is a sphingolipid signaling molecule that binds to and activates S1P receptors (S1PRs), which include S1PR1, S1PR2, S1PR3, S1PR4, and S1PR5 (Baeyens and Schwab 2020 Annual Review of Immunology 38:759-84, herein incorporated by reference in its entirety). Both S1P and S1PRs are ubiquitously expressed across human cell types, and the S1P-S1PR axis plays pleiotropic roles across diverse cell functions. Notably, the S1P-S1PR axis is involved with orchestrating adaptive and innate immune cell trafficking and responses, including lymphocyte egress from lymphoid tissue. S1P is present at low concentrations within lymphoid tissue but present at relatively higher concentrations in circulation (e.g., blood). S1PR-expressing lymphocytes can egress from lymphoid tissue and enter circulation in response to the S1P concentration gradient. S1P binding to S1PR can induce internalization of the bound S1P-S1PR complex, thereby reducing S1PR surface density and S1P concentration gradient-induced lymphocyte migration. While the root pathology of autoimmune and/or inflammatory diseases remain to be fully elucidated, the S1P-S1PR axis has been implicated in the pathogenesis of autoimmune and/or inflammatory conditions.

TL1A in Autoimmune and/or Inflammatory Diseases

TL1A (https://www.uniprot.org/uniprotkb/095150/entry) is a member of the TNF family and contributes to inflammatory responses and adaptive and innate immune homeostasis through binding to its cognate receptor death receptor 3 (DR3; Xu 2022 Frontiers in Immunology 13:891328, herein incorporated by reference in its entirety). TL1A can also bind to decoy receptor 3. TL1A is expressed in a variety of immune cells, such as macrophages, monocytes, and T cells, as well as non-immune cells such as endothelial cells. TL1A can be expressed in a membrane-bound form and can form stable TL1A trimers. TL1A can also exist in soluble form through alternative splicing or enzymatic conversion. TL1A binding to cognate receptor DR3 drives pro-inflammatory gene expression through NF-κB and MAPK signaling. Both membrane-bound and soluble forms of TL1A can activate signaling through DR3. While the root pathology of autoimmune and/or inflammatory diseases remain to be fully elucidated, TL1A has been implicated in the pathogenesis of autoimmune and/or inflammatory conditions.

### Inhibitors of IL-23

Inhibitors of IL-23 described herein can antagonize IL-23-induced cellular processes, which include pro-inflammatory pathways. In some embodiments, inhibitors of IL-23 described herein can bind to the IL-23 A subunit (also known as IL-23p19) of IL-23. In some embodiments, inhibitors of IL-23 described herein can bind to IL-23R. In some embodiments, inhibitors of IL-23 described herein can block binding of the IL-23A subunit to IL-23R. In some embodiments, inhibitors of IL-23 described herein can block binding of IL-23 to IL-23R. In some embodiments, inhibitors of IL-23 described herein can bind to the IL-12B subunit (also known as IL-12/23p40) of IL-23. In some embodiments, inhibitors of IL-23 described herein can bind to IL-12Rβ1. In some embodiments, inhibitors of IL-23 described herein can block binding of the IL-12B subunit to IL-12Rβ1. In some embodiments, inhibitors of IL-23 described herein can block binding of IL-23 to IL-12Rβ1. In some embodiments, inhibitors of IL-23 described herein can block binding of IL-12 to IL-12Rβ1. In some embodiments, inhibitors of IL-23 described herein can block signaling pathways activated in response to IL-12. In some embodiments, inhibitors of IL-23 described herein can block signaling pathways activated in response to IL-23. In some embodiments, inhibitors of IL-23 described herein can inhibit JAK/STAT signaling. In some embodiments, inhibitors of IL-23 described herein can inhibit STAT3 signaling, STAT4 signaling, or combinations thereof. In some embodiments, inhibitors of IL-23 described herein can inhibit cellular processes downstream of STAT3 signaling, STAT4 signaling, or combinations thereof. In some embodiments, inhibitors of IL-23 described herein can inhibit expression of pro-inflammatory genes.

In some embodiments, the inhibitor of IL-23 comprises a small molecule inhibitor of IL-23. In some embodiments, the small molecule inhibitor of IL-23 comprises a peptide. In some embodiments, the small molecule inhibitor of IL-23 comprises STA-5326, peptide 2305, or combinations thereof.

In some embodiments, the inhibitor of IL-23 comprises a miniprotein. In some embodiments, the miniprotein comprises an IL-23R minibinder. In some embodiments, the miniprotein comprises JNJ-2113.

In some embodiments, the inhibitor of IL-23 comprises an antibody that binds IL-23 or an IL-23 binding fragment thereof. In some embodiments, the antibody that binds IL-23 or an IL-23 binding fragment thereof comprises risankizumab, guselkumab, tildrakizumab, briakinumab, brazikumab, mirikizumab, ustekinumab, or combinations thereof.

In some embodiments, the antibody that binds to IL-23 or an IL-23 binding fragment thereof comprises a VHH. In some embodiments, the IL-23 binding VHH comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9. In some embodiments, the IL-23 binding VHH comprises an amino acid sequence at least about 85%, 90%, 95%, 97%, 98%, or 99% identical to that set forth in any one of SEQ ID NOs: 10-12.

### Inhibitors of TNF-Alpha

Inhibitors of TNF-alpha described herein can antagonize TNF-alpha-induced cellular processes, including pro-inflammatory pathways. In some embodiments, inhibitors of TNF-alpha described herein can bind to the soluble form of TNF-alpha. In some embodiments, inhibitors of TNF-alpha described herein can bind to transmembrane forms of TNF-alpha. In some embodiments, inhibitors of TNF-alpha described herein can bind to both soluble forms of TNF-alpha, transmembrane forms of TNF-alpha, or combinations thereof. In some embodiments, inhibitors of TNF-alpha described herein can also bind to TNFR1. In some embodiments, inhibitors of TNF-alpha described herein can also bind to TNFR2. In some embodiments, inhibitors of TNF-alpha described herein can also bind to TNFR1, TNFR2, or combinations thereof. In some embodiments, inhibitors of TNF-alpha described herein can bind to both soluble and transmembrane forms of TNF-alpha. In some embodiments, inhibitors of TNF-alpha described herein can block binding of the soluble form of TNF-alpha to TNFR1. In some embodiments, inhibitors of TNF-alpha described herein can block binding of the soluble form of TNF-alpha to TNFR2. In some embodiments, inhibitors of TNF-alpha described herein can block binding of the transmembrane form of TNF-alpha to TNFR1. In some embodiments, inhibitors of TNF-alpha described herein can block binding of the transmembrane form of TNF-alpha to TNFR2. In some embodiments, inhibitors of TNF-alpha described herein can block binding of both the soluble and transmembrane form of TNF-alpha to TNFR1. In some embodiments, inhibitors of TNF-alpha described herein can block binding of both the soluble and transmembrane form of TNF-alpha to TNFR2. In some embodiments, inhibitors of TNF-alpha described herein can block binding of both the soluble and transmembrane form of TNF-alpha to TNFR1, TNRFR2, or combinations thereof. In some embodiments, inhibitors of TNF-alpha described herein can block signaling pathways activated in response to TNF-alpha. In some embodiments, inhibitors of TNF-alpha described herein can inhibit NF-κB and/or MAPK signaling. In some embodiments, inhibitors of TNF-alpha described herein can inhibit cellular processes downstream of NF-κB and/or MAPK signaling. In some embodiments, inhibitors of TNF-alpha described herein can inhibit expression of pro-inflammatory genes.

In some embodiments, the inhibitor of TNF-alpha comprises a small molecule inhibitor of TNF-alpha. In some embodiments, the small molecule inhibitor of TNF-alpha comprises SAR441566, TIM1, TIM1c, SPD-304, MYMD-1, or combinations thereof.

In some embodiments, the inhibitor of TNF-alpha comprises an antibody that binds TNF-alpha or a TNF-alpha binding fragment thereof. In some embodiments, the antibody that binds TNF-alpha or TNF-alpha binding fragment thereof comprises infliximab, adalimumab, certolizumab pegol, golimumab, or combinations thereof. In some embodiments, the inhibitor of TNF-alpha comprises a soluble TNF-alpha receptor. In some embodiments, the soluble TNF-alpha receptor can comprise etanercept.

In some embodiments, the antibody that binds to TNF-alpha or a TNF-alpha binding fragment thereof comprises a VHH. In some embodiments, the TNF-alpha binding VHH comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the TNF-alpha binding VHH comprises an amino acid sequence at least about 85%, 90%, 95%, 97%, 98%, or 99% identical to that set forth in any one of SEQ ID NOs: 118 or 125.

### Dual Inhibitors of IL-23 and TNF-Alpha

Inhibitors described here can be an inhibitor of both IL-23 and TNF-alpha. In some embodiments, the inhibitor of both IL-23 and TNF-alpha can be a small molecule inhibitor. In some embodiments, the inhibitor of both IL-23 and TNF-alpha can comprise an antibody that binds IL-23 and an IL-23 binding fragment thereof. In some embodiments, the inhibitor of both IL-23 and TNF-alpha can comprise an antibody that binds TNF-alpha and a TNF-alpha binding fragment thereof. In some embodiments, the inhibitor of both IL-23 and TNF-alpha can comprise an antibody that binds IL-23, an IL-23 binding fragment thereof, an antibody that binds TNF-alpha, a TNF-alpha binding fragment thereof, or combinations thereof. In some embodiments, the inhibitor of both IL-23 and TNF-alpha can comprise a covalent fusion of an inhibitor of IL-23 and an inhibitor of TNF-alpha. In some embodiments, the inhibitor of both IL-23 and TNF-alpha can comprise a peptide linker.

In some embodiments, the inhibitor of both IL-23 and TNF-alpha comprises a combination therapeutic that is a co-formulated composition comprising both an IL-23 and a TNF-alpha inhibitor. Such combination therapeutics can be two separate small molecules. In some embodiments, the small molecule inhibitor of IL-23 comprises STA-5326, peptide 2305, or combinations thereof. In some embodiments, the small molecule inhibitor of TNF-alpha comprises SAR441566, TIM1, TIM1c, SPD-304, MYMD-1, or combinations thereof.

In some embodiments, the inhibitor of both IL-23 and TNF-alpha comprises a polypeptide that binds to both IL-23 and TNF-alpha (e.g., an IL-23 and TNF-alpha bispecific antibody). In some embodiments, the polypeptide that binds to both IL-23 and TNF-alpha comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region that binds IL-23 comprises an amino acid sequence at least about 85%, 90%, 95%, 97%, 98%, or 99% identical to that set forth in any one of SEQ ID 10-12; and the second binding region that binds TNF-alpha comprises an amino acid sequence at least about 85%, 90%, 95%, 97%, 98%, or 99% identical to that set forth in any one of SEQ ID NOs: 118 or 125. In some embodiments, the first binding region and the second binding region are coupled by a linker. In some embodiments, the first binding region and the second binding region are coupled by a protease-labile linker.

### Inhibitors of JAKs

Inhibitors described here can be a JAK inhibitor. JAK inhibitors described herein can be an inhibitor of JAK1, JAK2, JAK3, TYK2, or combinations thereof. In some embodiments, JAK inhibitors described herein can inhibit JAK/STAT signaling. In some embodiments, JAK inhibitors described herein can inhibit JAK phosphorylation. In some embodiments, JAK inhibitors described herein can inhibit JAK transphosphorylation. In some embodiments, JAK inhibitors described herein can inhibit JAK phosphorylation of receptor chains. In some embodiments, JAK inhibitors described herein can inhibit JAK phosphorylation of STAT proteins. In some embodiments, JAK inhibitors described herein can bind to JAK1, JAK2, JAK3, TYK2, or combinations thereof. In some embodiments, JAK inhibitors described herein can bind to the adenosine triphosphate (ATP) binding pocket of JAK1, JAK2, JAK3, TYK2, or combinations thereof. In some embodiments, JAK inhibitors described herein can bind to an epitope proximal to the ATP binding pocket of JAK1, JAK2, JAK3, TYK2, or combinations thereof, such that the ATP binding pocket is occluded. In some embodiments, JAK inhibitors described herein inhibits ATP binding to the ATP binding pocket of JAK1, JAK2, JAK3, TYK2, or combinations thereof. In some embodiments, JAK inhibitors described herein can inhibit JAK/STAT signaling. In some embodiments, JAK inhibitors described herein can inhibit biological pathways activated downstream of JAK/STAT signaling. In some embodiments, JAK inhibitors described herein can inhibit expression of pro-inflammatory genes.

In some embodiments, the JAK inhibitor comprises a small molecule JAK inhibitor of JAK1, JAK2, JAK3, TYK2, or combinations thereof. In some embodiments, the small molecule JAK inhibitor of JAK1, JAK2, JAK3, TYK2, or combinations thereof comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

In some embodiments, the JAK inhibitor comprises an antibody or antibody fragment that binds to JAK1, JAK2, JAK3, TYK2, or combinations thereof.

In some embodiments, the JAK inhibitor comprises a TYK2 inhibitor. In some embodiments, the TYK2 inhibitor comprises TAK-279, VTX958, ESK-001, or combinations thereof.

### S1P Modulators

Modulators of S1P described herein can bind to S1PR1, S1PR2, S1PR3, S1PR4, S1PR5, or combinations thereof. In some embodiments, modulators of S1P described herein can agonize S1PR1, S1PR2, S1PR3, S1PR4, S1PR5, or combinations thereof. In some embodiments, modulators of S1P described herein can antagonize S1PR1, S1PR2, S1PR3, S1PR4, S1PR5, or combinations thereof. In some embodiments, modulators of S1P described herein can regulate S1PR surface density. In some embodiments, modulators of S1P described herein can decrease S1PR surface density. In some embodiments, modulators of S1P described herein can agonize S1PR and induce S1PR internalization. In some embodiments, modulators of S1P described herein can inhibit lymphocyte egress from lymphoid tissue.

In some embodiments, the S1P modulator comprises a small molecule S1P modulator. In some embodiments, the small molecule S1P modulator can agonize or antagonize S1PR1, S1PR2, S1PR3, S1PR4, S1PR5, or combinations thereof. In some embodiments, the small molecule S1P modulator comprises fingolimod, ozanimod, siponimod, ponesimod, or combinations thereof.

### Inhibitors of TL1A

Inhibitors of TL1A described herein can antagonize TL1A-induced cellular processes, including pro-inflammatory pathways. In some embodiments, inhibitors of TL1A described herein can bind to the soluble form of TL1A. In some embodiments, inhibitors of TL1A described herein can bind to the membrane-bound form of TL1A. In some embodiments, inhibitors of TL1A described herein can bind to the membrane-bound trimer form of TL1A. In some embodiments, inhibitors of TL1A described herein can bind to soluble forms of TNF-alpha, monomeric forms of membrane-bound TL1A, trimeric forms of membrane-bound TL1A, or combinations thereof. In some embodiments, inhibitors of TL1A described herein can bind to DR3. In some embodiments, inhibitors of TL1A described herein can bind to decoy receptor 3. In some embodiments, inhibitors of TL1A described herein can block binding of membrane-bound monomers of TL1A to DR3. In some embodiments, inhibitors of TL1A described herein can block binding of membrane-bound monomers of TL1A to decoy receptor 3. In some embodiments, inhibitors of TL1A described herein can block binding of membrane-bound trimers of TL1A to DR3. In some embodiments, inhibitors of TL1A described herein can block binding of membrane-bound trimers of TL1A to decoy receptor 3. In some embodiments, inhibitors of TL1A described herein can block binding of soluble TL1A to DR3. In some embodiments, inhibitors of TL1A described herein can block binding of soluble TL1A to decoy receptor 3. In some embodiments, inhibitors of TL1A described herein can block binding of membrane-bound monomers of TL1A, membrane-bound trimers of TL1A, soluble TL1A, or combinations thereof, to DR3. In some embodiments, inhibitors of TL1A described herein can block binding of membrane-bound monomers of TL1A, membrane-bound trimers of TL1A, soluble TL1A, or combinations thereof, to decoy receptor 3. In some embodiments, inhibitors of TL1A described herein can block signaling pathways activated in response to TL1A. In some embodiments, inhibitors of TL1A described herein can inhibit NF-κB and/or MAPK signaling. In some embodiments, inhibitors of TL1A described herein can inhibit cellular processes downstream of NF-κB and/or MAPK signaling. In some embodiments, inhibitors of TL1A described herein can inhibit expression of pro-inflammatory genes.

In some embodiments, the inhibitor of TL1A comprises a small molecule inhibitor of TL1A. In some embodiments, the inhibitor of TL1A comprises an antibody or antibody fragment that binds to membrane-bound monomers of TL1A, membrane-bound trimers of TL1A, soluble TL1A, or combinations thereof. In some embodiments, the inhibitor of TL1A comprises PRA023 (also referred to as Tulisokibart), PF-06480605 (also referred to as RVT-3101), or combinations thereof.

### Improvements in Clinical Outcomes

The treatments prescribed herein can result in improvement in outcomes for patients afflicted with autoimmune and/or inflammatory conditions. In some embodiments, the treatments prescribed herein can improve symptoms of patients afflicted with autoimmune and/or inflammatory conditions. In some embodiments, the treatments prescribed herein can provide short-term relief of patients afflicted with autoimmune and/or inflammatory conditions. In some embodiments, the treatments prescribed herein can provide long-term relief of patients afflicted with autoimmune and/or inflammatory conditions.

In some embodiments, the treatments prescribed herein can reduce the severity of diarrhea in patients afflicted with autoimmune and/or inflammatory conditions. In some embodiments, the treatments prescribed herein can reduce the severity of urgency in patients afflicted with autoimmune and/or inflammatory conditions. In some embodiments, the treatments prescribed herein can improve stool consistency in patients afflicted with autoimmune and/or inflammatory conditions. In some embodiments, the treatments prescribed herein can reduce the frequency of diarrhea in patients afflicted with autoimmune and/or inflammatory conditions. In some embodiments, the treatments prescribed herein can reduce the severity, the stool consistency, the frequency, or combinations thereof, of diarrhea in patients afflicted with autoimmune and/or inflammatory conditions.

In some embodiments, the treatments prescribed herein can reduce the severity of perianal conditions, such as skin tags, anal fissures, and perianal fistulae, in patients afflicted with autoimmune and/or inflammatory conditions. In some embodiments, the treatments prescribed herein can reduce the severity of rectal bleeding in patients afflicted with autoimmune and/or inflammatory conditions. In some embodiments, the treatments prescribed herein can reduce the frequency of rectal bleeding in patients afflicted with autoimmune and/or inflammatory conditions. In some embodiments, the treatments prescribed herein can reduce the severity and/or frequency of rectal bleeding in patients afflicted with autoimmune and/or inflammatory conditions. In some embodiments, the treatments prescribed herein can reduce the amount of blood and/or mucus found in the stool of patients afflicted with autoimmune and/or inflammatory conditions.

In some embodiments, the treatments prescribed herein can reduce the severity of abdominal cramping in patients afflicted with autoimmune and/or inflammatory conditions. In some embodiments, the treatments prescribed herein can reduce the frequency of abdominal cramping in patients afflicted with autoimmune and/or inflammatory conditions. In some embodiments, the treatments prescribed herein can reduce the severity and/or frequency of abdominal cramping in patients afflicted with autoimmune and/or inflammatory conditions.

In some embodiments, the treatments prescribed herein can reduce the severity of abdominal pain in patients afflicted with autoimmune and/or inflammatory conditions. In some embodiments, the treatments prescribed herein can reduce the frequency of abdominal pain in patients afflicted with autoimmune and/or inflammatory conditions. In some embodiments, the treatments prescribed herein can reduce the severity and/or frequency of abdominal pain in patients afflicted with autoimmune and/or inflammatory conditions.

In some embodiments, the treatments prescribed herein can improve clinical outcomes as measured by hematological parameters in patients afflicted by autoimmune and/or inflammatory conditions. A non-limiting list of hematological parameters include white blood cell count, hemoglobin and hematocrit count, platelet count, iron saturation, erythrocyte sedimentation rate, and C-reactive protein concentration. In some embodiments, the treatments prescribed herein can regulate fecal calprotectin concentration in the stool of patients afflicted by autoimmune and/or inflammatory conditions.

In some embodiments, the treatments prescribed herein can improve clinical outcomes as measured by serological markers in patients afflicted by autoimmune and/or inflammatory conditions. A non-limiting list of serological parameters include anti-neutrophil cytoplasm antibody concentration and anti-Saccharomyces cerevisiae antibody concentration.

In some embodiments, the treatments prescribed herein can improve clinical outcomes as determined by endoscopic sampling or sampling by surgical resection. In some embodiments, the treatments prescribed herein can improve clinical outcomes as determined by gross features. In some embodiments, the treatments prescribed herein can improve clinical outcomes as determined by gross features, wherein the gross feature observed is the normalization of GI histology, such as normalization of mucosal architecture, normalization of superficial epithelial features, normalization of epitheliallar components, and normalization of lamina propia cellularity. In some embodiments, the treatments prescribed herein can improve clinical outcomes as determined by gross features, wherein the gross feature observed is a decrease in the area afflicted by GI inflammation. In some embodiments, the treatments prescribed herein can improve clinical outcomes as determined by gross features, wherein the gross feature observed is a decrease in the severity of GI inflammation. In some embodiments, the treatments prescribed herein can improve clinical outcomes as determined by gross features, wherein the gross feature observed is a decrease in mucosal alterations, a decrease in pseudopoylp count, a decrease in erosions, a decrease in ulcerations, a decrease in fistulae, or combinations thereof. In some embodiments, the treatments prescribed herein can improve clinical outcomes as determined by gross features, wherein the gross feature observed is an increase in mucosal wall thickness. In some embodiments, the treatments prescribed herein can improve clinical outcomes as determined by gross features, wherein the gross feature observed is a reduction in size and/or count of lesions.

In some embodiments, the treatments prescribed herein can ameliorate or prevent extraintestinal manifestations (EIMs). A non-limiting list of EIMs includes arthropathy, arthritis, ankylosing spondylitis, uveitis, scleritis, episcleritis, primary scleorosing cholangitis, metabolic bone disease, and pulmonary or deep-venous thromboembolic disease.

In some embodiments, the treatments prescribed herein can ameliorate or prevent cutaneous manifestations of IBDs. A non-limiting list of cutaneous manifestations of includes erythema nodosum, pyoderma gangrenosum, Sweet's syndrome, and oral lesions.

In some embodiments, the treatments prescribed herein can reduce the severity of fatigue experienced by patients afflicted with autoimmune and/or inflammatory conditions. In some embodiments, the treatments prescribed herein can reduce the frequency of fatigue experienced by patients afflicted with autoimmune and/or inflammatory conditions. In some embodiments, the treatments prescribed herein can reduce the severity and/or frequency of fatigue experienced by patients afflicted with autoimmune and/or inflammatory conditions.

In some embodiments, the treatments prescribed herein can reduce the severity of fevers in patients afflicted with autoimmune and/or inflammatory conditions. In some embodiments, the treatments prescribed herein can reduce the frequency of fevers in patients afflicted with autoimmune and/or inflammatory conditions. In some embodiments, the treatments prescribed herein can reduce the severity and/or frequency of fevers in patients afflicted with autoimmune and/or inflammatory conditions.

In some embodiments, the treatments prescribed herein can ameliorate or prevent unintended weight loss in patients afflicted with autoimmune and/or inflammatory conditions. In some embodiments, the treatments prescribed herein can ameliorate or prevent pain experienced by patients afflicted with autoimmune and/or inflammatory conditions. In some embodiments, the treatments prescribed herein can improve appetite in patients afflicted with autoimmune and/or inflammatory conditions. In some embodiments, the treatments prescribed herein can improve sleep quality in patients afflicted with autoimmune and/or inflammatory conditions. In some embodiments, the treatments prescribed herein can improve the quality of life in patients afflicted with autoimmune and/or inflammatory conditions.

### Therapeutic Methods

Described herein are methods of treating an autoimmune or inflammatory condition in an individual in need thereof. In some embodiments, the autoimmune or inflammatory condition is an autoimmune or inflammatory condition of the GI tract. In some embodiments, the autoimmune or inflammatory condition of the GI tract comprises IBD. In some embodiments, the autoimmune or inflammatory condition of the GI tract comprises Crohn's disease, ulcerative colitis, colitis, microscopic colitis, celiac disease, mucositis, pouchitis, or gastritis. In some embodiments, the autoimmune or inflammatory condition of the GI tract comprises GI cancers. In some embodiments, the GI cancers comprise anal cancer, bile duct cancer, colon cancer, esophageal cancer, small intestine cancer, and gastric cancer. Treatment regimen parameters include, but are not limited to, the administration route, the treatment schedule, and the therapeutic dosage.

### Administration Route

The methods of treatment described herein can encompass systemic administration. The methods of treatment described herein can encompass local administration. In certain embodiments the local administration is oral administration of a polypeptide, immunoglobulin, or VHH that does not achieve systemic availability by an oral route. The methods of treatment described herein can be administered to an individual in need thereof through parenteral administration or enteral administration. The enteral administration route encompasses administration involving the GI tract. In some embodiments, enteral administration encompasses administration to the alimentary canal. Non-limiting examples of enteral administration include oral administration, rectal administration, or direct administration to the alimentary canal or a portion thereof. In some embodiments, enteral administration can be provided as a suppository. In some embodiments, enteral administration is oral administration. In some embodiments, enteral administration is administered by gavage or intubation. In some embodiments, enteral administration can be provided orally as a liquid, as a nutraceutical, or as a powder. In some embodiments, enteral administration can be provided orally in the form of a pill. In some embodiments, enteral administration can be provided orally to a specified area of G.I. tract in the form of a capsule. In some embodiments, enteral administration can be provided orally to the colon in the form of a capsule. In contrast, the parenteral administration route encompasses any administration route that is not enteral. In some instances, parenteral administration can be achieved through needle injection or administration through an implanted catheter. Non-limiting examples of parenteral administration include intramuscular, subcutaneous, intravenous, intradermal, intrathecal, nasal, intraocular, and inhalation administration. In some embodiments, the parenteral administration route is subcutaneous administration. In some embodiments, the parenteral administration route is intravenous administration. In some embodiments, parenteral administration can enable systemic delivery of the therapeutic. In some embodiments, parenteral administration can enable local delivery of the therapeutic. In some embodiments, enteral administration can enable systemic delivery of the therapeutic. In some embodiments, enteral administration can enable local delivery of the therapeutic.

### Treatment Schedule

In some embodiments, the methods of treatment described herein can be comprise a first treatment regimen and a second treatment regimen. In some embodiments, the first treatment regimen can be systemic administration. In some embodiments, the first treatment regimen can be local administration. In some embodiments, the second treatment regimen can be systemic administration. In some embodiments, the second treatment regimen can be local administration. In some embodiments, the start of the first treatment regimen and the start of the second treatment regimen can start at the same time. In some embodiments, the start of the first treatment regimen and the start of the second treatment regimen can be temporally distinct. In some embodiments, the end of the first treatment regime and the end of the second regime can occur at the same time. In some embodiments, the end of the first treatment regime and the end of the second regime can be temporally distinct. In some embodiments, the methods of treatment described herein can include administration of a first therapeutic, wherein the first therapeutic can be a first inhibitor, and a second therapeutic, wherein the second therapeutic can be a second inhibitor.

In some embodiments, the first treatment regimen can be systemic administration and the second treatment regimen can be local administration. In some embodiments, the start of the first systemic treatment regimen and the start of the second local treatment regimen can occur at the same time. In some embodiments, the start of the second local treatment regimen can occur after administration of the first systemic administration. In some embodiments, the end of the first systemic treatment regimen and the end of the second systemic regimen can end at the same time. In some embodiments, the end of the first systemic treatment regimen can end before the end of the second systemic treatment regimen. In some embodiments, the first systemic treatment regimen can end 1 week, 2 weeks, 3 weeks, 4 weeks, 2 months, 3 months, or 4 months after the start of the first systemic treatment. In some embodiments, the first systemic treatment regimen can end 1 week, 2 weeks, 3 weeks, 4 weeks, 2 months, 3 months, 4 months, or 12 months after the start of the first systemic treatment. In some embodiments, the first systemic treatment regimen can end after any of the improvements in clinical outcomes described herein are observed while the second local treatment regimen continues. In some embodiments, the second local treatment regimen can start after any of the improvements in clinical outcomes described herein are observed in response to the first systemic treatment regimen. In some embodiments, the second local treatment regimen can end 1 week, 2 weeks, 3 weeks, 4 weeks, 2 months, 3 months, or 4 months after the start of the first systemic treatment. In some embodiments, the second local treatment regimen can end 1 week, 2 weeks, 3 weeks, 4 weeks, 2 months, 3 months, 4 months, or 12 months after the start of the first systemic treatment. In some embodiments, the second local treatment can end after any of the improvements in clinical outcomes described herein are observed.

In some embodiments, administration of a first therapeutic can comprise administration of an inhibitor of IL-23. In some embodiments, administration of a first therapeutic can comprise administration of an inhibitor of TNF-alpha. In some embodiments, administration of a first therapeutic can comprise administration of an inhibitor of both IL-23 and TNF-alpha. In some embodiments, administration of a first therapeutic can comprise administration of a dual inhibitor of both IL-23 and TNF-alpha. In some embodiments, administration of a first therapeutic can comprise administration of a combination therapeutic comprising an inhibitor of IL-23 and an inhibitor of TNF-alpha. In some embodiments, an inhibitor of IL-23 and an inhibitor of TNF-alpha can be administered separately.

In some embodiments, administration of a first therapeutic can comprise administration of a JAK inhibitor. In some embodiments, administration of a first therapeutic can comprise administration of an inhibitor of JAK1, JAK2, JAK3, TYK2, or combinations thereof. In some embodiments, administration of a first therapeutic can comprise administration of a S1P modulator. In some embodiments, administration of a first therapeutic can comprise an inhibitor of TL1A. In some embodiments, administration of a first therapeutic can comprise a JAK inhibitor, a S1P modulator, an inhibitor of TL1A, or combinations thereof.

In some embodiments, administration of a second therapeutic can comprise administration of an inhibitor of IL-23. In some embodiments, administration of a second therapeutic can comprise administration of an inhibitor of TNF-alpha. In some embodiments, administration of a second therapeutic can be administration of an inhibitor of both IL-23 and TNF-alpha. In some embodiments, administration of a second therapeutic can comprise administration of a dual inhibitor of both IL-23 and TNF-alpha. In some embodiments, administration of the second therapeutic can comprise administration of a combination therapeutic comprising an inhibitor of IL-23 and an inhibitor of TNF-alpha. In some embodiments, an inhibitor of IL-23 and an inhibitor of TNF-alpha can be administered separately.

In some embodiments, administration of a second therapeutic can comprise administration of a JAK inhibitor. In some embodiments, administration of a second therapeutic can comprise administration of an inhibitor of JAK1, JAK2, JAK3, TYK2, or combinations thereof. In some embodiments, administration of a second therapeutic can comprise administration of a S1P modulator. In some embodiments, administration of a second therapeutic can comprise an inhibitor of TL1A. In some embodiments, administration of a second therapeutic can comprise a JAK inhibitor, a S1P modulator, an inhibitor of TL1A, or combinations thereof.

The first treatment regimen can encompass parenteral administration of a therapeutic, enteral administration of a therapeutic, or combinations thereof. The second treatment regimen can encompass parenteral administration of a therapeutic, enteral administration of a therapeutic, or combinations thereof. Administration of a first therapeutic can be parenteral, enteral, or combinations thereof. Administration of a second therapeutic can be parenteral, enteral, or combinations thereof. In some embodiments, administration of the first therapeutic is parenteral and administration of the second therapeutic is parenteral. In some embodiments, administration of the first therapeutic is parenteral and administration of the second therapeutic is enteral. In some embodiments, administration of the first therapeutic is enteral and administration of the second therapeutic is enteral. In some embodiments, administration of the first therapeutic is enteral and administration of the second therapeutic is parenteral.

In some embodiments, administration of the first therapeutic can occur prior to administration of the second therapeutic. In some embodiments, administration of the first therapeutic can occur prior to administration of the second therapeutic by at least 24 hours. In some embodiments, administration of the first therapeutic can occur prior to administration of the second therapeutic by at least 2 days. In some embodiments, administration of the first therapeutic can occur prior to administration of the second therapeutic by at least 3 days. In some embodiments, administration of the first therapeutic can occur prior to administration of the second therapeutic by at least 5 days. In some embodiments, administration of the first therapeutic can occur prior to administration of the second therapeutic by at least 7 days. In some embodiments, administration of the first therapeutic can occur prior to administration of the second therapeutic by at least 14 days. In some embodiments, administration of the first therapeutic can occur prior to administration of the second therapeutic by at least one month. In some embodiments, administration of the first therapeutic can occur prior to administration of the second therapeutic by at least two months. In some embodiments, administration of the first therapeutic can occur prior to administration of the second therapeutic by at least three months. In some embodiments, administration of the first therapeutic can occur prior to administration of the second therapeutic by at least four months.

In some embodiments, administration of the second therapeutic can occur after administration of the first therapeutic. In some embodiments, administration of the second therapeutic can occur after administration of the first therapeutic by at least 24 hours. In some embodiments, administration of the second therapeutic can occur after administration of the first therapeutic by at least 2 days. In some embodiments, administration of the second therapeutic can occur after administration of the first therapeutic by at least 3 days. In some embodiments, administration of the second therapeutic can occur after administration of the first therapeutic by at least 5 days. In some embodiments, administration of the second therapeutic can occur after administration of the first therapeutic by at least 7 days. In some embodiments, administration of the second therapeutic can occur after administration of the first therapeutic by at least 14 days. In some embodiments, administration of the second therapeutic can occur after administration of the first therapeutic by at least one month. In some embodiments, administration of the second therapeutic can occur after administration of the first therapeutic by at least two months. In some embodiments, administration of the second therapeutic can occur after administration of the first therapeutic by at least three months. In some embodiments, administration of the second therapeutic can occur after administration of the first therapeutic by at least four months.

In some embodiments, the methods of treatment described herein can further include administration of a third therapeutic, wherein the third therapeutic can be a third inhibitor. In some embodiments, there can be a temporal delay between administration of the first therapeutic, administration of the second therapeutic, and administration of the third therapeutic. In some embodiments, administration of the first therapeutic, administration of the second therapeutic, and administration of the third therapeutic can occur at the same time.

In some embodiments, administration of a third therapeutic can comprise administration of a JAK inhibitor. In some embodiments, administration of a third therapeutic can comprise administration of an inhibitor of JAK1, JAK2, JAK3, TYK2, or combinations thereof. In some embodiments, administration of a third inhibitor can comprise administration of a S1P modulator. In some embodiments, administration of a third inhibitor can comprise an inhibitor of TL1A. In some embodiments, administration of a third therapeutic can comprise a JAK inhibitor, a S1P modulator, an inhibitor of TL1A, or combinations thereof.

In some embodiments, administration of the third therapeutic can occur prior to administration of the second therapeutic. In some embodiments, administration of the third therapeutic can occur simultaneously to administration of the second therapeutic. In some embodiments, administration of the third therapeutic occurs prior to administration of the second therapeutic. In some embodiments, administration of the third therapeutic occurs prior to administration of the second therapeutic by at least 24 hours. In some embodiments, administration of the third therapeutic occurs prior to administration of the second therapeutic by at least 2 days. In some embodiments, administration of the third therapeutic occurs prior to administration of the second therapeutic by at least 3 days. In some embodiments, administration of the third therapeutic occurs prior to administration of the second therapeutic by at least 5 days. In some embodiments, administration of the third therapeutic occurs prior to administration of the second therapeutic by at least 7 days. In some embodiments, administration of the third therapeutic occurs prior to administration of the second therapeutic by at least 14 days. In some embodiments, administration of the third therapeutic occurs prior to administration of the second therapeutic by at least one month. In some embodiments, administration of the third therapeutic occurs prior to administration of the second therapeutic by at least two months. In some embodiments, administration of the third therapeutic occurs prior to administration of the second therapeutic by at least three months. In some embodiments, administration of the third therapeutic occurs prior to administration of the second therapeutic by at least four months.

In some embodiments, administration of the third therapeutic can occur after administration of the second therapeutic. In some embodiments, administration of the third therapeutic occurs after administration of the second therapeutic. In some embodiments, administration of the third therapeutic occurs after administration of the second therapeutic by at least 24 hours. In some embodiments, administration of the third therapeutic occurs after administration of the second therapeutic by at least 2 days. In some embodiments, administration of the third therapeutic occurs after administration of the second therapeutic by at least 3 days. In some embodiments, administration of the third therapeutic occurs after administration of the second therapeutic by at least 5 days. In some embodiments, administration of the third therapeutic occurs after administration of the second therapeutic by at least 7 days. In some embodiments, administration of the third therapeutic occurs after administration of the second therapeutic by at least 14 days. In some embodiments, administration of the third therapeutic occurs after administration of the second therapeutic by at least one month. In some embodiments, administration of the third therapeutic occurs after administration of the second therapeutic by at least two months. In some embodiments, administration of the third therapeutic occurs after administration of the second therapeutic by at least three months. In some embodiments, administration of the third therapeutic occurs after administration of the second therapeutic by at least four months.

In some embodiments, administration of the third therapeutic can occur after administration of the first therapeutic. In some embodiments, administration of the third therapeutic occurs after administration of the first therapeutic. In some embodiments, administration of the third therapeutic occurs after administration of the first therapeutic by at least 24 hours. In some embodiments, administration of the third therapeutic occurs after administration of the first therapeutic by at least 2 days. In some embodiments, administration of the third therapeutic occurs after administration of the first therapeutic by at least 3 days. In some embodiments, administration of the third therapeutic occurs after administration of the first therapeutic by at least 5 days. In some embodiments, administration of the third therapeutic occurs after administration of the first therapeutic by at least 7 days. In some embodiments, administration of the third therapeutic occurs after administration of the first therapeutic by at least 14 days. In some embodiments, administration of the third therapeutic occurs after administration of the first therapeutic by at least one month. In some embodiments, administration of the third therapeutic occurs after administration of the first therapeutic by at least two months. In some embodiments, administration of the third therapeutic occurs after administration of the first therapeutic by at least three months. In some embodiments, administration of the third therapeutic occurs after administration of the first therapeutic by at least four months.

The methods of treatment described herein can comprise at least two treatment regimens, wherein each treatment regimen includes at least one instance of administration of the therapeutic. In some embodiments, each treatment regimen includes at least two instances of administration of the therapeutic. In some embodiments, each treatment regimen includes at least four instances of administration of the therapeutic. In some embodiments, each treatment regimen includes multiple instances of administration of the therapeutic.

Each instance of administration of the therapeutic can be spaced apart with time intervals. Each instance of administration of the therapeutic can be spaced apart with recurring fixed time intervals. Each instance of administration of the therapeutic can be spaced apart with recurring variable time intervals. In some embodiments, each instance of administration of the therapeutic can be spaced apart by at least 24 hours. In some embodiments, each instance of administration of the therapeutic can be spaced apart by at least 2 days. In some embodiments, each instance of administration of the therapeutic can be spaced apart by at least 3 days. In some embodiments, each instance of administration of the therapeutic can be spaced apart by at least 5 days. In some embodiments, each instance of administration of the therapeutic can be spaced apart by at least 7 days. In some embodiments, each instance of administration of the therapeutic can be spaced apart by at least 14 days.

In some embodiments, each instance of administration of the therapeutic can be spaced apart by at least a 24-hour interval, wherein administration of the therapeutic recurs for at least 1 week. In some embodiments, each instance of administration of the therapeutic can be spaced apart by at least a 24-hour interval, wherein administration of the therapeutic recurs for at least 2 weeks. In some embodiments, each instance of administration of the therapeutic can be spaced apart by at least a 24-hour interval, wherein administration of the therapeutic recurs for at least 3 weeks. In some embodiments, each instance of administration of the therapeutic can be spaced apart by at least a 24-hour interval, wherein administration of the therapeutic recurs for at least 4 weeks. In some embodiments, each instance of administration of the therapeutic can be spaced apart by at least a 24-hour interval, wherein administration of the therapeutic recurs for at least 8 weeks. In some embodiments, each instance of administration of the therapeutic can be spaced apart by at least a 24-hour interval, wherein administration of the therapeutic recurs for at least 12 weeks. In some embodiments, each instance of administration of the therapeutic can be spaced apart by at least a 24-hour interval, wherein administration of the therapeutic recurs for at least 16 weeks. In some embodiments, each instance of administration of the therapeutic can be spaced apart by at least a 24-hour interval, wherein administration of the therapeutic recurs for at least 24 weeks. In some embodiments, each instance of administration of the therapeutic can be spaced apart by at least a 24-hour interval, wherein administration of the therapeutic recurs for at least 48 weeks. In some embodiments, each instance of administration of the therapeutic can be spaced apart by at least a 24-hour interval, wherein administration of the therapeutic recurs for at least 52 weeks. In some embodiments, each instance of administration of the therapeutic can be spaced apart by at least a 24-hour interval, wherein administration of the therapeutic recurs for at least 56 weeks. In some embodiments, each instance of administration of the therapeutic can be spaced apart by at least a 24-hour interval, wherein administration of the therapeutic recurs for at least 112 weeks.

In some embodiments, each instance of administration of the therapeutic can be spaced apart by at least 2 days, wherein administration of the therapeutic recurs for at least 1 week. In some embodiments, each instance of administration of the therapeutic can be spaced apart by at least at least 2 days, wherein administration of the therapeutic recurs for at least 2 weeks. In some embodiments, each instance of administration of the therapeutic can be spaced apart by at least at least 2 days, wherein administration of the therapeutic recurs for at least 3 weeks. In some embodiments, each instance of administration of the therapeutic can be spaced apart by at least at least 2 days, wherein administration of the therapeutic recurs for at least 4 weeks. In some embodiments, each instance of administration of the therapeutic can be spaced apart by at least at least 2 days, wherein administration of the therapeutic recurs for at least 8 weeks. In some embodiments, each instance of administration of the therapeutic can be spaced apart by at least at least 2 days, wherein administration of the therapeutic recurs for at least 12 weeks. In some embodiments, each instance of administration of the therapeutic can be spaced apart by at least at least 2 days, wherein administration of the therapeutic recurs for at least 16 weeks. In some embodiments, each instance of administration of the therapeutic can be spaced apart by at least at least 2 days, wherein administration of the therapeutic recurs for at least 24 weeks. In some embodiments, each instance of administration of the therapeutic can be spaced apart by at least at least 2 days, wherein administration of the therapeutic recurs for at least 48 weeks. In some embodiments, each instance of administration of the therapeutic can be spaced apart by at least at least 2 days, wherein administration of the therapeutic recurs for at least 52 weeks. In some embodiments, each instance of administration of the therapeutic can be spaced apart by at least at least 2 days, wherein administration of the therapeutic recurs for at least 56 weeks. In some embodiments, each instance of administration of the therapeutic can be spaced apart by at least at least 2 days, wherein administration of the therapeutic recurs for at least 112 weeks.

In some embodiments, each instance of administration of the therapeutic can be spaced apart by at least 3 days, wherein administration of the therapeutic recurs for at least 1 week. In some embodiments, each instance of administration of the therapeutic can be spaced apart by at least at least 3 days, wherein administration of the therapeutic recurs for at least 2 weeks. In some embodiments, each instance of administration of the therapeutic can be spaced apart by at least at least 3 days, wherein administration of the therapeutic recurs for at least 3 weeks. In some embodiments, each instance of administration of the therapeutic can be spaced apart by at least at least 3 days, wherein administration of the therapeutic recurs for at least 4 weeks. In some embodiments, each instance of administration of the therapeutic can be spaced apart by at least at least 3 days, wherein administration of the therapeutic recurs for at least 8 weeks. In some embodiments, each instance of administration of the therapeutic can be spaced apart by at least at least 3 days, wherein administration of the therapeutic recurs for at least 12 weeks. In some embodiments, each instance of administration of the therapeutic can be spaced apart by at least at least 3 days, wherein administration of the therapeutic recurs for at least 16 weeks. In some embodiments, each instance of administration of the therapeutic can be spaced apart by at least at least 3 days, wherein administration of the therapeutic recurs for at least 24 weeks. In some embodiments, each instance of administration of the therapeutic can be spaced apart by at least at least 3 days, wherein administration of the therapeutic recurs for at least 48 weeks. In some embodiments, each instance of administration of the therapeutic can be spaced apart by at least at least 3 days, wherein administration of the therapeutic recurs for at least 52 weeks. In some embodiments, each instance of administration of the therapeutic can be spaced apart by at least at least 3 days, wherein administration of the therapeutic recurs for at least 56 weeks. In some embodiments, each instance of administration of the therapeutic can be spaced apart by at least at least 3 days, wherein administration of the therapeutic recurs for at least 112 weeks.

In some embodiments, each instance of administration of the therapeutic can be spaced apart by at least 5 days, wherein administration of the therapeutic recurs for at least 1 week. In some embodiments, each instance of administration of the therapeutic can be spaced apart by at least at least 5 days, wherein administration of the therapeutic recurs for at least 2 weeks. In some embodiments, each instance of administration of the therapeutic can be spaced apart by at least at least 5 days, wherein administration of the therapeutic recurs for at least 3 weeks. In some embodiments, each instance of administration of the therapeutic can be spaced apart by at least at least 5 days, wherein administration of the therapeutic recurs for at least 4 weeks. In some embodiments, each instance of administration of the therapeutic can be spaced apart by at least at least 5 days, wherein administration of the therapeutic recurs for at least 8 weeks. In some embodiments, each instance of administration of the therapeutic can be spaced apart by at least at least 5 days, wherein administration of the therapeutic recurs for at least 12 weeks. In some embodiments, each instance of administration of the therapeutic can be spaced apart by at least at least 5 days, wherein administration of the therapeutic recurs for at least 16 weeks. In some embodiments, each instance of administration of the therapeutic can be spaced apart by at least at least 5 days, wherein administration of the therapeutic recurs for at least 24 weeks. In some embodiments, each instance of administration of the therapeutic can be spaced apart by at least at least 5 days, wherein administration of the therapeutic recurs for at least 48 weeks. In some embodiments, each instance of administration of the therapeutic can be spaced apart by at least at least 5 days, wherein administration of the therapeutic recurs for at least 52 weeks. In some embodiments, each instance of administration of the therapeutic can be spaced apart by at least at least 5 days, wherein administration of the therapeutic recurs for at least 56 weeks. In some embodiments, each instance of administration of the therapeutic can be spaced apart by at least at least 5 days, wherein administration of the therapeutic recurs for at least 112 weeks.

In some embodiments, each instance of administration of the therapeutic can be spaced apart by at least 7 days, wherein administration of the therapeutic recurs for at least 1 week. In some embodiments, each instance of administration of the therapeutic can be spaced apart by at least at least 7 days, wherein administration of the therapeutic recurs for at least 2 weeks. In some embodiments, each instance of administration of the therapeutic can be spaced apart by at least at least 7 days, wherein administration of the therapeutic recurs for at least 3 weeks. In some embodiments, each instance of administration of the therapeutic can be spaced apart by at least at least 7 days, wherein administration of the therapeutic recurs for at least 4 weeks. In some embodiments, each instance of administration of the therapeutic can be spaced apart by at least at least 7 days, wherein administration of the therapeutic recurs for at least 8 weeks. In some embodiments, each instance of administration of the therapeutic can be spaced apart by at least at least 7 days, wherein administration of the therapeutic recurs for at least 12 weeks. In some embodiments, each instance of administration of the therapeutic can be spaced apart by at least at least 7 days, wherein administration of the therapeutic recurs for at least 16 weeks. In some embodiments, each instance of administration of the therapeutic can be spaced apart by at least at least 7 days, wherein administration of the therapeutic recurs for at least 24 weeks. In some embodiments, each instance of administration of the therapeutic can be spaced apart by at least at least 7 days, wherein administration of the therapeutic recurs for at least 48 weeks. In some embodiments, each instance of administration of the therapeutic can be spaced apart by at least at least 7 days, wherein administration of the therapeutic recurs for at least 52 weeks. In some embodiments, each instance of administration of the therapeutic can be spaced apart by at least at least 7 days, wherein administration of the therapeutic recurs for at least 56 weeks. In some embodiments, each instance of administration of the therapeutic can be spaced apart by at least at least 7 days, wherein administration of the therapeutic recurs for at least 112 weeks.

In some embodiments, each instance of administration of the therapeutic can be spaced apart by at least 14 days, wherein administration of the therapeutic recurs for at least 1 week. In some embodiments, each instance of administration of the therapeutic can be spaced apart by at least at least 14 days, wherein administration of the therapeutic recurs for at least 2 weeks. In some embodiments, each instance of administration of the therapeutic can be spaced apart by at least at least 14 days, wherein administration of the therapeutic recurs for at least 3 weeks. In some embodiments, each instance of administration of the therapeutic can be spaced apart by at least at least 14 days, wherein administration of the therapeutic recurs for at least 4 weeks. In some embodiments, each instance of administration of the therapeutic can be spaced apart by at least at least 14 days, wherein administration of the therapeutic recurs for at least 8 weeks. In some embodiments, each instance of administration of the therapeutic can be spaced apart by at least at least 14 days, wherein administration of the therapeutic recurs for at least 12 weeks. In some embodiments, each instance of administration of the therapeutic can be spaced apart by at least at least 14 days, wherein administration of the therapeutic recurs for at least 16 weeks. In some embodiments, each instance of administration of the therapeutic can be spaced apart by at least at least 14 days, wherein administration of the therapeutic recurs for at least 24 weeks. In some embodiments, each instance of administration of the therapeutic can be spaced apart by at least at least 14 days, wherein administration of the therapeutic recurs for at least 48 weeks. In some embodiments, each instance of administration of the therapeutic can be spaced apart by at least at least 14 days, wherein administration of the therapeutic recurs for at least 52 weeks. In some embodiments, each instance of administration of the therapeutic can be spaced apart by at least at least 14 days, wherein administration of the therapeutic recurs for at least 56 weeks. In some embodiments, each instance of administration of the therapeutic can be spaced apart by at least at least 14 days, wherein administration of the therapeutic recurs for at least 112 weeks.

### Therapeutic Dosage

Therapeutic dosage can impact the efficacy and durability in disease treatment. The therapeutic dosage can be defined through several metrics. In some instances, therapeutic dosage can by mass (e.g., mg). In some instances, therapeutic dosage can be defined by mass normalized to the weight of an individual in need thereof (e.g., mg/kg). In some instances, therapeutic dosage can be defined by mass normalized to the height of an individual in need thereof (e.g., mg/m). In some instances, therapeutic dosage can be defined by mass normalized to the body surface area of an individual in need thereof (e.g., mg/m²).

In some embodiments, the therapeutic can be an inhibitor of IL-23, an inhibitor of TNF-alpha, or an inhibitor of both IL-23 and TNF-alpha. In some embodiments, the inhibitor of IL-23 can be administered at a dosage of at least 15 mg per individual in need thereof. In some embodiments, the inhibitor of IL-23 can be administered at a dosage of at least 50 mg per individual in need thereof. In some embodiments, the inhibitor of IL-23 can be administered at a dosage of at least 100 mg per individual in need thereof. In some embodiments, the inhibitor of IL-23 can be administered at a dosage of at least 125 mg per individual in need thereof. In some embodiments, the inhibitor of IL-23 can be administered at a dosage of at least 150 mg per individual in need thereof. In some embodiments, the inhibitor of IL-23 can be administered at a dosage of at least 175 mg per individual in need thereof. In some embodiments, the inhibitor of IL-23 can be administered at a dosage of at least 200 mg per individual in need thereof. In some embodiments, the inhibitor of IL-23 can be administered at a dosage of at least 250 mg per individual in need thereof. In some embodiments, the inhibitor of IL-23 can be administered at a dosage of at least 300 mg per individual in need thereof. In some embodiments, the inhibitor of IL-23 can be administered at a dosage of at least 600 mg per individual in need thereof. In some embodiments, the inhibitor of IL-23 can be administered at a dosage of at least 900 mg per individual in need thereof. In some embodiments, the inhibitor of IL-23 can be administered at a dosage of at least 1500 mg per individual in need thereof.

In some embodiments, the inhibitor of IL-23 can be administered at a dosage of at least about 15 mg to about 1,500 mg. In some embodiments, the inhibitor of IL-23 can be administered at a dosage of at least about 15 mg to about 50 mg, about 15 mg to about 100 mg, about 15 mg to about 125 mg, about 15 mg to about 150 mg, about 15 mg to about 175 mg, about 15 mg to about 200 mg, about 15 mg to about 250 mg, about 15 mg to about 300 mg, about 15 mg to about 600 mg, about 15 mg to about 900 mg, about 15 mg to about 1,500 mg, about 50 mg to about 100 mg about 50 mg to about 125 mg, about 50 mg to about 150 mg, about 50 mg to about 175 mg, about 50 mg to about 200 mg, about 50 mg to about 250 mg, about 50 mg to about 300 mg, about 50 mg to about 600 mg, about 50 mg to about 900 mg, about 50 mg to about 1,500 mg, about 100 mg to about 125 mg, about 100 mg to about 150 mg, about 100 mg to about 175 mg, about 100 mg to about 200 mg, about 100 mg to about 250 mg, about 100 mg to about 300 mg, about 100 mg to about 600 mg, about 100 mg to about 900 mg, about 100 mg to about 1,500 mg, about 125 mg to about 150 mg, about 125 mg to about 175 mg, about 125 mg to about 200 mg, about 125 mg to about 250 mg, about 125 mg to about 300 mg, about 125 mg to about 600 mg, about 125 mg to about 900 mg, about 125 mg to about 1,500 mg, about 150 mg to about 175 mg, about 150 mg to about 200 mg, about 150 mg to about 250 mg, about 150 mg to about 300 mg, about 150 mg to about 600 mg, about 150 mg to about 900 mg, about 150 mg to about 1,500 mg, about 175 mg to about 200 mg, about 175 mg to about 250 mg, about 175 mg to about 300 mg, about 175 mg to about 600 mg, about 175 mg to about 900 mg, about 175 mg to about 1,500 mg, about 200 mg to about 250 mg, about 200 mg to about 300 mg, about 200 mg to about 600 mg, about 200 mg to about 900 mg, about 200 mg to about 1,500 mg, about 250 mg to about 300 mg, about 250 mg to about 600 mg, about 250 mg to about 900 mg, about 250 mg to about 1,500 mg, about 300 mg to about 600 mg, about 300 mg to about 900 mg, about 300 mg to about 1,500 mg, about 600 mg to about 900 mg, about 600 mg to about 1,500 mg, or about 900 mg to about 1,500 mg. In some embodiments, the inhibitor of IL-23 can be administered at a dosage of at least about 15 mg, about 50 mg, about 100 mg, about 125 mg, about 150 mg, about 175 mg, about 200 mg, about 250 mg about 300 mg, about 600 mg, about 900 mg, or about 1,500 mg. In some embodiments, the inhibitor of IL-23 can be administered at a dosage of at least at least about 15 mg, about 50 mg, about 100 mg, about 125 mg, about 150 mg, about 175 mg, about 200 mg, about 250 mg, about 300 mg, about 600 mg, or about 900 mg. In some embodiments, the inhibitor of IL-23 can be administered at a dosage of at least at most about 50 mg, about 100 mg, about 125 mg, about 150 mg, about 175 mg, about 200 mg, about 250 mg, about 300 mg, about 600 mg, about 900 mg, or about 1,500 mg

In some embodiments, the inhibitor of TNF-alpha can be administered at a dosage of at least 10 mg per individual in need thereof. In some embodiments, the inhibitor of TNF-alpha can be administered at a dosage of at least 50 mg per individual in need thereof. In some embodiments, the inhibitor of TNF-alpha can be administered at a dosage of at least 75 mg per individual in need thereof. In some embodiments, the inhibitor of TNF-alpha can be administered at a dosage of at least 80 mg per individual in need thereof. In some embodiments, the inhibitor of TNF-alpha can be administered at a dosage of at least 90 mg per individual in need thereof. In some embodiments, the inhibitor of TNF-alpha can be administered at a dosage of at least 100 mg per individual in need thereof. In some embodiments, the inhibitor of TNF-alpha can be administered at a dosage of at least 110 mg per individual in need thereof. In some embodiments, the inhibitor of TNF-alpha can be administered at a dosage of at least 120 mg per individual in need thereof. In some embodiments, the inhibitor of TNF-alpha can be administered at a dosage of at least 125 mg per individual in need thereof. In some embodiments, the inhibitor of TNF-alpha can be administered at a dosage of at least 150 mg per individual in need thereof. In some embodiments, the inhibitor of TNF-alpha can be administered at a dosage of at least 200 mg per individual in need thereof. In some embodiments, the inhibitor of TNF-alpha can be administered at a dosage of at least 1,000 mg per individual in need thereof.

In some embodiments, the inhibitor of TNF-alpha can be administered at a dosage of at least about 10 mg to about 1,000 mg. In some embodiments, the inhibitor of TNF-alpha can be administered at a dosage of at least about 10 mg to about 50 mg, about 10 mg to about 75 mg, about 10 mg to about 80 mg, about 10 mg to about 90 mg, about 10 mg to about 100 mg, about 10 mg to about 110 mg, about 10 mg to about 120 mg, about 10 mg to about 125 mg, about 10 mg to about 150 mg, about 10 mg to about 200 mg, about 10 mg to about 1,000 mg, about 50 mg to about 75 mg, about 50 mg to about 80 mg, about 50 mg to about 90 mg, about 50 mg to about 100 mg about 50 mg to about 110 mg, about 50 mg to about 120 mg, about 50 mg to about 125 mg, about 50 mg to about 150 mg, about 50 mg to about 200 mg, about 50 mg to about 1,000 mg, about 75 mg to about 80 mg, about 75 mg to about 90 mg, about 75 mg to about 100 mg, about 75 mg to about 110 mg, about 75 mg to about 120 mg, about 75 mg to about 125 mg, about 75 mg to about 150 mg, about 75 mg to about 200 mg, about 75 mg to about 1,000 mg, about 80 mg to about 90 mg, about 80 mg to about 100 mg, about 80 mg to about 110 mg, about 80 mg to about 120 mg, about 80 mg to about 125 mg, about 80 mg to about 150 mg, about 80 mg to about 200 mg, about 80 mg to about 1,000 mg, about 90 mg to about 100 mg, about 90 mg to about 110 mg, about 90 mg to about 120 mg, about 90 mg to about 125 mg, about 90 mg to about 150 mg, about 90 mg to about 200 mg, about 90 mg to about 1,000 mg, about 100 mg to about 110 mg, about 100 mg to about 120 mg, about 100 mg to about 125 mg, about 100 mg to about 150 mg, about 100 mg to about 200 mg, about 100 mg to about 1,000 mg, about 110 mg to about 120 mg, about 110 mg to about 125 mg, about 110 mg to about 150 mg, about 110 mg to about 200 mg, about 110 mg to about 1,000 mg, about 120 mg to about 125 mg, about 120 mg to about 150 mg, about 120 mg to about 200 mg, about 120 mg to about 1,000 mg, about 125 mg to about 150 mg, about 125 mg to about 200 mg, about 125 mg to about 1,000 mg, about 150 mg to about 200 mg, about 150 mg to about 1,000 mg, or about 200 mg to about 1,000 mg. In some embodiments, the inhibitor of TNF-alpha can be administered at a dosage of at least about 10 mg, about 50 mg, about 75 mg, about 80 mg, about 90 mg, about 100 mg, about 110 mg, about 120 mg, about 125 mg, about 150 mg, about 200 mg, or about 1,000 mg. In some embodiments, the inhibitor of TNF-alpha can be administered at a dosage of at least at least about 10 mg, about 50 mg, about 75 mg, about 80 mg, about 90 mg about 100 mg, about 110 mg, about 120 mg, about 125 mg, about 150 mg, or about 200 mg. In some embodiments, the inhibitor of TNF-alpha can be administered at a dosage of at least at most about 50 mg, about 75 mg, about 80 mg, about 90 mg, about 100 mg, about 110 mg, about 120 mg about 125 mg, about 150 mg, about 200 mg, or about 1,000 mg.

In some embodiments, the inhibitor of both IL-23 and TNF-alpha can be administered at a dosage of at least 15 mg per individual in need thereof. In some embodiments, the inhibitor of both IL-23 and TNF-alpha can be administered at a dosage of at least 60 mg per individual in need thereof. In some embodiments, the inhibitor of both IL-23 and TNF-alpha can be administered at a dosage of at least 100 mg per individual in need thereof. In some embodiments, the inhibitor of both IL-23 and TNF-alpha can be administered at a dosage of at least 120 mg per individual in need thereof. In some embodiments, the inhibitor of both IL-23 and TNF-alpha can be administered at a dosage of at least 130 mg per individual in need thereof. In some embodiments, the inhibitor of both IL-23 and TNF-alpha can be administered at a dosage of at least 135 mg per individual in need thereof. In some embodiments, the inhibitor of both IL-23 and TNF-alpha can be administered at a dosage of at least 140 mg per individual in need thereof. In some embodiments, the inhibitor of both IL-23 and TNF-alpha can be administered at a dosage of at least 150 mg per individual in need thereof. In some embodiments, the inhibitor of both IL-23 and TNF-alpha can be administered at a dosage of at least 170 mg per individual in need thereof. In some embodiments, the inhibitor of both IL-23 and TNF-alpha can be administered at a dosage of at least 270 mg per individual in need thereof. In some embodiments, the inhibitor of both IL-23 and TNF-alpha can be administered at a dosage of at least 1350 mg per individual in need thereof.

In some embodiments, the inhibitor of both IL-23 and TNF-alpha can be administered at a dosage of at least about 15 mg to about 1,350 mg. In some embodiments, the inhibitor of both IL-23 and TNF-alpha can be administered at a dosage of at least about 15 mg to about 60 mg, about 15 mg to about 100 mg, about 15 mg to about 120 mg, about 15 mg to about 130 mg, about 15 mg to about 135 mg, about 15 mg to about 140 mg, about 15 mg to about 150 mg, about 15 mg to about 170 mg, about 15 mg to about 270 mg, about 15 mg to about 1,350 mg, about 60 mg to about 100 mg, about 60 mg to about 120 mg, about 60 mg to about 130 mg, about 60 mg to about 135 mg, about 60 mg to about 140 mg, about 60 mg to about 150 mg, about 60 mg to about 170 mg, about 60 mg to about 270 mg, about 60 mg to about 1,350 mg, about 100 mg to about 120 mg, about 100 mg to about 130 mg, about 100 mg to about 135 mg, about 100 mg to about 140 mg, about 100 mg to about 150 mg, about 100 mg to about 170 mg, about 100 mg to about 270 mg, about 100 mg to about 1,350 mg, about 120 mg to about 130 mg, about 120 mg to about 135 mg about 120 mg to about 140 mg, about 120 mg to about 150 mg, about 120 mg to about 170 mg, about 120 mg to about 270 mg, about 120 mg to about 1,350 mg, about 130 mg to about 135 mg about 130 mg to about 140 mg, about 130 mg to about 150 mg, about 130 mg to about 170 mg, about 130 mg to about 270 mg, about 130 mg to about 1,350 mg, about 135 mg to about 140 mg about 135 mg to about 150 mg, about 135 mg to about 170 mg, about 135 mg to about 270 mg, about 135 mg to about 1,350 mg, about 140 mg to about 150 mg, about 140 mg to about 170 mg about 140 mg to about 270 mg, about 140 mg to about 1,350 mg, about 150 mg to about 170 mg about 150 mg to about 270 mg, about 150 mg to about 1,350 mg, about 170 mg to about 270 mg about 170 mg to about 1,350 mg, or about 270 mg to about 1,350 mg. In some embodiments, the inhibitor of both IL-23 and TNF-alpha can be administered at a dosage of at least about 15 mg, about 60 mg, about 100 mg, about 120 mg, about 130 mg, about 135 mg, about 140 mg, about 150 mg, about 170 mg, about 270 mg, or about 1,350 mg. In some embodiments, the inhibitor of both IL-23 and TNF-alpha can be administered at a dosage of at least at least about 15 mg, about 60 mg about 100 mg, about 120 mg, about 130 mg, about 135 mg, about 140 mg, about 150 mg, about 170 mg, or about 270 mg. In some embodiments, the inhibitor of both IL-23 and TNF-alpha can be administered at a dosage of at least at most about 60 mg, about 100 mg, about 120 mg, about 130 mg, about 135 mg, about 140 mg, about 150 mg, about 170 mg, about 270 mg, or about 1,350 mg.

In some embodiments, the JAK inhibitor can be administered at a dosage of at least 5 mg per individual in need thereof. In some embodiments, the JAK inhibitor can be administered at a dosage of at least 10 mg per individual in need thereof. In some embodiments, the JAK inhibitor can be administered at a dosage of at least 15 mg per individual in need thereof. In some embodiments, the JAK inhibitor can be administered at a dosage of at least 30 mg per individual in need thereof. In some embodiments, the JAK inhibitor can be administered at a dosage of at least 45 mg per individual in need thereof. In some embodiments, the JAK inhibitor can be administered at a dosage of at least 60 mg per individual in need thereof. In some embodiments, the JAK inhibitor can be administered at a dosage of at least 100 mg per individual in need thereof. In some embodiments, the JAK inhibitor can be administered at a dosage of at least 150 mg per individual in need thereof. In some embodiments, the JAK inhibitor can be administered at a dosage of at least 200 mg per individual in need thereof. In some embodiments, the JAK inhibitor can be administered at a dosage of at least 250 mg per individual in need thereof. In some embodiments, the JAK inhibitor can be administered at a dosage of at least 300 mg per individual in need thereof. In some embodiments, the JAK inhibitor can be administered at a dosage of at least 500 mg per individual in need thereof.

In some embodiments, the JAK inhibitor can be administered at a dosage of at least about 5 mg to about 500 mg. In some embodiments, the JAK inhibitor can be administered at a dosage of at least about 5 mg to about 10 mg, about 5 mg to about 15 mg, about 5 mg to about 30 mg, about 5 mg to about 45 mg, about 5 mg to about 60 mg, about 5 mg to about 100 mg, about 5 mg to about 150 mg, about 5 mg to about 200 mg, about 5 mg to about 250 mg, about 5 mg to about 300 mg, about 5 mg to about 500 mg, about 10 mg to about 15 mg, about 10 mg to about 30 mg, about 10 mg to about 45 mg, about 10 mg to about 60 mg, about 10 mg to about 100 mg, about 10 mg to about 150 mg, about 10 mg to about 200 mg, about 10 mg to about 250 mg, about 10 mg to about 300 mg, about 10 mg to about 500 mg, about 15 mg to about 30 mg, about 15 mg to about 45 mg, about 15 mg to about 60 mg, about 15 mg to about 100 mg, about 15 mg to about 150 mg about 15 mg to about 200 mg, about 15 mg to about 250 mg, about 15 mg to about 300 mg, about 15 mg to about 500 mg, about 30 mg to about 45 mg, about 30 mg to about 60 mg, about 30 mg to about 100 mg, about 30 mg to about 150 mg, about 30 mg to about 200 mg, about 30 mg to about 250 mg, about 30 mg to about 300 mg, about 30 mg to about 500 mg, about 45 mg to about 60 mg about 45 mg to about 100 mg, about 45 mg to about 150 mg, about 45 mg to about 200 mg, about 45 mg to about 250 mg, about 45 mg to about 300 mg, about 45 mg to about 500 mg, about 60 mg to about 100 mg, about 60 mg to about 150 mg, about 60 mg to about 200 mg, about 60 mg to about 250 mg, about 60 mg to about 300 mg, about 60 mg to about 500 mg, about 100 mg to about 150 mg, about 100 mg to about 200 mg, about 100 mg to about 250 mg, about 100 mg to about 300 mg, about 100 mg to about 500 mg, about 150 mg to about 200 mg, about 150 mg to about 250 mg, about 150 mg to about 300 mg, about 150 mg to about 500 mg, about 200 mg to about 250 mg, about 200 mg to about 300 mg, about 200 mg to about 500 mg, about 250 mg to about 300 mg, about 250 mg to about 500 mg, or about 300 mg to about 500 mg. In some embodiments, the JAK inhibitor can be administered at a dosage of at least about 5 mg, about 10 mg, about 15 mg, about 30 mg, about 45 mg, about 60 mg, about 100 mg, about 150 mg, about 200 mg, about 250 mg, about 300 mg, or about 500 mg. In some embodiments, the JAK inhibitor can be administered at a dosage of at least at least about 5 mg, about 10 mg, about 15 mg, about 30 mg, about 45 mg, about 60 mg, about 100 mg, about 150 mg, about 200 mg, about 250 mg, or about 300 mg. In some embodiments, the JAK inhibitor can be administered at a dosage of at least at most about 10 mg, about 15 mg, about 30 mg, about 45 mg, about 60 mg, about 100 mg, about 150 mg, about 200 mg, about 250 mg, about 300 mg, or about 500 mg.

In some embodiments, the S1P modulator can be administered at a dosage of at least 100 mg per individual in need thereof. In some embodiments, the S1P modulator can be administered at a dosage of at least 230 mg per individual in need thereof. In some embodiments, the S1P modulator can be administered at a dosage of at least 300 mg per individual in need thereof. In some embodiments, the S1P modulator can be administered at a dosage of at least 400 mg per individual in need thereof. In some embodiments, the S1P modulator can be administered at a dosage of at least 460 mg per individual in need thereof. In some embodiments, the S1P modulator can be administered at a dosage of at least 500 mg per individual in need thereof. In some embodiments, the S1P modulator can be administered at a dosage of at least 600 mg per individual in need thereof. In some embodiments, the S1P modulator can be administered at a dosage of at least 700 mg per individual in need thereof. In some embodiments, the S1P modulator can be administered at a dosage of at least 800 mg per individual in need thereof. In some embodiments, the S1P modulator can be administered at a dosage of at least 920 mg per individual in need thereof. In some embodiments, the S1P modulator can be administered at a dosage of at least 1,000 mg per individual in need thereof. In some embodiments, the S1P modulator can be administered at a dosage of at least 1,840 mg per individual in need thereof.

In some embodiments, the S1P modulator can be administered at a dosage of at least about 100 mg to about 1,840 mg. In some embodiments, the S1P modulator can be administered at a dosage of at least about 100 mg to about 230 mg, about 100 mg to about 300 mg, about 100 mg to about 400 mg, about 100 mg to about 460 mg, about 100 mg to about 500 mg, about 100 mg to about 600 mg, about 100 mg to about 700 mg, about 100 mg to about 800 mg, about 100 mg to about 920 mg, about 100 mg to about 1,000 mg, about 100 mg to about 1,840 mg, about 230 mg to about 300 mg, about 230 mg to about 400 mg, about 230 mg to about 460 mg, about 230 mg to about 500 mg, about 230 mg to about 600 mg, about 230 mg to about 700 mg, about 230 mg to about 800 mg, about 230 mg to about 920 mg, about 230 mg to about 1,000 mg, about 230 mg to about 1,840 mg, about 300 mg to about 400 mg, about 300 mg to about 460 mg, about 300 mg to about 500 mg, about 300 mg to about 600 mg, about 300 mg to about 700 mg, about 300 mg to about 800 mg, about 300 mg to about 920 mg, about 300 mg to about 1,000 mg, about 300 mg to about 1,840 mg, about 400 mg to about 460 mg, about 400 mg to about 500 mg, about 400 mg to about 600 mg, about 400 mg to about 700 mg, about 400 mg to about 800 mg, about 400 mg to about 920 mg, about 400 mg to about 1,000 mg, about 400 mg to about 1,840 mg, about 460 mg to about 500 mg, about 460 mg to about 600 mg, about 460 mg to about 700 mg, about 460 mg to about 800 mg, about 460 mg to about 920 mg, about 460 mg to about 1,000 mg, about 460 mg to about 1,840 mg, about 500 mg to about 600 mg, about 500 mg to about 700 mg, about 500 mg to about 800 mg, about 500 mg to about 920 mg, about 500 mg to about 1,000 mg, about 500 mg to about 1,840 mg, about 600 mg to about 700 mg, about 600 mg to about 800 mg, about 600 mg to about 920 mg, about 600 mg to about 1,000 mg, about 600 mg to about 1,840 mg, about 700 mg to about 800 mg, about 700 mg to about 920 mg, about 700 mg to about 1,000 mg, about 700 mg to about 1,840 mg, about 800 mg to about 920 mg, about 800 mg to about 1,000 mg, about 800 mg to about 1,840 mg, about 920 mg to about 1,000 mg, about 920 mg to about 1,840 mg, or about 1,000 mg to about 1,840 mg. In some embodiments, the S1P modulator can be administered at a dosage of at least about 100 mg, about 230 mg, about 300 mg, about 400 mg, about 460 mg, about 500 mg, about 600 mg, about 700 mg, about 800 mg, about 920 mg, about 1,000 mg, or about 1,840 mg. In some embodiments, the S1P modulator can be administered at a dosage of at least at least about 100 mg, about 230 mg, about 300 mg, about 400 mg, about 460 mg, about 500 mg, about 600 mg, about 700 mg, about 800 mg, about 920 mg, or about 1,000 mg. In some embodiments, the S1P modulator can be administered at a dosage of at least at most about 230 mg about 300 mg, about 400 mg, about 460 mg, about 500 mg, about 600 mg, about 700 mg, about 800 mg, about 920 mg, about 1,000 mg, or about 1,840 mg.

In some embodiments, the TL1A inhibitor can be administered at a dosage of at least 50 mg per individual in need thereof. In some embodiments, the TL1A inhibitor can be administered at a dosage of at least 100 mg per individual in need thereof. In some embodiments, the TL1A inhibitor can be administered at a dosage of at least 200 mg per individual in need thereof. In some embodiments, the TL1A inhibitor can be administered at a dosage of at least 250 mg per individual in need thereof. In some embodiments, the TL1A inhibitor can be administered at a dosage of at least 350 mg per individual in need thereof. In some embodiments, the TL1A inhibitor can be administered at a dosage of at least 400 mg per individual in need thereof. In some embodiments, the TL1A inhibitor can be administered at a dosage of at least 500 mg per individual in need thereof. In some embodiments, the TL1A inhibitor can be administered at a dosage of at least 600 mg per individual in need thereof. In some embodiments, the TL1A inhibitor can be administered at a dosage of at least 700 mg per individual in need thereof. In some embodiments, the TL1A inhibitor can be administered at a dosage of at least 750 mg per individual in need thereof. In some embodiments, the TL1A inhibitor can be administered at a dosage of at least 1,000 mg per individual in need thereof. In some embodiments, the TL1A inhibitor can be administered at a dosage of at least 1500 mg per individual in need thereof.

In some embodiments, the TL1A inhibitor can be administered at a dosage of at least about 50 mg to about 1,500 mg. In some embodiments, the TL1A inhibitor can be administered at a dosage of at least about 50 mg to about 100 mg, about 50 mg to about 200 mg, about 50 mg to about 250 mg, about 50 mg to about 350 mg, about 50 mg to about 400 mg, about 50 mg to about 500 mg, about 50 mg to about 600 mg, about 50 mg to about 700 mg, about 50 mg to about 750 mg, about 50 mg to about 1,000 mg, about 50 mg to about 1,500 mg, about 100 mg to about 200 mg, about 100 mg to about 250 mg, about 100 mg to about 350 mg, about 100 mg to about 400 mg, about 100 mg to about 500 mg, about 100 mg to about 600 mg, about 100 mg to about 700 mg, about 100 mg to about 750 mg, about 100 mg to about 1,000 mg, about 100 mg to about 1,500 mg, about 200 mg to about 250 mg, about 200 mg to about 350 mg, about 200 mg to about 400 mg, about 200 mg to about 500 mg, about 200 mg to about 600 mg, about 200 mg to about 700 mg, about 200 mg to about 750 mg, about 200 mg to about 1,000 mg, about 200 mg to about 1,500 mg, about 250 mg to about 350 mg, about 250 mg to about 400 mg, about 250 mg to about 500 mg, about 250 mg to about 600 mg, about 250 mg to about 700 mg, about 250 mg to about 750 mg, about 250 mg to about 1,000 mg, about 250 mg to about 1,500 mg, about 350 mg to about 400 mg, about 350 mg to about 500 mg, about 350 mg to about 600 mg, about 350 mg to about 700 mg, about 350 mg to about 750 mg, about 350 mg to about 1,000 mg, about 350 mg to about 1,500 mg, about 400 mg to about 500 mg, about 400 mg to about 600 mg, about 400 mg to about 700 mg, about 400 mg to about 750 mg, about 400 mg to about 1,000 mg, about 400 mg to about 1,500 mg, about 500 mg to about 600 mg, about 500 mg to about 700 mg, about 500 mg to about 750 mg, about 500 mg to about 1,000 mg, about 500 mg to about 1,500 mg, about 600 mg to about 700 mg, about 600 mg to about 750 mg, about 600 mg to about 1,000 mg, about 600 mg to about 1,500 mg, about 700 mg to about 750 mg, about 700 mg to about 1,000 mg, about 700 mg to about 1,500 mg, about 750 mg to about 1,000 mg, about 750 mg to about 1,500 mg, or about 1,000 mg to about 1,500 mg. In some embodiments, the TL1A inhibitor can be administered at a dosage of at least about 50 mg, about 100 mg, about 200 mg, about 250 mg, about 350 mg, about 400 mg, about 500 mg, about 600 mg, about 700 mg, about 750 mg, about 1,000 mg, or about 1,500 mg. In some embodiments, the TL1A inhibitor can be administered at a dosage of at least at least about 50 mg about 100 mg, about 200 mg, about 250 mg, about 350 mg, about 400 mg, about 500 mg, about 600 mg, about 700 mg, about 750 mg, or about 1,000 mg. In some embodiments, the TL1A inhibitor can be administered at a dosage of at least at most about 100 mg, about 200 mg, about 250 mg, about 350 mg, about 400 mg, about 500 mg, about 600 mg, about 700 mg, about 750 mg about 1,000 mg, or about 1,500 mg.

### Combination Therapies

A pharmaceutical composition of the invention may also comprise one or more active agents (e.g., active agents suitable for treating the diseases mentioned herein). Itis within the scope of the invention to use the pharmaceutical composition of the invention in therapeutic methods for the treatment of autoimmune diseases as an adjunct to, or in conjunction with, other established therapies normally used in the treatment of autoimmune diseases.

For the treatment of autoimmune and/or inflammatory conditions (such as Crohns disease or ulcerative colitis), possible combinations include combinations with, for example, one or more active agents selected from the list comprising: JAK inhibitors as described herein; S1P modulators as described herein; inhibitors of TL1A as described herein; 5-aminosalicylic acid, or a prodrug thereof (such as sulfasalazine, olsalazine or bisalazide); corticosteroids (e.g. prednisolone, methylprednisolone, or budesonide); immunosuppressants (e.g. cyclosporin, tacrolimus, methotrexate, azathioprine or 6-mercaptopurine); anti-TNF-alpha antibodies (e.g., infliximab, adalimumab, certolizumab pegol or golimumab); anti-IL-12/IL-23 antibodies (e.g., ustekinumab); anti-IL6R antibodies or small molecule IL-12/IL-23 inhibitors (e.g., apilimod); Anti-alpha-4-beta-7 antibodies (e.g., vedolizumab); MAdCAM-1 blockers (e.g., PF-00547659); antibodies against the cell adhesion molecule alpha-4-integrin (e.g., natalizumab); antibodies against the IL-2 receptor alpha subunit (e.g., daclizumab or basiliximab); JAK3 inhibitors (e.g., tofacitinib or R348); Syk inhibitors and prodrugs thereof (e.g., fostamatinib and R-406); Phosphodiesterase-4 inhibitors (e.g., tetomilast); HMPL-004; probiotics; Dersalazine; semapimod/CPSI-2364; and protein kinase C inhibitors (e.g. AEB-071). In certain embodiments, most suitable combination agents are infliximab, adalimumab, certolizumab pegol or golimumab.

### Non-Limiting Examples of a Treatment Regime

Described herein is a method of treating an autoimmune or inflammatory condition in an individual in need thereof, the method comprising (a) parenteral administration of a first inhibitor to the individual in need thereof, wherein the first inhibitor comprises an inhibitor of interleukin-23 (IL-23) or an inhibitor of tumor necrosis factor alpha (TNF-alpha); and (b) enteral administration of a second inhibitor to the individual in need thereof, wherein the second inhibitor comprises an inhibitor of interleukin-23 (IL-23) or an inhibitor of tumor necrosis factor alpha (TNF-alpha) thereby treating the autoimmune or inflammatory condition in the individual in need thereof.

In some embodiments, the method of treatment described herein inhibits myeloid-related processes, inflammation, epithelial homeostasis, myeloid cell activation, and inflammatory fibroblast development.

In some embodiments, the method of treatment described herein inhibits the maintenance of Th17 T cells in an individual in need thereof for Crohn's disease. In some embodiments, the method of treatment described herein inhibits the expansion of Th17 T cells in an individual in need thereof for Crohn's disease. In some embodiments, the method of treatment described herein inhibits JAK/STAT signaling in an individual in need thereof for Crohn's disease. In some embodiments, the method of treatment described herein inhibits the expansion of STAT3 and/or STAT4 signaling in an individual in need thereof for Crohn's disease. In some embodiments, the method of treatment described herein inhibits TNFR1 and/or TNFR2 signaling in an individual in need thereof for Crohn's disease. In some embodiments, the method of treatment described herein inhibits NF-κB and/or MAPK signaling in an individual in need thereof for Crohn's disease.

In some embodiments, the method of treatment described herein inhibits the maintenance of Th17 T cells in an individual in need thereof for ulcerative colitis. In some embodiments, the method of treatment described herein inhibits the expansion of Th17 T cells in an individual in need thereof for ulcerative colitis. In some embodiments, the method of treatment described herein inhibits JAK/STAT signaling in an individual in need thereof for ulcerative colitis. In some embodiments, the method of treatment described herein inhibits the expansion of STAT3 and/or STAT4 signaling in an individual in need thereof for ulcerative colitis. In some embodiments, the method of treatment described herein inhibits TNFR1 and/or TNFR2 signaling in an individual in need thereof for ulcerative colitis. In some embodiments, the method of treatment described herein inhibits NF-κB and/or MAPK signaling in an individual in need thereof for ulcerative colitis.

In some embodiments, the parenteral administration of the first inhibitor is subcutaneous or intravenous. In some embodiments, the parenteral administration of the first inhibitor is subcutaneous. In some embodiments, the parenteral administration of the first inhibitor is intravenous. In some embodiments, enteral administration of the second inhibitor is oral.

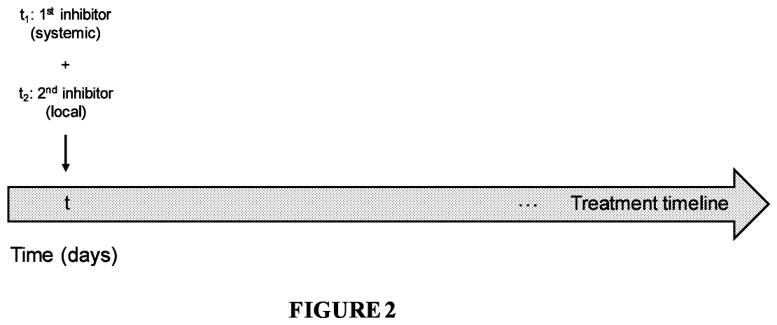

In some embodiments, the first inhibitor and the second inhibitor are administered separately. In some embodiments, the first inhibitor and the second inhibitor are administered simultaneously. A non-limiting example of a treatment regime wherein the first inhibitor and the second inhibitor are administered simultaneously is illustrated in FIG. 2.

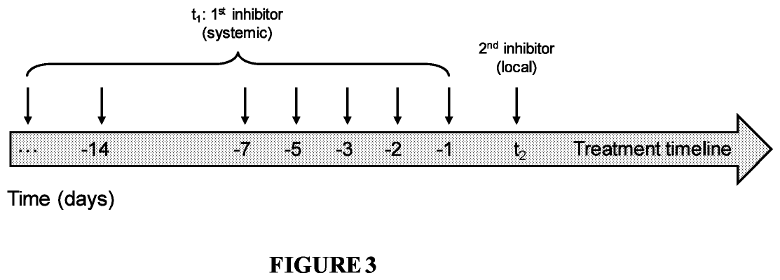

In some embodiments, the parenteral administration of the first inhibitor occurs prior to the enteral administration of the second inhibitor. A non-limiting example of a treatment regime is illustrated in FIG. 3. In some embodiments, the parenteral administration of the first inhibitor occurs prior to the enteral administration of the second inhibitor by at least 24 hours. In some embodiments, the parenteral administration of the first inhibitor occurs prior to the enteral administration of the second inhibitor by at least 2 days. In some embodiments, the parenteral administration of the first inhibitor occurs prior to the enteral administration of the second inhibitor by at least 3 days. In some embodiments, the parenteral administration of the first inhibitor occurs prior to the enteral administration of the second inhibitor by at least 7 days. In some embodiments, the parenteral administration of the first inhibitor occurs prior to the enteral administration of the second inhibitor by at least 14 days.

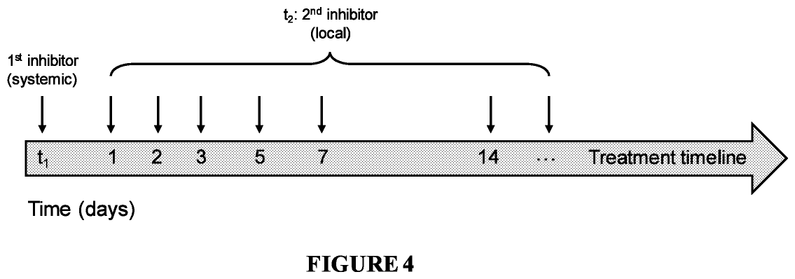

In some embodiments, the parenteral administration of the second inhibitor occurs after the enteral administration of the first inhibitor. A non-limiting example of a treatment regime is illustrated in FIG. 4. In some embodiments, the parenteral administration of the second inhibitor occurs after the enteral administration of the first inhibitor by at least 24 hours. In some embodiments, the parenteral administration of the second inhibitor occurs after the enteral administration of the first inhibitor by at least 2 days. In some embodiments, the parenteral administration of the second inhibitor after the enteral administration of the first inhibitor by at least 3 days. In some embodiments, the parenteral administration of the second inhibitor occurs after the enteral administration of the first inhibitor by at least 7 days. In some embodiments, the parenteral administration of the second inhibitor occurs after the enteral administration of the first inhibitor by at least 14 days.

In some embodiments, the first inhibitor comprises an inhibitor of IL-23. In some embodiments, the inhibitor of IL-23 comprises a small molecular inhibitor of IL-23. In some embodiments, the small molecule inhibitor of IL-23 comprises STA-5326 and peptide 2305. In some embodiments, the inhibitor of IL-23 comprises an antibody that binds IL-23 or an IL-23 binding fragment thereof. In some embodiments, the antibody that binds IL-23 or an IL-23 binding fragment thereof comprises 61 iponimod 61 abb, guselkumab, tildrakizumab, briakinumab, brazikumab, mirikizumab, ustekinumab, or combinations thereof.

In some embodiments, the first inhibitor comprises an inhibitor of TNF-alpha. In some embodiments, the inhibitor of TNF-alpha comprises a small molecule inhibitor of TNF-alpha. In some embodiments, the small molecule inhibitor of TNF-alpha comprises SAR441566, TIM1, TIM1c, SPD-304, MYMD-1. In some embodiments, the inhibitor of TNF-alpha comprises an antibody that binds TNF-alpha or a TNF-alpha binding fragment thereof. In some embodiments, the antibody that binds TNF-alpha or a TNF-alpha binding fragment thereof comprises infliximab, adalimumab, certolizumab pegol, golimumab, or combinations thereof. In some embodiments, the inhibitor of TNF-alpha comprises a soluble TNF-alpha receptor. In some embodiments, the soluble TNF-alpha receptor comprises etanercept.

In some embodiments, the first inhibitor comprises an inhibitor of IL-23 and an inhibitor of TNF-alpha. In some embodiments, the inhibitor of IL-23 and an inhibitor of TNF-alpha are administered separately. In some embodiments, the inhibitor of IL-23 and an inhibitor of TNF-alpha are administered simultaneously.

In some embodiments, the second inhibitor comprises an inhibitor of IL-23. In some embodiments, the inhibitor of IL-23 comprises an antibody that binds IL-23 or an IL-23 binding fragment thereof. In some embodiments, the antibody that binds IL-23 or an IL-23 binding fragment thereof comprises a VHH. In some embodiments, the VHH comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9. In some embodiments, the VHH comprises an amino acid sequence at least about 85%, 90%, 95%, 97%, 98%, or 99% identical to that set forth in any one of SEQ ID NOs: 10-12.

In some embodiments, the second inhibitor comprises an inhibitor of TNF-alpha. In some embodiments, the inhibitor of TNF-alpha comprises an antibody that binds TNF-alpha or a TNF-alpha binding fragment thereof. In some embodiments, the antibody that binds TNF-alpha or an TNF-alpha binding fragment thereof comprises a VHH. In some embodiments, the VHH comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the VHH comprises an amino acid sequence at least about 85%, 90%, 95%, 97%, 98%, or 99% identical to that set forth in any one of SEQ ID NOs: 118 or 125.

In some embodiments, the first inhibitor and the second inhibitor is an inhibitor of IL-23 and TNF-alpha. In some embodiments, the inhibitor of IL-23 and the inhibitor of TNF-alpha comprises a polypeptide that binds to both IL-23 and TNF-alpha. In some embodiments, the polypeptide that binds to both IL-23 and TNF-alpha comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID 10-12; and wherein the second binding region comprises an amino acid sequence at least about 85%, 90%, 95%, 97%, 98%, or 99% identical to that set forth in any one of SEQ ID NOs: 118 or 125. In some embodiments, the first binding region and the second binding region are coupled by a protease liable linker.

The method of treatment described herein can further comprise administration of a third inhibitor. In some embodiments, the method of treatment described herein further comprises administration of a third inhibitor wherein the third inhibitor comprises an inhibitor of IL-23 or an inhibitor of TNF-alpha. In some embodiments, administration of the third inhibitor is enteral. In some embodiments, administration of the third inhibitor is oral. In some embodiments, the enteral administration of the third inhibitor occurs prior to the enteral administration of the second inhibitor. In some embodiments, the enteral administration of the third inhibitor occurs prior to the enteral administration of the second inhibitor by at least 24 hours. In some embodiments, the enteral administration of the third inhibitor occurs prior to the enteral administration of the second inhibitor by at least 2 days. In some embodiments, the enteral administration of the third inhibitor occurs prior to the enteral administration of the second inhibitor by at least 3 days. In some embodiments, the enteral administration of the third inhibitor occurs prior to the enteral administration of the second inhibitor by at least 7 days. In some embodiments, the enteral administration of the third inhibitor occurs prior to the enteral administration of the second inhibitor by at least 14 days.

In some embodiments, the third inhibitor comprises an inhibitor of IL-23. In some embodiments, the inhibitor of IL-23 comprises an antibody that binds IL-23 or an IL-23 binding fragment thereof. In some embodiments, the antibody that binds IL-23 or an IL-23 binding fragment thereof comprises a VHH. In some embodiments, the VHH comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9. In some embodiments, the VHH comprises an amino acid sequence at least about 85%, 90%, 95%, 97%, 98%, or 99% identical to that set forth in any one of SEQ ID NOs: 10-12.

In some embodiments, the third inhibitor comprises an inhibitor of TNF-alpha. In some embodiments, the inhibitor of TNF-alpha comprises an antibody that binds TNF-alpha or a TNF-alpha binding fragment thereof. In some embodiments, the antibody that binds TNF-alpha or an TNF-alpha binding fragment thereof comprises a VHH. In some embodiments, the VHH comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the VHH comprises an amino acid sequence at least about 85%, 90%, 95%, 97%, 98%, or 99% identical to that set forth in any one of SEQ ID NOs: 118 or 125.

In some embodiments, the third inhibitor is an inhibitor of IL-23 and TNF-alpha. In some embodiments, the inhibitor of IL-23 and the inhibitor of TNF-alpha comprises a polypeptide that binds to both IL-23 and TNF-alpha. In some embodiments, the polypeptide that binds to both IL-23 and TNF-alpha comprises a first binding region that binds to IL-23 and a second binding region that binds to TNF-alpha, wherein the first binding region that binds IL-23 comprises: a) a CDR1 comprising an amino acid sequence as set forth in any one of SEQ ID NOs: 1, 4, or 7; b) a CDR2 comprising an amino acid sequence as set forth in any one of SEQ ID NOs: 2, 5, or 8; and c) a CDR3 comprising an amino acid sequence as set forth in any one of SEQ ID NOs: 3, 6, or 9; wherein the second binding region that binds TNF-alpha comprises: a) a CDR1 comprising an amino acid sequence as set forth in any one of SEQ ID NOs: 101, 107, 108, or 119; b) a CDR2 comprising an amino acid sequence as set forth in any one of SEQ ID NOs: 102, 109-117, or 120; and c) a CDR3 comprising an amino acid sequence as set forth in any one of SEQ ID NOs: 103-106 or 121-124. In some embodiments, the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125. In some embodiments, the first binding region and the second binding region are coupled by a protease liable linker.

### EXAMPLES

The following illustrative examples are representative of embodiments of compositions and methods described herein and are not meant to be limiting in any way.

TABLE 1
Amino acid sequences or polynucleotide sequences disclosed are represented herein.

| SEQ ID NO: | Sequence | Description |
|---|---|---|
| 1 | FKVMG | IL-23: ID-L253T CDR1 |
| 2 | TITTGGSPDYSDSVKG | IL-23: ID-L253T CDR2 |
| 3 | RLWAHITSSGHDV | IL-23: ID-L253T CDR3 |
| 4 | GSAMG | IL-23: 10E2 CDR1 |
| 5 | VIIWTGGYTYYADSVK | IL-23: 10E2 CDR2 |
| 6 | RTPNSSWFPTTLYEYD | IL-23: 10E2 CDR3 |
| 7 | NYIMG | IL-23: 10G10 CDR1 |
| 8 | IITANGVATDYRDSVK | IL-23: 10G10 CDR2 |
| 9 | RTRSGSLEDPGTFDY | IL-23: 10G10 CDR3 |
| 10 | DVQLVESGGGQVQPGGSLSLSCESSVSIWSFKVMGWFRQ | IL-23: ID-L253T |
| | VPGKQRELVATITTGGSPDYSDSVKGRFTISRDYDKRTL | |
| | YLQMNSLKPEDTAVYYCAGRLWAHITSSGHDVWGQGTQV | |
| | TVSS | |
| 11 | EVQLVESGGGLAQAGGSLRLSCAPSGRAFSGSAMGWFRQ | IL-23: 10E2 |
| | VPGKEREFVGVIIWTGGYTYYADSVKGRFTISRDSAKNT | |
| | VYLQMDSLKPEDTAVYYCAARTPNSSWFPTTLYEYDYWG | |
| | QGTQVTVSS | |
| 12 | EVQLVESGGGLVQAGTSLRLSCAASGRTFSNYIMGWFRQ | IL-23: 10G10 |
| | APGKEREFVAIITANGVATDYRDSVKGRETISRDNARNT | |
| | GYLEMNSLKPEDTAVYFCALRTRSGSLFDPGTFDYWGQG | |
| | TQVTVSS | |
| 13 | EVQLVESGGGQVQPGGSLSLSCESSVSIWSFKVMGWFRQ | IL-23: D1E ID-L253T |
| | VPGKQRELVATITTGGSPDYSDSVKGRFTISRDYDKRTL | |
| | YLQMNSLKPEDTAVYYCAGRLWAHITSSGHDVWGQGTQV | |
| | TVSS | |
| 101 | SHWMY | TNF-alpha: ID-38F CDR1 |
| 102 | EINTNGLITHYGDSVK | TNF-alpha: ID-38F CDR2 |
| 103 | NQHGLN | TNF-alpha: ID-38F CDR3 |
| 104 | NQKGLN | TNF-alpha: Q65F2 CDR3 |
| 105 | NQMGLN | TNF-alpha: Q65F1 CDR3 |
| 106 | NERGLN | TNF-alpha: Q65D3 CDR3 |
| 107 | VHWMY | TNF-alpha: Q65F1 CDR1 |
| 108 | NHWMC | TNF-alpha: Q65D1 CDR1 |
| 109 | EINTNGLITHYGDSVH | TNF-alpha: ID27F CDR2 |
| 110 | EINTNGLITKYGDSVH | TNF-alpha: ID28F CDR2 |
| 111 | EINTNGLITSYVDSVK | TNF-alpha: Q65F2 CDR2 |
| 112 | EINTNGLITKYIDSVR | TNF-alpha: Q65F3 CDR2 |
| 113 | EINTNGLITNYVDSVK | TNF-alpha: Q62F2 CDR2 |
| 114 | EINTNGLITKYIDSVG | TNF-alpha: Q65F1 CDR2 |
| 115 | EINTNGLITKYADFVK | TNF-alpha: Q65D1 CDR2 |
| 116 | EINTNGLITKYADSTK | TNF-alpha: Q65D3 CDR2 |
| 117 | EINTNGLITKYGDSVK | TNF-alpha: Q65B1 CDR2 |
| 118 | DVQLVESGGGLVQPGGSLKLSCAASGFDFSSHWMYWVRQ | TNF-alpha: ID-38F |
| | APGKELEWLSEINTNGLITHYGDSVKGRFTVSRNNAANK | |
| | MYLELTRLEPEDTALYYCARNQHGLNKGQGTQVTVSS | |
| 119 | IHWMY | TNF-alpha: Q62E10 CDR1 |
| 120 | EINTNALITKYADSVK | TNF-alpha: Q62E10 CDR2 |
| 121 | TQNGAA | TNF-alpha: Q62E10 CDR3 |
| 122 | SRNGAA | TNF-alpha: Q65F6 CDR3 |
| 123 | ARNGAA | TNF-alpha: Q65F11 CDR3 |
| 124 | TQNGKT | TNF-alpha: Q62F 10 CDR3 |
| 125 | QVQLVESGGGLVQPGGSLRLSCTTSGLDFGIHWMYWFRQ | TNF-alpha: Q62E10 |
| | APGKELEWVAEINTNALITKYADSVKGRFTISRDNAKNT | |
| | LFLQMNDLKSEDTAVYYCSNTQNGAAKGQGVQVTVSS | |
| 201 | DVQLVESGGGLVQPGGSLKLSCAASGFDFSSHWMYWVRQ | TNF-alpha/IL-23 dual |
| | APGKELEWLSEINTNGLITHYGDSVKGRFTVSRNNAANK | inhibitor: FA1K |
| | MYLELTRLEPEDTALYYCARNQHGLNKGQGTQVTVSSGG | |
| | GGSGGGGSKGGGGSGGGGSEVQLVESGGGQVQPGGSLSL | |
| | SCESSVSIWSFKVMGWFRQVPGKQRELVATITTGGSPDY | |
| | SDSVKGRFTISRDYDKRTLYLQMNSLKPEDTAVYYCAGR | |
| | LWAHITSSGHDVWGQGTQVTVSS | |
| 202 | DVQLVESGGGQVQPGGSLSLSCESSVSIWSFKVMGWFRQ | IL-23/TNF-alpha dual |
| | VPGKQRELVATITTGGSPDYSDSVKGRFTISRDYDKRTL | inhibitor: FA1K-based |
| | YLQMNSLKPEDTAVYYCAGRLWAHITSSGHDVWGQGTQV | |
| | TVSSGGGGSGGGGSKGGGGSGGGGSEVQLVESGGGLVQP | |
| | GGSLKLSCAASGFDFSSHWMYWVRQAPGKELEWLSEINT | |
| | NGLITHYGDSVKGRFTVSRNNAANKMYLELTRLEPEDTA | |
| | LYYCARNQHGLNKGQGTQVTVSS | |
| 203 | DVQLVESGGGLVQPGGSLKLSCAASGFDFSSHWMYWVRQ | TNF-alpha fragment of |
| | APGKELEWLSEINTNGLITHYGDSVKGRFTVSRNNAANK | cleaved TNF-alpha/IL-23 |
| | MYLELTRLEPEDTALYYCARNQHGLNKGQGTQVTVSSGG | dual inhibitor |
| | GGSGGGGSK | |
| 204 | GGGGSGGGGSEVQLVESGGGQVQPGGSLSLSCESSVSIW | IL-23 fragment of cleaved |
| | SFKVMGWFRQVPGKQRELVATITTGGSPDYSDSVKGRFT | TNF-alpha/IL-23 dual |
| | ISRDYDKRTLYLQMNSLKPEDTAVYYCAGRLWAHITSSG | inhibitor |
| | HDVWGQGTQVTVSS | |
| 205 | DVQLVESGGGQVQPGGSLSLSCESSVSIWSFKVMGWFRQ | IL-23 fragment of cleaved |
| | VPGKQRELVATITTGGSPDYSDSVKGRFTISRDYDKRTL | IL-23/TNF-alpha dual |
| | YLQMNSLKPEDTAVYYCAGRLWAHITSSGHDVWGQGTQV | inhibitor |
| | TVSSGGGGSGGGGSK | |
| 206 | GGGGSGGGGSEVQLVESGGGLVQPGGSLKLSCAASGFDF | TNF-alpha fragment of |
| | SSHWMYWVRQAPGKELEWLSEINTNGLITHYGDSVKGRF | cleaved IL-23/TNF-alpha |
| | TVSRNNAANKMYLELTRLEPEDTALYYCARNQHGLNKGQ | dual inhibitor |
| | GTQVTVSS | |
| 301 | GGGGSKGGGGS | Linker |
| 302 | GGGGSGGGGSKGGGGSGGGGS | Linker |

### Example 1: Binding Efficacy of a Cleavable Bispecific Anti-TNF-Alpha/Anti-IL-23 Domain Antibody and its Monomeric Cleavage Products after Exposure to Trypsin

TNF-Alpha/IL-23 Dual Inhibitor can Inhibit TNF-Alpha and IL-23 Activity while Intact and after Trypsin Cleavage, and its Liberated Monomer Arms are Functional and Resistant to Human Fecal Proteases.

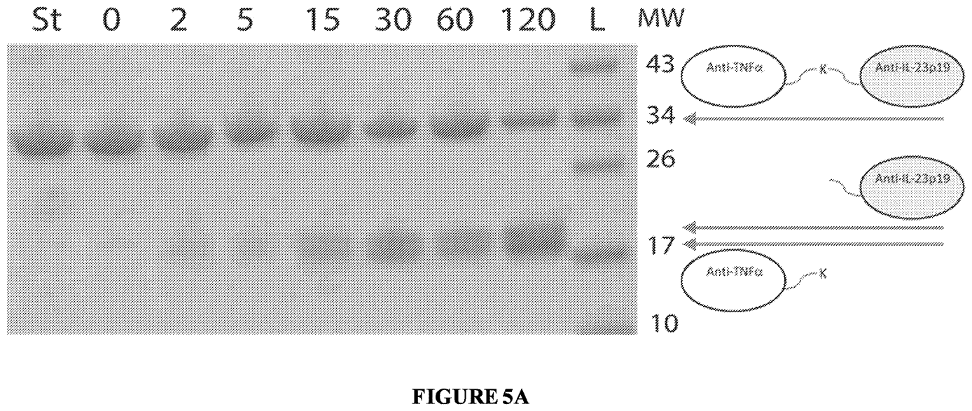

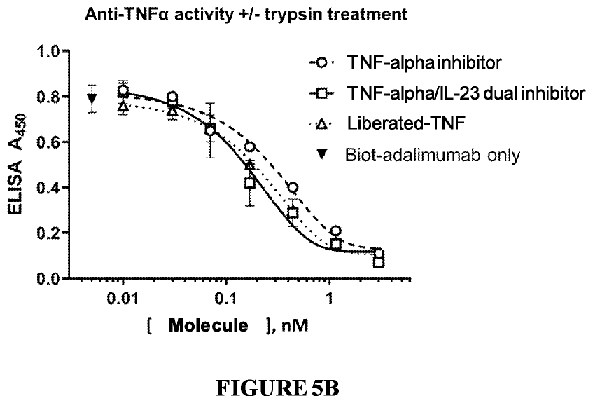

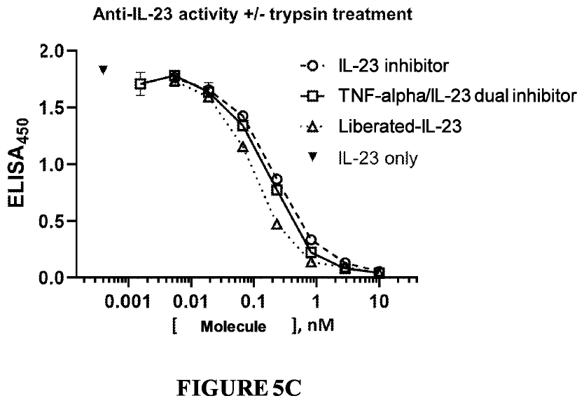

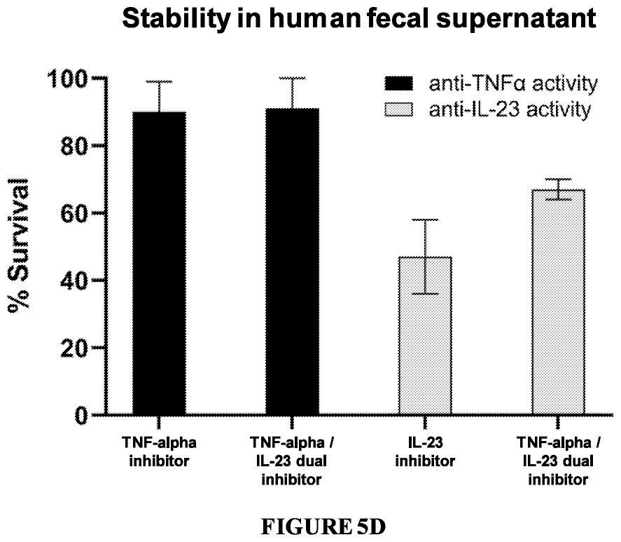

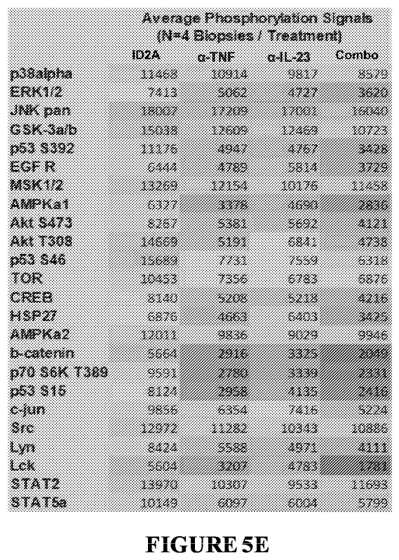

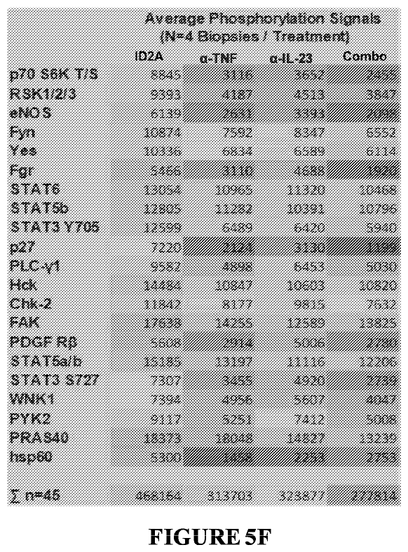

The target binding efficacy of a bispecific anti-TNF-alpha/anti-IL-23 domain antibody (SEQ ID NO: 201; hereinafter in the examples, “TNF-alpha/IL-23 dual inhibitor”) and its monomeric arms were evaluated after exposure to trypsin. TNF-alpha/IL-23 dual inhibitor contains two humanized single domain antibodies targeting TNF-alpha (SEQ ID NO: 118) and IL-23p19 (SEQ ID NO: 13) connected by a trypsin-labile linker (SEQ ID NO: 302), which can enable monomer separation within the small intestine. The anti-TNF-alpha single domain antibody is hereinafter referred to in the examples as “TNF-alpha inhibitor” and the anti-IL-23 single domain antibody is hereinafter referred to in the examples as “IL-23 inhibitor”. The central lysine linker of TNF-alpha/IL-23 dual inhibitor can be cleaved in intestinal supernatants (FIG. 5A), yielding cleavage products comprising a liberated anti-TNF-alpha monomer arm (SEQ ID NO: 203; hereinafter in the examples, “liberated-TNF”) and a liberated anti-IL-23 monomer arm (SEQ ID NO: 204; hereinafter in the examples, “liberated-IL-23”).

TNF-alpha inhibitor, TNF-alpha/IL-23 dual inhibitor, and IL-23 inhibitor were produced and purified from yeast. TNF-alpha/IL-23 dual inhibitor was incubated in pooled human fecal supernatant (HFS), and TNF-alpha/IL-23 dual inhibitor-liberated monomer arms were purified from the digestive mixture. Inhibition curves of TNF-alpha inhibitor, TNF-alpha/IL-23 dual inhibitor, IL-23 inhibitor, and trypsin-liberated TNF-alpha/IL-23 dual inhibitor monomer arms were assessed by ELISAs whereby inhibitor molecules competed with biotinylated adalimumab (anti-TNF-alpha IgG1) or interrupted IL-23/IL-23-receptor binding. The competitive ELISA data indicate that TNF-alpha/IL-23 dual inhibitor and liberated monomer arms retain full anti-TNF-alpha and anti-IL-23 activity after exposure to trypsin (FIG. 5B-5C). The competitive ELISA data also show that the liberated anti-IL-23 monomer arm, liberated-IL-23, can be more potent than the anti-IL-23 single domain antibody (IL-23 inhibitor) parent (FIG. 5C and Table 2).

TABLE 2
IC₅₀ values of TNF-alpha/IL-23 dual inhibitor,
liberated-IL-23, and liberated-TNF.

| Analyte | Method | IC₅₀ |
|---|---|---|
| TNF-alpha/ | IL-23 \| IL-23 Receptor | 0.1807 |
| IL-23 dual inhibitor | Interference ELISA | |
| Liberated-IL-23 | IL-23 \| IL-23 Receptor | 0.1029 |
| | Interference ELISA | |
| IL-23 inhibitor | IL-23 \| IL-23 Receptor | 0.2157 |
| | Interference ELISA | |

The stability of TNF-alpha/IL-23 dual inhibitor and its monomer arms post-cleavage were also evaluated. TNF-alpha/IL-23 dual inhibitor and the parent monomers, TNF-alpha inhibitor and IL-23 inhibitor, were incubated in pooled human fecal supernatant (HFS) for 4 hours. This timepoint was selected for accurate observation of differences in stability. Time 0 hour and 4 hour samples were compared for anti-TNF-alpha activity in the biotinylated adalimumab assay (TNF-alpha inhibitor and TNF-alpha/IL-23 dual inhibitor) or anti-IL-23 activity in the IL-23/IL-23R ELISA (IL-23 inhibitor and TNF-alpha/IL-23 dual inhibitor). The remaining activity in each sample at 4 hours was calculated as a survival percentage against the 0 hour time point (FIG. 5D). The assay results show that the TNF-alpha/IL-23 dual inhibitor and its cleavage products are resistant to human fecal proteases as evidenced by retention of functional activity after exposure to HFS.

The effect of TNF-alpha and IL-23 inhibition, alone and in combination, was also evaluated using ex vivo cultures of ulcerative colitis (UC) colonic human biopsies. Biopsies were collected from four different UC patients and incubated for 24 hours with single domain antibody treatments (Control (ID2A) 225 nM; TNF-alpha inhibitor 75 nM; IL-23 inhibitor 150 nM; or TNF-alpha inhibitor 75 nM+IL-23 inhibitor 150 nM combined) and analyzed for the extent of phosphorylation of tyrosine kinase receptors and signalling proteins that can be increased in inflamed intestinal tissue (FIG. 5E and FIG. 5F). Lysates were analyzed on R&D proteome profiler human phosphokinase arrays and phosphointensity data were averaged for each treatment. Heatmap shadings were applied relative to the averaged signal of each phosphoprotein in the final array data set. Total phosphorylation values were calculated by summing the averaged spot intensities of all 45 analytes. The data indicate that treatment with TNF-alpha inhibitor or IL-23 inhibitor inhibited tissue phosphoprotein levels alone and, to a greater extent, in combination (FIG. 5E and FIG. 5F).

### Example 2: Cynomolgus Monkey In Vivo Pharmacokinetics Study of a Bispecific Anti-TNF-Alpha/Anti-IL-23 Domain Antibody for the Treatment of Inflammatory Bowel Disease

### The Labile Peptide Linker in TNF-Alpha/IL-23 Dual Inhibitor is Functional and Both Dual Inhibitor-Liberated TNF-Alpha and IL-23 Monomer Arms are Stable in the Intestinal Environment of Non-Human Primates.

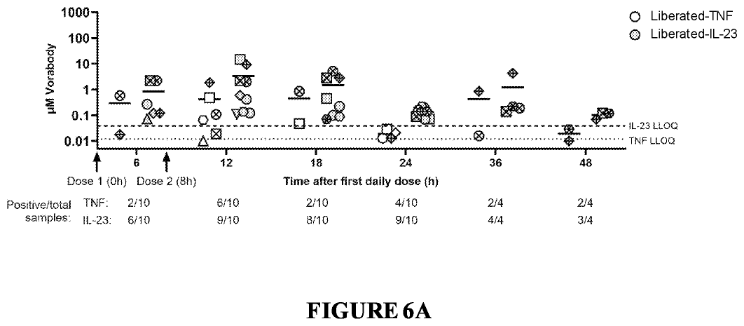

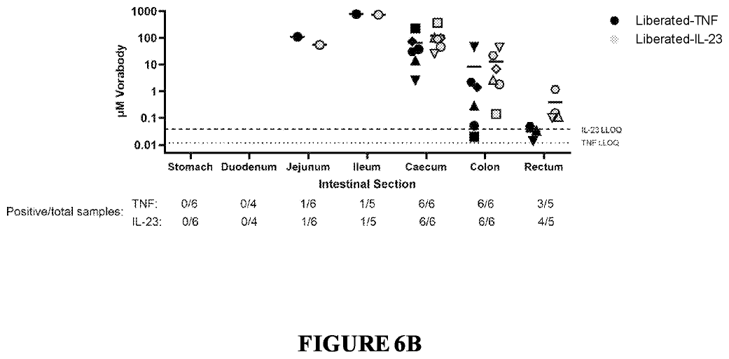

TNF-alpha/IL-23 dual inhibitor was produced and purified from yeast. 10 non-human primates (NHPs), 5 male and 5 female cynomolgus monkeys, were dosed orally twice a day for 42 days (135 mg/dose, 270 mg per day total). On Day 42, fecal samples and intestinal samples were collected and analyzed for the presence of TNF-alpha/IL-23 dual inhibitor, the TNF-alpha/IL-23 dual inhibitor-liberated anti-TNF-alpha monomer arm (“liberated-TNF”), and the liberated anti-IL-23 monomer arm (“liberated-IL-23”). On Day 42, fecal samples were collected at 6-hour timepoints following the first daily dose of TNF-alpha/IL-23 dual inhibitor for 10 animals during the first 24 hours after dosing and for 4 animals for an additional 24 hours (two 12-hour sample collection time points; FIG. 6A and Table 3). Feces produced within each time window was collected at the specified time points and treated as a single sample. The data show that both monomers were detected in ≥90% of animals for at least 1 time point on Day 42 (liberated-TNF range: 0.01-1.8 μM and liberated-IL-23 range: 0.07-14.7 μM; FIG. 6A). The data also show that 9/10 animals were positive for liberated-TNF at least one time point and that 10/10 animals were positive for liberated-IL-23 at least one time point (Table 3).

TABLE 3
Summary of day 42 fecal concentrations (Mean, μM).

| Analyte | 6 hours | 12 hours | 18 hours | 24 hours | 36 hours | 48 hours |
|---|---|---|---|---|---|---|
| Liberated-TNF | 0.2984 | 0.4217 | 0.4511 | 0.019 | 0.4347 | 0.0195 |
| Liberated-IL-23 | 0.8303 | 3.2916 | 1.4666 | 0.1384 | 1.205 | 0.1033 |
| Total samples | 10 | 10 | 10 | 10 | 4 | 4 |
| Samples positive for Liberated-TNF | 2 | 6 | 2 | 4 | 2 | 2 |
| Samples positive for Liberated-IL-23 | 6 | 9 | 8 | 9 | 4 | 3 |

6 were given a third oral dose of TNF-alpha/IL-23 dual inhibitor at the 24-hour time point and culled 4 hours after this final dose. Intestinal contents were collected from 7 intestinal sections (stomach, duodenum, jejunum, ileum, caecum, colon and rectum) at this 4-hour time point where concentrations of liberated-TNF and liberated-IL-23 were quantified by ELISA (FIG. 6B and Table 4). Intact TNF-alpha/IL-23 dual inhibitor levels were below the assay lower limit of quantitation (245 nM) in all intestinal samples, indicating that the trypsin-labile linker in TNF-alpha/IL-23 dual inhibitor functions as designed in the intestinal environment. The data indicate that all animals showed delivery of active drug after reaching caecal compartment (liberated-TNF range in caecum: 2.6-227 μM and liberated-IL-23 range in caecum: 25-369 μM; FIG. 6B). The data show that the drug concentration can decrease as molecules move from the caecum to the rectum (FIG. 6B). The data also show that 6/6 animals were positive for both monomers in caecal and colon compartments (Table 4).

TABLE 4
Summary of day 42 intestinal concentrations (Mean, μM).

| Analyte | Stomach | Duodenum | Jejunum | Ileum | Caecum | Colon | Rectum |
|---|---|---|---|---|---|---|---|
| Liberated-TNF | | | 110 | 780 | 64.2667 | 8.293 | 0.032 |
| Liberated-IL-23 | | | 55 | 742 | 121.89 | 12.9145 | 0.3825 |
| Total samples | 6 | 4 | 6 | 5 | 6 | 6 | 5 |
| Samples positive for Liberated-TNF | 0 | 0 | 1 | 1 | 6 | 6 | 3 |
| Samples positive for Liberated-IL-23 | 0 | 0 | 1 | 1 | 6 | 6 | 4 |

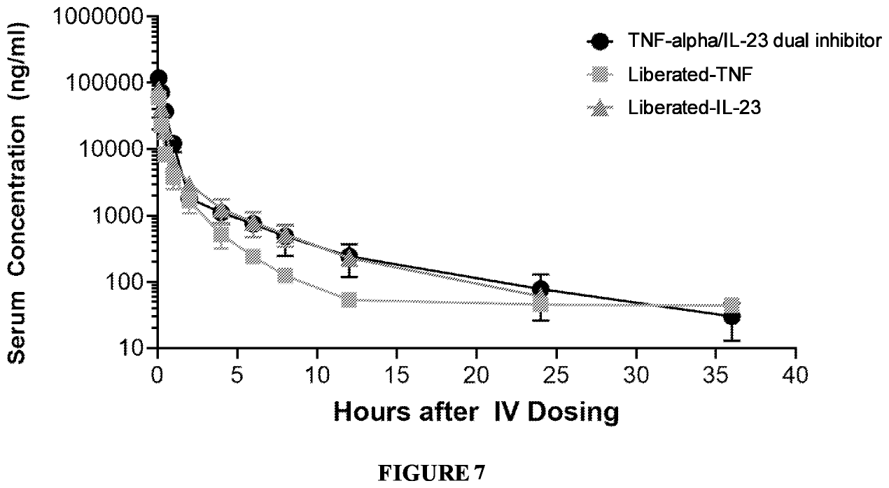

To evaluate systemic exposure of TNF-alpha/IL-23 dual inhibitor after oral dosing NHPs (n=3 per group) were intravenously administered TNF-alpha/IL-23 dual inhibitor or a mixture of liberated-TNF and liberated-IL-23. The serum concentration of each molecule was measured for the first 36 hours post-administration by ELISA (FIG. 7). Systemic exposure after oral dosing of TNF-alpha/IL-23 dual inhibitor can be low and transient since TNF-alpha/IL-23 dual inhibitor monomers have a short elimination half-life from serum after single or multiple IV doses to cynomolgus monkeys (NHPs).

### Example 3: Human Clinical Study of Orally Delivered Bispecific Anti-TNF-Alpha/Anti-IL-23 Domain Antibody for the Treatment of Inflammatory Bowel Disease

Orally Administered TNF-Alpha/IL-23 Dual Inhibitor is Safe and Well Tolerated, Selective to the Gut, and Efficiently Cleaved after Oral Dosing.

The safety, tolerability, and pharmacokinetics of TNF-alpha/IL-23 dual inhibitor when orally delivered for the treatment of IBD was evaluated in a three-part Phase 1 clinical study. TNF-alpha/IL-23 dual inhibitor can inhibit TNF-alpha and IL-23 activity while intact and after trypsin cleavage. TNF-alpha/IL-23 dual inhibitor single domain antibodies were engineered for high stability among intestinal and inflammatory proteases to enable oral dosing. Healthy subjects aged 18 to 55 years (n=42) were enrolled in randomized, double-blinded, placebo-controlled studies to evaluate single ascending doses (SAD) and multiple doses (MD) of TNF-alpha/IL-23 dual inhibitor in Parts 1 and 2 of the clinical study, respectively.

Part 1 of the clinical study evaluated the safety and tolerability of SAD of TNF-alpha/IL-23 dual inhibitor and the concentration of intact TNF-alpha/IL-23 dual inhibitor and its monomers (liberated-TNF and dual-IL-23) in serum and feces. Healthy subjects were randomized 6:2 to active and placebo treatment into one of 4 ascending dose cohorts receiving a single oral (PO) TNF-alpha/IL-23 dual inhibitor dose of 135 mg, 405 mg, 1215 mg, or 3645 mg, or placebo. Safety and tolerability, fecal and serum concentrations of TNF-alpha/IL-23 dual inhibitor and its monomers were assessed at predetermined times via antigen-binding ELISA assays. 7 adverse events (AE) of mild or moderate severity observed in Part 1. One AE of mild intensity may be treatment-related was observed in the lowest dose cohort. TNF-alpha/IL-23 dual inhibitor and its monomers were not detected in serum. Fecal concentrations of active TNF-alpha/IL-23 dual inhibitor monomers were detected at all dose levels, peaking on Day 2 and 3 post-dose, with generally higher concentrations observed in higher-dose cohorts (Tables 5-12). The highest fecal concentrations were observed in the third dose cohort on Day 2 post-dose (liberated-TNF≈260 μg/mL; dual-IL-23≈130 μg/mL) (Tables 5-12). Monomer fecal concentrations were observed in all dose cohorts in Part 1 and generally increased with each ascending dose, peaking on Day 2 and 3 after administration (Tables 5-12).

TABLE 5
Summary fecal concentrations of liberated-TNF following
a single dose of TNF-alpha/IL-23 dual inhibitor (135 mg).

| | | | Visit | | | | | |
|---|---|---|---|---|---|---|---|---|
| | | | D-1: Pre | D1: Post | D2 | D3 | D4: 72 Hr | Unsch 1 |
| Cohort | Analyte | | Concentration (ng/mL) | | | | | |
| 1 | Liberated-TNF | nObs | 7 | 5 | 5 | 5 | 8 | 1 |
| | | N(<BLQ) | 6 | 3 | 3 | 3 | 6 | 1 |
| | | Mean | 440 | 1400 | 16600 | 685 | 1860 | NR |
| | | Min | 440 | 505 | 8500 | 685 | 515 | NR |
| | | Median | 440 | 1400 | 16600 | 685 | 1860 | NR |
| | | Max | 440 | 2290 | 24750 | 685 | 3205 | NR |

NR—No result

TABLE 6
Summary fecal concentrations of liberated-TNF following
a single dose of TNF-alpha/IL-23 dual inhibitor (405 mg).

| | | | Visit | | | | | |
|---|---|---|---|---|---|---|---|---|
| | | | D-1: Pre | D1: Post | D2 | D3 | D4: 72 Hr | Unsch 1 |
| Cohort | Analyte | | Concentration (ng/mL) | | | | | |
| 2 | Liberated-TNF | nObs | 5 | 3 | 7 | 7 | 7 | NR |
| | | N(<BLQ) | 4 | 3 | 4 | 2 | 4 | NR |
| | | Mean | 430 | NR | 625 | 510 | 685 | NR |
| | | Min | 430 | NR | 525 | 415 | 475 | NR |
| | | Median | 430 | NR | 550 | 515 | 580 | NR |
| | | Max | 430 | NR | 805 | 645 | 1000 | NR |

NR—No result

TABLE 7
Summary fecal concentrations of liberated-TNF following a
single dose of TNF-alpha/IL-23 dual inhibitor (1215 mg).

| | | | Visit | | | | | |
|---|---|---|---|---|---|---|---|---|
| | | | D-1: Pre | D1: Post | D2 | D3 | D4: 72 Hr | Unsch 1 |
| Cohort | Analyte | | Concentration (ng/mL) | | | | | |
| 3 | Liberated-TNF | nObs | 1 | 4 | 6 | 7 | 8 | 2 |
| | | N(<BLQ) | 0 | 4 | 2 | 3 | 7 | 0 |
| | | Mean | 410 | NR | 187000 | 19100 | 625 | 53000 |
| | | Min | 410 | NR | 49450 | 430 | 625 | 10350 |
| | | Median | 410 | NR | 248000 | 945 | 625 | 53000 |
| | | Max | 410 | NR | 263500 | 74000 | 625 | 96000 |

NR—No result

TABLE 8
Summary fecal concentrations of liberated-TNF following a
single dose of TNF-alpha/IL-23 dual inhibitor (3645 mg).

| | | | Visit | | | | | |
|---|---|---|---|---|---|---|---|---|
| | | | D-1: Pre | D1: Post | D2 | D3 | D4: 72 Hr | Unsch 1 |
| Cohort | Analyte | | Concentration (ng/mL) | | | | | |
| 4 | Liberated-TNF | nObs | 3 | 1 | 4 | 4 | 7 | 2 |
| | | N(<BLQ) | 3 | 1 | 3 | 2 | 5 | 0 |
| | | Mean | NR | NR | 17550 | 2615 | 4280 | 2075 |
| | | Min | NR | NR | 17550 | 1860 | 4280 | 415 |
| | | Median | NR | NR | 17550 | 2615 | 4280 | 2075 |
| | | Max | NR | NR | 17550 | 3365 | 4280 | 3735 |

NR—No result

TABLE 9
Summary fecal concentrations of liberated-IL-23 following
a single dose of TNF-alpha/IL-23 dual inhibitor (135 mg).

| | | Visit | | | | | |
|---|---|---|---|---|---|---|---|
| Analyte: | | D-1: | D1: | | | D4: | Unsch |
| Liberated-IL23 | | Pre | Post | D2 | D3 | 72 Hr | 1 |
| Cohort | | Concentration (ng/mL) | | | | | |
| Cohort 1 | nObs | 7 | 5 | 5 | 5 | 8 | 1 |
| | N(<BLQ) | 7 | 4 | 2 | 3 | 8 | 1 |
| | Mean | NR | 1705 | 4160 | 1765 | NR | NR |
| | Min | NR | 1705 | 545 | 620 | NR | NR |
| | Median | NR | 1705 | 885 | 1765 | NR | NR |
| | Max | NR | 1705 | 11050 | 2915 | NR | NR |

NR—No result

TABLE 10
Summary fecal concentrations of liberated-IL-23 following
a single dose of TNF-alpha/IL-23 dual inhibitor (405 mg).

| | | Visit | | | | |
|---|---|---|---|---|---|---|
| Analyte: | | D-1: | D1: | | | D4: |
| Liberated-IL-23 | | Pre | Post | D2 | D3 | 72 Hr |
| Cohort | | Concentration (ng/mL) | | | | |
| Cohort 2 | nObs | 5 | 3 | 7 | 7 | 7 |
| | N(<BLQ) | 5 | 3 | 5 | 5 | 4 |
| | Mean | NR | NR | 16550 | 15100 | 5900 |
| | Min | NR | NR | 1800 | 1510 | 494.5 |
| | Median | NR | NR | 16550 | 15100 | 800 |
| | Max | NR | NR | 31250 | 28650 | 16450 |

NR—No result

TABLE 11
Summary fecal concentrations of liberated-IL-23 following
a single dose of TNF-alpha/IL-23 dual inhibitor (1215 mg).

| | | Visit | | | | | |
|---|---|---|---|---|---|---|---|
| Analyte: | | D-1: | D1: | | | D4: | Unsch |
| Liberated-IL-23 | | Pre | Post | D2 | D3 | 72 Hr | 1 |
| Cohort | | Concentration (ng/mL) | | | | | |
| Cohort 3 | nObs | 1 | 4 | 6 | 7 | 8 | 2 |
| | N(<BLQ) | 1 | 4 | 0 | 2 | 8 | 0 |
| | Mean | NR | NR | 56000 | 3065 | NR | 49700 |
| | Min | NR | NR | 1180 | 740 | NR | 48200 |
| | Median | NR | NR | 38550 | 1905 | NR | 49700 |
| | Max | NR | NR | 129000 | 9650 | NR | 51000 |

NR—No result

TABLE 12
Summary fecal concentrations of liberated-IL-23 following
a single dose of TNF-alpha/IL-23 dual inhibitor (3645 mg).

| | | Visit | | | | | |
|---|---|---|---|---|---|---|---|
| Analyte: | | D-1: | D1: | | | D4: | Unsch |
| Liberated-IL-23 | | Pre | Post | D2 | D3 | 72 Hr | 1 |
| Cohort | | Concentration (ng/mL) | | | | | |
| Cohort | nObs | 3 | 1 | 4 | 4 | 7 | 2 |
| 4 | N(<BLQ) | 3 | 1 | 3 | 2 | 4 | 0 |
| | Mean | NR | NR | 40500 | 10050 | 43050 | 4585 |
| | Min | NR | NR | 40500 | 8950 | 2765 | 640 |
| | Median | NR | NR | 40500 | 10050 | 19700 | 4585 |
| | Max | NR | NR | 40500 | 11150 | 106500 | 8550 |

NR—No result

TABLE 13
Summary fecal concentrations of TNF-alpha/IL-23
dual inhibitor following a single dose of
TNF-alpha/IL-23 dual inhibitor (135 mg).

| | | Visit | | | | | |
|---|---|---|---|---|---|---|---|
| Analyte: TNF-alpha/ | | D-1: | D1: | | | D4: | Unsch |
| IL-23 dual inhibitor | | Pre | Post | D2 | D3 | 72 Hr | 1 |
| Cohort | | Concentration (ng/mL) | | | | | |
| 1 | nObs | 7 | 5 | 5 | 5 | 8 | 1 |
| | N(<BLQ) | 7 | 5 | 5 | 5 | 8 | 1 |
| | Mean | NR | NR | NR | NR | NR | NR |
| | Min | NR | NR | NR | NR | NR | NR |
| | Median | NR | NR | NR | NR | NR | NR |
| | Max | NR | NR | NR | NR | NR | NR |

NR—No result

TABLE 14
Summary fecal concentrations of TNF-alpha/IL-23
dual inhibitor following a single dose of
TNF-alpha/IL-23 dual inhibitor (405 mg).

| | | Visit | | | | | |
|---|---|---|---|---|---|---|---|
| Analyte: TNF-alpha/ | | D-1: | D1: | | | D4: | Unsch |
| IL-23 dual inhibitor | | Pre | Post | D2 | D3 | 72 Hr | 1 |
| Cohort | | Concentration (ng/mL) | | | | | |
| 2 | nObs | 5 | 3 | 7 | 7 | 7 | NR |
| | N(<BLQ) | 5 | 3 | 5 | 5 | 6 | NR |
| | Mean | NR | NR | 20.4 | 19.5 | 15.65 | NR |
| | Min | NR | NR | 17.75 | 14.75 | 15.65 | NR |
| | Median | NR | NR | 20.4 | 19.5 | 15.65 | NR |
| | Max | NR | NR | 23.05 | 24.25 | 15.65 | NR |

NR—No result

TABLE 15
Summary fecal concentrations of TNF-alpha/IL-23
dual inhibitor following a single dose of
TNF-alpha/IL-23 dual inhibitor (1215 mg).

| | | Visit | | | | | |
|---|---|---|---|---|---|---|---|
| Analyte: TNF-alpha/ | | D-1: | D1: | | | D4: | Unsch |
| IL-23 dual inhibitor | | Pre | Post | D2 | D3 | 72 Hr | 1 |
| Cohort | | Concentration (ng/mL) | | | | | |
| 3 | nObs | 1 | 4 | 6 | 7 | 8 | 2 |
| | N(<BLQ) | 1 | 4 | 2 | 4 | 8 | 1 |
| | Mean | NR | NR | 74.5 | 48.95 | NR | 129 |
| | Min | NR | NR | 19.25 | 30.45 | NR | 129 |
| | Median | NR | NR | 46.8 | 45.25 | NR | 129 |
| | Max | NR | NR | 184.5 | 71 | NR | 129 |

NR—No result

TABLE 16
Summary fecal concentrations of TNF-alpha/IL-23
dual inhibitor following a single dose of
TNF-alpha/IL-23 dual inhibitor (3645 mg).

| | | Visit | | | | | |
|---|---|---|---|---|---|---|---|
| Analyte: TNF-alpha/ | | D-1: | D1: | | | D4: | Unsch |
| IL-23 dual inhibitor | | Pre | Post | D2 | D3 | 72 Hr | 1 |
| Cohort | | Concentration (ng/mL) | | | | | |
| 4 | nObs | 2 | 1 | 4 | 4 | 7 | 2 |
| | N(<BLQ) | 3 | 1 | 3 | 1 | 5 | 0 |
| | Mean | NR | NR | 29.55 | 48.2 | 44.15 | 18.05 |
| | Min | NR | NR | 29.55 | 32.8 | 18.4 | 12.85 |
| | Median | NR | NR | 29.55 | 48.2 | 44.15 | 18.05 |
| | Max | NR | NR | 29.55 | 63.5 | 70 | 23.25 |

NR—No result

Part 2 of the clinical study evaluated the safety and tolerability of MD of TNF-alpha/IL-23 dual inhibitor, the concentration of intact TNF-alpha/IL-23 dual inhibitor and its monomers (liberated-TNFliberated-TNF and dual-IL-23) in serum, feces, and urine, and the incidence of anti-drug antibodies. Healthy subjects were randomized 8:2 to active (approximately 1200 mg PO, twice daily) and placebo (PO, twice daily) treatment administered for 7 days. Safety and tolerability, fecal, serum and urine concentrations of TNF-alpha/IL-23 dual inhibitor and its monomers, and ADA levels were assessed at predetermined times. No aEs were observed in Part 2. TNF-alpha/IL-23 dual inhibitor and its monomers were not detected in serum. Fecal concentrations of active TNF-alpha/IL-23 dual inhibitor monomers were observed during all dosing days (Tables 17-18). The highest fecal concentrations observed were liberated-TNFliberated-TNF≈1,700 μg/mL and liberated-IL-23≈5,400 μg/mL (Tables 17-18). Monomer fecal concentrations were observed across all dosing days, peaking at >1700 μg/mL (Tables 17-18).

TABLE 17
Summary fecal concentrations of liberated-TNFliberated-TNF following a twice
daily dose of 1215 mg TNF-alpha/IL-23 dual inhibitor (total 2430 mg).

| | | Visit | | | | | | | | | |
|---|---|---|---|---|---|---|---|---|---|---|---|
| | | Day- | Day | Day | Day | Day | Day | Day | Day | Day | |
| | | 1 | 1 | 2 | 3 | 4 | 5 | 6 | 7 | 8 | Unscheduled |
| Analyte | | Concentration (ng/mL) | | | | | | | | | |
| Liberated- | nObs | 5 | 6 | 6 | 6 | 6 | 7 | 7 | 8 | 1 | 4 |
| IL-23 | N(<BLQ) | 5 | 3 | 3 | 1 | 2 | 1 | 1 | 2 | 0 | 1 |
| | Mean | NR | 221000 | 64500 | 159000 | 29550 | 125500 | 55500 | 207000 | 1705000 | 279000 |
| | Min | NR | 442.5 | 700 | 414 | 540 | 905 | 785 | 970 | 1705000 | 392.5 |
| | Median | NR | 443 | 30800 | 25950 | 1455 | 1670 | 3240 | 54000 | 1705000 | 420.5 |
| | Max | NR | 660000 | 162000 | 487500 | 114500 | 620000 | 168500 | 910000 | 1705000 | 835000 |

NR—No Result

TABLE 18
Summary fecal concentrations of liberated-IL-23 following a twice daily
dose of 1215 mg TNF-alpha/IL-23 dual inhibitor (total 2430 mg).

| | | Visit | | | | | | | | | |
|---|---|---|---|---|---|---|---|---|---|---|---|
| | | Day- | Day | Day | Day | Day | Day | Day | Day | Day | |
| | | 1 | 1 | 2 | 3 | 4 | 5 | 6 | 7 | 8 | Unscheduled |
| Analyte | | Concentration (ng/mL) | | | | | | | | | |
| Liberated- | nObs | 6 | 6 | 6 | 6 | 6 | 7 | 7 | 8 | 1 | 3 |
| IL-23 | N(<BLQ) | 6 | 5 | 3 | 3 | 2 | 1 | 1 | 2 | 0 | 1 |
| | Mean | NR | 358500 | 316000 | 1095000 | 164500 | 1190000 | 366500 | 355500 | 96500 | 110500 |
| | Min | NR | 358500 | 2680 | 3300 | 1510 | 2720 | 2900 | 4560 | 96500 | 1855 |
| | Median | NR | 358500 | 22550 | 1240000 | 3385 | 433500 | 313000 | 217500 | 96500 | 110500 |
| | Max | NR | 358500 | 920000 | 2045000 | 650000 | 5400000 | 800000 | 1180000 | 96500 | 219000 |

NR—No Result

TABLE 19
Summary fecal concentrations of TNF-alpha/IL-23 dual inhibitor following a
twice daily dose of 1215 mg TNF-alpha/IL-23 dual inhibitor (total 2430 mg).

| | | Visit | | | | | | | | | |
|---|---|---|---|---|---|---|---|---|---|---|---|
| | | Day- | Day | Day | Day | Day | Day | Day | Day | Day | |
| | | 1 | 1 | 2 | 3 | 4 | 5 | 6 | 7 | 8 | Unscheduled |
| Analyte | | Concentration (ng/mL) | | | | | | | | | |
| TNF-alpha/ | nObs | 5 | 6 | 6 | 6 | 6 | 7 | 7 | 8 | 1 | 4 |
| IL-23 dual | N(<BLQ) | 5 | 6 | 5 | 4 | 3 | 2 | 2 | 3 | 1 | 4 |
| inhibitor | Mean | NR | NR | 166 | 28.45 | 122.5 | 167 | 132.5 | 82.5 | NR | NR |
| | Min | NR | NR | 166 | 15.05 | 32.2 | 22.95 | 31.35 | 21.25 | NR | NR |
| | Median | NR | NR | 166 | 28.45 | 145 | 226.5 | 150.5 | 81.5 | NR | NR |
| | Max | NR | NR | 166 | 41.85 | 189.5 | 308 | 221 | 184 | NR | NR |

NR—No Result

In both Parts 1 and 2, TNF-alpha/IL-23 dual inhibitor fecal concentrations were on average ≥800-fold lower than TNF-alpha/IL-23 dual inhibitor monomer concentrations (Tables 5-19). TNF-alpha/IL-23 dual inhibitor was well tolerated at all tested doses. Systemic exposure of TNF-alpha/IL-23 dual inhibitor and its monomers was not observed, supporting its gut selectivity. Low levels of intact TNF-alpha/IL-23 dual inhibitor and high levels of TNF-alpha/IL-23 dual inhibitor monomers in feces confirmed that TNF-alpha/IL-23 dual inhibitor is efficiently cleaved after oral dosing.

Taken together, the examples show that TNF-alpha/IL-23 dual inhibitor can be an orally administered for the treatment of IBD. TNF-alpha/IL-23 dual inhibitor can be cleaved by HFS and NHP gut matrices into its monomer arms. Liberated-TNFLiberated-TNF and liberated-IL-23 monomer arms can be stable and retain target binding efficacy after exposure to trypsin, HFS, and NHP gut matrices. TNF-alpha inhibitor and IL-23 inhibitor in combination can decrease protein phosphorylation in UC patient biopsies, which can confirm the benefit of dual targeting. TNF-alpha/IL-23 dual inhibitor and its monomer arms can exhibit rapid serum clearance, which may suggest low risk of systemic immunosuppression in IBD patients.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of treating an autoimmune or inflammatory condition in an individual in need thereof, the method comprising:
a) systemic administration of a first inhibitor to the individual in need thereof, wherein the first inhibitor comprises an inhibitor of interleukin-23 (IL-23), an inhibitor of tumor necrosis factor alpha (TNF-alpha), or combinations thereof; and
b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof;
thereby treating the autoimmune or inflammatory condition in the individual in need thereof.

2. The method of claim 1, wherein the first inhibitor or the second inhibitor are substantially resistant to one or more proteases present in the intestinal tract or to one or more yeast proteases.

3. The method of claim 1 or 2, wherein the systemic administration of the first inhibitor is inhalation, subcutaneous, intravenous, or oral.

4. The method of claim 3, wherein the systemic administration of the first inhibitor is inhalation, subcutaneous, or intravenous.

5. The method of claim 3, wherein the systemic administration of the first inhibitor is subcutaneous or intravenous.

6. The method of claim 3, wherein the systemic administration of the first inhibitor is inhalation or intravenous.

7. The method of claim 3, wherein the systemic administration of the first inhibitor is inhalation or subcutaneous.

8. The method of claim 3, wherein the systemic administration of the first inhibitor is oral.

9. The method of claim 3, wherein the systemic administration of the first inhibitor is by inhalation.

10. The method of claim 3, wherein the systemic administration of the first inhibitor is subcutaneous.

11. The method of claim 3, wherein the systemic administration of the first inhibitor is intravenous.

12. The method of claim 3, wherein the systemic administration of the first inhibitor occurs prior to the local administration of the second inhibitor.

13. The method of claim 12, wherein the systemic administration of the first inhibitor occurs prior to the local administration of the second inhibitor by at least 24 hours.

14. The method of claim 12, wherein the systemic administration of the first inhibitor occurs prior to the local administration of the second inhibitor by at least 2 days.

15. The method of claim 12, wherein the systemic administration of the first inhibitor occurs prior to the local administration of the second inhibitor by at least 3 days.

16. The method of claim 12, wherein the systemic administration of the first inhibitor occurs prior to the local administration of the second inhibitor by at least 7 days.

17. The method of claim 12, wherein the systemic administration of the first inhibitor occurs prior to the local administration of the second inhibitor by at least 14 days.

18. The method of claim 12, wherein the systemic administration of the first inhibitor occurs prior to the local administration of the second inhibitor by at least one month.

19. The method of claim 12, wherein the systemic administration of the first inhibitor occurs prior to the local administration of the second inhibitor by at least two months.

20. The method of claim 12, wherein the systemic administration of the first inhibitor occurs prior to the local administration of the second inhibitor by at least three months.

21. The method of claim 12, wherein the systemic administration of the first inhibitor occurs prior to the local administration of the second inhibitor by at least four months.

22. The method of any one of claims 1 to 21, wherein the local administration of the second inhibitor occurs after a clinical benefit is observed after systemic administration of the first inhibitor.

23. The method of claim 22, wherein the clinical benefit comprises: reduction in the severity of diarrhea, reduction in the frequency of diarrhea, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

24. The method of any one of claims 1 to 23, wherein the local administration of the second inhibitor is oral.

25. The method of any one of claims 1 to 24, wherein the first inhibitor comprises an inhibitor of IL-23.

26. The method of claim 25, wherein the inhibitor of IL-23 comprises a small molecule inhibitor of IL-23.

27. The method of claim 26, wherein the small molecule inhibitor of IL-23 comprises STA-5326 or peptide 2305.

28. The method of claim 25, wherein the inhibitor of IL-23 comprises an antibody that binds IL-23 or an IL-23 binding fragment thereof.

29. The method of claim 28, wherein the antibody that binds IL-23 or an IL-23 binding fragment thereof comprises risankizumab, guselkumab, tildrakizumab, briakinumab, brazikumab, mirikizumab, ustekinumab, or combinations thereof.

30. The method of any one of claims 1 to 29, wherein the first inhibitor comprises an inhibitor of TNF-alpha.

31. The method of claim 30, wherein the inhibitor of TNF-alpha comprises a small molecule inhibitor of TNF-alpha.

32. The method of claim 31, wherein the small molecule inhibitor of TNF-alpha comprises SAR441566, TIM1, TIM1c, SPD-304, MYMD-1, or combinations thereof.

33. The method of claim 30, wherein the inhibitor of TNF-alpha comprises an antibody that binds TNF-alpha or a TNF-alpha binding fragment thereof.

34. The method of claim 33, wherein the antibody that binds TNF-alpha or a TNF-alpha binding fragment thereof comprises infliximab, adalimumab, certolizumab pegol, golimumab, or combinations thereof.

35. The method of claim 30, wherein the inhibitor of TNF-alpha comprises a soluble TNF-alpha receptor.

36. The method of claim 35, wherein the soluble TNF-alpha receptor comprises etanercept.

37. The method of any one of claims 1 to 36, wherein the first inhibitor comprises an inhibitor of IL-23 and an inhibitor of TNF-alpha.

38. The method of any one of claims 1 to 37, wherein the inhibitor of IL-23 and an inhibitor of TNF-alpha are administered separately.

39. The method of any one of claims 1 to 38, wherein the second inhibitor comprises an inhibitor of IL-23.

40. The method of claim 39, wherein the inhibitor of IL-23 comprises an antibody that binds IL-23 or an IL-23 binding fragment thereof.

41. The method of claim 40, wherein the antibody that binds IL-23 or an IL-23 binding fragment thereof comprises a VHH.

42. The method of claim 40 or 41, wherein the VHH comprises:
a) a complementarity determining region 1 (CDR1) comprising an amino acid sequence as set forth in any one of SEQ ID NOs: 1, 4, or 7;
b) a complementarity determining region 2 (CDR2) comprising an amino acid sequence as set forth in any one of SEQ ID NOs: 2, 5, or 8; and
c) a complementarity determining region 3 (CDR3) comprising an amino acid sequence as set forth in any one of SEQ ID NOs: 3, 6, or 9.

43. The method of claim 42, wherein the VHH comprises an amino acid sequence at least about 85%, 90%, 95%, 97%, 98%, or 99% identical to that set forth in any one of SEQ ID NOs: 10-12.

44. The method of any one of claims 1 to 43, wherein the second inhibitor comprises an inhibitor of TNF-alpha.

45. The method of claim 44, wherein the inhibitor of TNF-alpha comprises an antibody that binds TNF-alpha or a TNF-alpha binding fragment thereof.

46. The method of claim 45, wherein the antibody that binds TNF-alpha or an TNF-alpha binding fragment thereof comprises a VHH.

47. The method of claim 46, wherein the VHH comprises:
a) a complementarity determining region 1 (CDR1) comprising an amino acid sequence as set forth in any one of SEQ ID NOs: 101, 107, 108, or 119;
b) a complementarity determining region (CDR2) comprising an amino acid sequence as set forth in any one of SEQ ID NOs: 102, 109-117, or 120; and
c) a complementarity determining region (CDR3) comprising an amino acid sequence as set forth in any one of SEQ ID NOs: 103-106 or 121-124.

48. The method of claim 46, wherein the VHH comprises an amino acid sequence at least about 85%, 90%, 95%, 97%, 98%, or 99% identical to that set forth in any one of SEQ ID NOs: 118 or 125.

49. The method of any one of claims 1 to 48, wherein the second inhibitor comprises a JAK inhibitor.

50. The method of claim 49, wherein the JAK inhibitor comprises a small molecule JAK inhibitor of JAK1, JAK2, JAK3, TYK2, or combinations thereof.

51. The method of claim 49, wherein the JAK inhibitor comprises an antibody or antibody binding fragment thereof that binds to JAK1, JAK2, JAK3, TYK2, or combinations thereof.

52. The method of claim 50, wherein the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

53. The method of any one of claims 1 to 52, wherein the second inhibitor comprises a S1P modulator.

54. The method of claim 53, wherein the S1P modulator is a small molecule S1P modulator.

55. The method of claim 54, wherein the small molecule S1P modulator can agonize or antagonize S1PR1, S1PR2, S1PR3, S1PR4, S1PR5, or combinations thereof.

56. The method of claim 54 or 55, wherein the small molecule S1P modulator comprises fingolimod, ozanimod, siponimod, ponesimod, or combinations thereof.

57. The method of any one of claims 1 to 56, wherein the second inhibitor comprises an inhibitor of TL1A.

58. The method of claim 57, wherein the inhibitor of TL1A comprises a small molecule inhibitor of TL1A.

59. The method of claim 57, wherein the inhibitor of TL1A comprises an antibody or antibody fragment thereof that binds to TL1A.

60. The method of claim 59, wherein the antibody or antibody binding fragment thereof that binds to TL1A comprises PRA023, PF-06480605, or combinations thereof.

61. The method of any one of claims 1 to 60, wherein the first inhibitor and the second inhibitor are administered separately.

62. The method of any one of claims 1 to 60, wherein the first inhibitor and the second inhibitor are administered simultaneously.

63. The method of any one of claims 1 to 62, wherein the first inhibitor and the second inhibitor is an inhibitor of IL-23 and TNF-alpha.

64. The method of claim 63, wherein the inhibitor of IL-23 and the inhibitor of TNF-alpha comprises a polypeptide that binds to both IL-23 and TNF-alpha.

65. The method of claim 64, wherein the polypeptide that binds to both IL-23 and TNF-alpha comprises a first binding region that binds to IL-23 and a second binding region that binds to TNF-alpha, wherein the first binding region that binds to IL-23 comprises:
a) a CDR1 comprising an amino acid sequence as set forth in any one of SEQ ID NOs: 1, 4, or 7;
b) a CDR2 comprising an amino acid sequence as set forth in any one of SEQ ID NOs: 2, 5, or 8; and
c) a CDR3 comprising an amino acid sequence as set forth in any one of SEQ ID NOs: 3, 6, or 9;
wherein the second binding region that binds to TNF-alpha comprises:
a) a CDR1 comprising an amino acid sequence as set forth in any one of SEQ ID NOs: 101, 107, 108, or 119;
b) a CDR2 comprising an amino acid sequence as set forth in any one of SEQ ID NOs: 102, 109-117, or 120; and
c) a CDR3 comprising an amino acid sequence as set forth in any one of SEQ ID NOs: 103-106 or 121-124.

66. The method of claim 65, wherein the first binding region comprises an amino acid sequence at least about 85%, 90%, 95%, 97%, 98%, or 99% identical to that set forth in any one of SEQ ID 10-12; and wherein the second binding region comprises an amino acid sequence at least about 85%, 90%, 95%, 97%, 98%, or 99% identical to that set forth in any one of SEQ ID NOs: 118 or 125.

67. The method of claim 64 or 65, wherein the first binding region and the second binding region are coupled by a protease liable linker.

68. The method of any one of claims 1 to 67, further comprising administration of a third inhibitor wherein the third inhibitor comprises an inhibitor of IL-23, an inhibitor of TNF-alpha, a JAK inhibitor, a modulator of S1P, a TL1A inhibitor, or combinations thereof.

69. The method of claim 68, wherein administration of the third inhibitor is local.

70. The method of claim 68, wherein administration of the third inhibitor is oral.

71. The method of any one of claims 68 to 70, wherein the local administration of the third inhibitor occurs prior to the local administration of the second inhibitor.

72. The method of claim 71, wherein the local administration of the third inhibitor occurs prior to the local administration of the second inhibitor by at least 24 hours.

73. The method of claim 71, wherein the local administration of the third inhibitor occurs prior to the local administration of the second inhibitor by at least 2 days.

74. The method of claim 71, wherein the local administration of the third inhibitor occurs prior to the local administration of the second inhibitor by at least 3 days.

75. The method of claim 71, wherein the local administration of the third inhibitor occurs prior to the local administration of the second inhibitor by at least 7 days.

76. The method of claim 71, wherein the local administration of the third inhibitor occurs prior to the local administration of the second inhibitor by at least 14 days.

77. The method of claim 71, wherein the local administration of the third inhibitor occurs prior to the local administration of the second inhibitor by at least one month.

78. The method of claim 71, wherein the local administration of the third inhibitor occurs prior to the local administration of the second inhibitor by at least two months.

79. The method of claim 71, wherein the local administration of the third inhibitor occurs prior to the local administration of the second inhibitor by at least three months.

80. The method of claim 71, wherein the local administration of the third inhibitor occurs prior to the local administration of the second inhibitor by at least four months.

81. The method of any one of claims 68 to 80, wherein the third inhibitor comprises an inhibitor of IL-23.

82. The method of claim 81, wherein the inhibitor of IL-23 comprises an antibody that binds IL-23 or an IL-23 binding fragment thereof.

83. The method of claim 82, wherein the antibody that binds IL-23 or an IL-23 binding fragment thereof comprises a VHH.

84. The method of claim 81 or 82, wherein the VHH comprises:
a) a CDR1 comprising an amino acid sequence as set forth in any one of SEQ ID NOs: 1, 4, or 7;
b) a CDR2 comprising an amino acid sequence as set forth in any one of SEQ ID NOs: 2, 5, or 8; and
c) a CDR3 comprising an amino acid sequence as set forth in any one of SEQ ID NOs: 3, 6, or 9.

85. The method of claim 84, wherein the VHH comprises an amino acid sequence at least about 85%, 90%, 95%, 97%, 98%, or 99% identical to that set forth in any one of SEQ ID NOs: 10-12.

86. The method of any one of claims 68 to 85, wherein the third inhibitor comprises an inhibitor of TNF-alpha.

87. The method of claim 86, wherein the inhibitor of TNF-alpha comprises an antibody that binds TNF-alpha or a TNF-alpha binding fragment thereof.

88. The method of claim 87, wherein the antibody that binds TNF-alpha or an TNF-alpha binding fragment thereof comprises a VHH.

89. The method of claim 88, wherein the VHH comprises:
a) a CDR1 comprising an amino acid sequence as set forth in any one of SEQ ID NOs: 101, 107, 108, or 119;
b) a CDR2 comprising an amino acid sequence as set forth in any one of SEQ ID NOs: 102, 109-117, or 120; and
c) a CDR3 comprising an amino acid sequence as set forth in any one of SEQ ID NOs: 103-106 or 121-124.

90. The method of claim 88, wherein the VHH comprises an amino acid sequence at least about 85%, 90%, 95%, 97%, 98%, or 99% identical to that set forth in any one of SEQ ID NOs: 118 or 125.

91. The method of any one of claims 68 to 90, wherein the third inhibitor is an inhibitor of IL-23 and TNF-alpha.

92. The method of claim 91, wherein the inhibitor of IL-23 and the inhibitor of TNF-alpha comprises a polypeptide that binds to both IL-23 and TNF-alpha.

93. The method of claim 92, wherein the polypeptide that binds to both IL-23 and TNF-alpha comprises a first binding region that binds to IL-23 and a second binding region that binds to TNF-alpha, wherein the first binding region that binds to IL-23 comprises:
a) a CDR1 comprising an amino acid sequence as set forth in any one of SEQ ID NOs: 1, 4, or 7;
b) a CDR2 comprising an amino acid sequence as set forth in any one of SEQ ID NOs: 2, 5, or 8; and
c) a CDR3 comprising an amino acid sequence as set forth in any one of SEQ ID NOs: 3, 6, or 9;
wherein the second binding region that binds to TNF-alpha comprises:
a) a CDR1 comprising an amino acid sequence as set forth in any one of SEQ ID NOs: 101, 107, 108, or 119;
b) a CDR2 comprising an amino acid sequence as set forth in any one of SEQ ID NOs: 102, 109-117, or 120; and
c) a CDR3 comprising an amino acid sequence as set forth in any one of SEQ ID NOs: 103-106 or 121-124.

94. The method of claim 93, wherein the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125.

95. The method of claim 93 or 94, wherein the first binding region and the second binding region are coupled by a protease liable linker.

96. The method of any one of claims 68 to 95, wherein the third inhibitor comprises a JAK inhibitor.

97. The method of claim 96, wherein the JAK inhibitor comprises a small molecule JAK inhibitor of JAK1, JAK2, JAK3, TYK2, or combinations thereof.

98. The method of claim 97, wherein the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

99. The method of claim 96, wherein the JAK inhibitor comprises an antibody or antibody fragment that binds to JAK1, JAK2, JAK3, TYK2, or combinations thereof.

100. The method of any one of claims 68 to 99, wherein the third inhibitor comprises a S1P modulator.

101. The method of claim 100, wherein the S1P modulator is a small molecule S1P modulator.

102. The method of claim 101, wherein the small molecule S1P modulator can agonize or antagonize S1PR1, S1PR2, S1PR3, S1PR4, S1PR5, or combinations thereof.

103. The method of claim 100 or 101, wherein the small molecule S1P modulator comprises fingolimod, ozanimod, siponimod, ponesimod, or combinations thereof.

104. The method of any one of claims 68 to 103, wherein the third inhibitor comprises an inhibitor of TL1A.

105. The method of claim 104, wherein the inhibitor of TL1A comprises a small molecule inhibitor of TL1A.

106. The method of claim 104, wherein the inhibitor of TL1A comprises an antibody or antibody fragment thereof that binds to TL1A.

107. The method of claim 106, wherein the antibody or antibody binding fragment thereof that binds to TL1A comprises PRA023, PF-06480605, or combinations thereof.

108. The method of any one of claims 1 to 107, wherein the autoimmune or inflammatory condition is an autoimmune or inflammatory condition of the gastrointestinal tract.

109. The method of claim 108, wherein the autoimmune or inflammatory condition of the gastrointestinal tract comprise inflammatory bowel disease or Crohn's disease.

110. A method of treating an autoimmune or inflammatory condition in an individual in need thereof, the method comprising:
a) systemic administration of a first inhibitor to the individual in need thereof, wherein the first inhibitor comprises an inhibitor of interleukin-23 (IL-23) and/or an inhibitor of tumor necrosis factor alpha (TNF-alpha); and
b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof;
thereby treating the autoimmune or inflammatory condition in the individual in need thereof.

111. The method of claim 110, wherein the systemic administration of the first inhibitor is subcutaneous or intravenous.

112. The method of claim 110, wherein the systemic administration of the first inhibitor is subcutaneous.

113. The method of claim 110, wherein the systemic administration of the first inhibitor is intravenous.

114. The method of any one of claims 110 to 113, wherein the systemic administration of the first inhibitor occurs prior to the local administration of the second inhibitor.

115. The method of claim 114, wherein the systemic administration of the first inhibitor occurs prior to the local administration of the second inhibitor by at least 24 hours.

116. The method of claim 114, wherein the systemic administration of the first inhibitor occurs prior to the local administration of the second inhibitor by at least 2 days.

117. The method of claim 114, wherein the systemic administration of the first inhibitor occurs prior to the local administration of the second inhibitor by at least 3 days.

118. The method of claim 114, wherein the systemic administration of the first inhibitor occurs prior to the local administration of the second inhibitor by at least 7 days.

119. The method of claim 114, wherein the systemic administration of the first inhibitor occurs prior to the local administration of the second inhibitor by at least 14 days.

120. The method of claim 114, wherein the systemic administration of the first inhibitor occurs prior to the local administration of the second inhibitor by at least one month.

121. The method of claim 114, wherein the systemic administration of the first inhibitor occurs prior to the local administration of the second inhibitor by at least two months.

122. The method of claim 114, wherein the systemic administration of the first inhibitor occurs prior to the local administration of the second inhibitor by at least three months.

123. The method of claim 114, wherein the systemic administration of the first inhibitor occurs prior to the local administration of the second inhibitor by at least 4 months.

124. The method of any one of claims 110 to 123, wherein the local administration of the second inhibitor is oral.

125. The method of any one of claims 110 to 123, wherein the first inhibitor comprises an inhibitor of IL-23.

126. The method of claim 125, wherein the inhibitor of IL-23 comprises a small molecule inhibitor of IL-23.

127. The method of claim 126, wherein the small molecule inhibitor of IL-23 comprises STA-5326 or peptide 2305.

128. The method of claim 125, wherein the inhibitor of IL-23 comprises an antibody that binds IL-23 or an IL-23 binding fragment thereof.

129. The method of claim 128, wherein the antibody that binds IL-23 or an IL-23 binding fragment thereof comprises risankizumab, guselkumab, tildrakizumab, briakinumab, brazikumab, mirikizumab, ustekinumab, or combinations thereof.

130. The method of any one of claims 110 to 129, wherein the first inhibitor comprises an inhibitor of TNF-alpha.

131. The method of claim 130, wherein the inhibitor of TNF-alpha comprises a small molecule inhibitor of TNF-alpha.

132. The method of claim 131, wherein the small molecule inhibitor of TNF-alpha comprises SAR441566, TIM1, TIM1c, SPD-304, MYMD-1, or combinations thereof.

133. The method of claim 130, wherein the inhibitor of TNF-alpha comprises an antibody that binds TNF-alpha or a TNF-alpha binding fragment thereof.

134. The method of claim 133, wherein the antibody that binds TNF-alpha or a TNF-alpha binding fragment thereof comprises infliximab, adalimumab, certolizumab pegol, golimumab, or combinations thereof.

135. The method of claim 130, wherein the inhibitor of TNF-alpha comprises a soluble TNF-alpha receptor.

136. The method of claim 135, wherein the soluble TNF-alpha receptor comprises etanercept.

137. The method of any one of claims 110 to 136, wherein the inhibitor of IL-23 and the inhibitor of TNF-alpha comprises a dual inhibitor of interleukin-23 (IL-23) and tumor necrosis factor alpha (TNF-alpha) comprises a first binding region that binds to IL-23 and a second binding region that binds to TNF-alpha, wherein the first binding region that binds to IL-23 comprises:
a) a CDR1 comprising an amino acid sequence as set forth in any one of SEQ ID NOs: 1, 4, or 7;
b) a CDR2 comprising an amino acid sequence as set forth in any one of SEQ ID NOs: 2, 5, or 8; and
c) a CDR3 comprising an amino acid sequence as set forth in any one of SEQ ID NOs: 3, 6, or 9;
wherein the second binding region that binds to TNF-alpha comprises:
a) a CDR1 comprising an amino acid sequence as set forth in any one of SEQ ID NOs: 101, 107, 108, or 119;
b) a CDR2 comprising an amino acid sequence as set forth in any one of SEQ ID NOs: 102, 109-117, or 120; and
c) a CDR3 comprising an amino acid sequence as set forth in any one of SEQ ID NOs: 103-106 or 121-124.

138. The method of claim 137, wherein the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125.

139. The method of any one of claims 110 to 138, wherein the second inhibitor comprises a JAK inhibitor.

140. The method of claim 139, wherein the JAK inhibitor comprises a small molecule JAK inhibitor of JAK1, JAK2, JAK3, TYK2, or combinations thereof.

141. The method of claim 139, wherein the JAK inhibitor comprises an antibody or antibody binding fragment thereof that binds to JAK1, JAK2, JAK3, TYK2, or combinations thereof.

142. The method of claim 140, wherein the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

143. The method of any one of claims 110 to 142, wherein the second inhibitor comprises a S1P modulator.

144. The method of claim 143, wherein the S1P modulator is a small molecule S1P modulator.

145. The method of claim 144, wherein the small molecule S1P modulator can agonize or antagonize S1PR1, S1PR2, S1PR3, S1PR4, S1PR5, or combinations thereof.

146. The method of claim 143 or 144, wherein the small molecule S1P modulator comprises fingolimod, ozanimod, siponimod, ponesimod, or combinations thereof.

147. The method of any one of claims 110 to 146, wherein the second inhibitor comprises an inhibitor of TL1A.

148. The method of claim 147, wherein the inhibitor of TL1A comprises a small molecule inhibitor of TL1A.

149. The method of claim 147, wherein the inhibitor of TL1A comprises an antibody or antibody fragment thereof that binds to TL1A.

150. The method of claim 149, wherein the antibody or antibody binding fragment thereof that binds to TL1A comprises PRA023, PF-06480605, or combinations thereof.

151. The method of claim 62, wherein systemic administration of the first inhibitor is ended after 1 week, 2 weeks, 3 weeks, 4 weeks, 2 months, 3 months, or 4 months after the start of the systemic administration.

152. The method of claim 62, wherein systemic administration of the first inhibitor is ended after a clinical benefit is observed after systemic administration of the first inhibitor.

153. The method of claim 152, wherein the clinical benefit comprises: reduction in the severity of diarrhea, reduction in the frequency of diarrhea, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

154. The method of any one of claims 1 to 60, wherein the first inhibitor and the second inhibitor are administered sequentially.

155. The method of any one of claims 110 to 114, wherein the local administration of the second inhibitor occurs after a clinical benefit is observed after systemic administration of the first inhibitor.

156. A method of treating an autoimmune and/or inflammatory condition in an individual in need thereof, the method comprising:
a) systemic administration of a first inhibitor to the individual in need thereof, wherein the first inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof; and
b) local administration of a second inhibitor to the individual in need thereof, wherein the second inhibitor comprises an inhibitor of interleukin-23 (IL-23), an inhibitor of tumor necrosis factor alpha (TNF-alpha), a Janus kinase (JAK) inhibitor, a sphingosine-1 phosphate (S1P) modulator, an inhibitor of TNF-like ligand 1A (TL1A), or combinations thereof;
thereby treating the autoimmune and/or inflammatory condition in the individual in need thereof.

157. The method of claim 156, wherein the systemic administration of the first inhibitor is subcutaneous or intravenous.

158. The method of claim 156, wherein the systemic administration of the first inhibitor is subcutaneous.

159. The method of claim 156, wherein the systemic administration of the first inhibitor is intravenous.

160. The method of any one of claims 156 to 159, wherein the systemic administration of the first inhibitor occurs prior to the local administration of the second inhibitor.

161. The method of claim 160, wherein the systemic administration of the first inhibitor occurs prior to the local administration of the second inhibitor by at least 24 hours.

162. The method of claim 160, wherein the systemic administration of the first inhibitor occurs prior to the local administration of the second inhibitor by at least 2 days.

163. The method of claim 160, wherein the systemic administration of the first inhibitor occurs prior to the local administration of the second inhibitor by at least 3 days.

164. The method of claim 160, wherein the systemic administration of the first inhibitor occurs prior to the local administration of the second inhibitor by at least 7 days.

165. The method of claim 160, wherein the systemic administration of the first inhibitor occurs prior to the local administration of the second inhibitor by at least 14 days.

166. The method of claim 160, wherein the systemic administration of the first inhibitor occurs prior to the local administration of the second inhibitor by at least one month.

167. The method of claim 160, wherein the systemic administration of the first inhibitor occurs prior to the local administration of the second inhibitor by at least two months.

168. The method of claim 160, wherein the systemic administration of the first inhibitor occurs prior to the local administration of the second inhibitor by at least three months.

169. The method of claim 160, wherein the systemic administration of the first inhibitor occurs prior to the local administration of the second inhibitor by at least 4 months.

170. The method of any one of claims 156 to 169, wherein the local administration of the second inhibitor is oral.

171. The method of any one of claims 156 to 170, wherein the first inhibitor comprises an inhibitor of IL-23.

172. The method of claim 171, wherein the inhibitor of IL-23 comprises a small molecule inhibitor of IL-23.

173. The method of claim 172, wherein the small molecule inhibitor of IL-23 comprises STA-5326, peptide 2305, or combinations thereof.

174. The method of claim 171, wherein the inhibitor of IL-23 comprises an antibody that binds IL-23 or an IL-23 binding fragment thereof.

175. The method of claim 174, wherein the antibody that binds IL-23 or an IL-23 binding fragment thereof comprises risankizumab, guselkumab, tildrakizumab, briakinumab, brazikumab, mirikizumab, ustekinumab, or combinations thereof.

176. The method of any one of claims 156 to 175, wherein the first inhibitor comprises an inhibitor of TNF-alpha.

177. The method of claim 176, wherein the inhibitor of TNF-alpha comprises a small molecule inhibitor of TNF-alpha.

178. The method of claim 177, wherein the small molecule inhibitor of TNF-alpha comprises SAR441566, TIM1, TIM1c, SPD-304, MYMD-1, or combinations thereof.

179. The method of claim 176, wherein the inhibitor of TNF-alpha comprises an antibody that binds TNF-alpha or a TNF-alpha binding fragment thereof.

180. The method of claim 179, wherein the antibody that binds TNF-alpha or a TNF-alpha binding fragment thereof comprises infliximab, adalimumab, certolizumab pegol, golimumab, or combinations thereof.

181. The method of claim 176, wherein the inhibitor of TNF-alpha comprises a soluble TNF-alpha receptor.

182. The method of claim 181, wherein the soluble TNF-alpha receptor comprises etanercept.

183. The method of any one of claims 156 to 182, wherein the inhibitor of IL-23 and the inhibitor of TNF-alpha comprises a dual inhibitor of interleukin-23 (IL-23) and tumor necrosis factor alpha (TNF-alpha) comprises a first binding region that binds to IL-23 and a second binding region that binds to TNF-alpha, wherein the first binding region that binds to IL-23 comprises:
a) a CDR1 comprising an amino acid sequence as set forth in any one of SEQ ID NOs: 1, 4, or 7;
b) a CDR2 comprising an amino acid sequence as set forth in any one of SEQ ID NOs: 2, 5, or 8; and
c) a CDR3 comprising an amino acid sequence as set forth in any one of SEQ ID NOs: 3, 6, or 9;
wherein the second binding region that binds to TNF-alpha comprises:
a) a CDR1 comprising an amino acid sequence as set forth in any one of SEQ ID NOs: 101, 107, 108, or 119;
b) a CDR2 comprising an amino acid sequence as set forth in any one of SEQ ID NOs: 102, 109-117, or 120; and
c) a CDR3 comprising an amino acid sequence as set forth in any one of SEQ ID NOs: 103-106 or 121-124.

184. The method of claim 183, wherein the first binding region comprises an amino acid sequence at least about 85%, 90%, 95%, 97%, 98%, or 99% identical to that set forth in any one of SEQ ID NOs: 10-12; and wherein the second binding region comprises an amino acid sequence at least about 85%, 90%, 95%, 97%, 98%, or 99% identical to that set forth in any one of SEQ ID NOs: 118 or 125.

185. The method of any one of claims 156 to 184, wherein the second inhibitor comprises a JAK inhibitor.

186. The method of claim 185, wherein the JAK inhibitor comprises a small molecule JAK inhibitor of JAK1, JAK2, JAK3, TYK2, or combinations thereof.

187. The method of claim 185, wherein the JAK inhibitor comprises an antibody or antibody binding fragment thereof that binds to JAK1, JAK2, JAK3, TYK2, or combinations thereof.

188. The method of claim 186, wherein the small molecule JAK inhibitor comprises abrocitinib, baricitinib, upadacitinib ruxolitinib, tofacitinib, oclacitinib, peficitinib, fedratinib, filgotinib, pacritinib, deucravacitinib, ritlecitinib, cerdulatinib, gandotinib, lestaurtinib, momelotinib, TAK-279, VTX958, ESK-001, or combinations thereof.

189. The method of any one of claims 156 to 188, wherein the second inhibitor comprises a S1P modulator.

190. The method of claim 189, wherein the S1P modulator is a small molecule S1P modulator.

191. The method of claim 190, wherein the small molecule S1P modulator can agonize or antagonize S1PR1, S1PR2, S1PR3, S1PR4, S1PR5, or combinations thereof.

192. The method of claim 189 or 190, wherein the small molecule S1P modulator comprises fingolimod, ozanimod, siponimod, ponesimod, etrasimod, tamuzimod, amiselimod or combinations thereof.

193. The method of any one of claims 156 to 192, wherein the second inhibitor comprises an inhibitor of TL1A.

194. The method of claim 193, wherein the inhibitor of TL1A comprises a small molecule inhibitor of TL1A.

195. The method of claim 193, wherein the inhibitor of TL1A comprises an antibody or antibody fragment thereof that binds to TL1A.

196. The method of claim 195, wherein the antibody or antibody binding fragment thereof that binds to TL1A comprises PRA023, PF-06480605, TEV-48574, or combinations thereof.

197. The method of any one of claims 156 to 160, wherein the local administration of the second inhibitor occurs after a clinical benefit is observed after systemic administration of the first inhibitor.

198. The method of claim 197, wherein the clinical benefit comprises: reduction in the severity of diarrhea, reduction in the frequency of diarrhea, reduction in the frequency of urgency, improvement in patient stool consistency, reduction of perianal conditions, reduction in the severity of rectal bleeding, reduction in the frequency of rectal bleeding, reduction in the amount of blood and/or mucus in patient stool, reduction in the severity of abdominal cramping, reduction in the frequency of abdominal cramping, reduction in the frequency of abdominal pain, improvement in hematological parameters, improvement in fecal calprotectin concentration, improvement in serological parameter, improvement in gross features of the gastrointestinal (GI) tract, reduction in the severity of fatigue, reduction in the frequency of fatigue, reduction in the severity of fevers, reduction in the frequency of fevers, prevention of unintended weight loss, prevention of pain, an improvement in appetite, improvement in sleep quality, improvement in quality of life, or combinations thereof.

199. The method of any one of claims 156 to 184, wherein the second inhibitor comprises a TYK2 inhibitor.

200. The method of claim 199, wherein the TYK2 inhibitor comprises comprises TAK-279, VTX958, ESK-001, or combinations thereof.

201. The method of claim 160, wherein the systemic administration of the first inhibitor occurs prior to the local administration of the second inhibitor by at least 12 months.

202. The method of claim 172, wherein the small molecule inhibitor of IL-23 comprises a peptide.

203. The method of claim 171, wherein the inhibitor of IL-23 comprises a miniprotein.

204. The method of claim 203, wherein the miniprotein comprises an IL-23R minibinder.

205. The method of claim 203, wherein the miniprotein comprises JNJ-2113.

206. The method of any one of claims 156 to 203, wherein the autoimmune or inflammatory condition is an autoimmune or inflammatory condition of the gastrointestinal tract.

207. The method of claim 204, wherein the autoimmune or inflammatory condition of the gastrointestinal tract comprise inflammatory bowel disease, Crohn's disease, or ulcerative colitis.
